(19) United States
(12) Patent Application Publication
(10) Pub. No.: US 2019/0367981 A1
(43) Pub. Date: Dec. 5, 2019

(54) PROBE INCLUDING FALSE-POSITIVE-SUPPRESSING FUNCTION, METHOD FOR DESIGNING THE SAME, AND METHOD FOR UTILIZING THE SAME

(71) Applicant: Yokowo Co., Ltd., Kita-ku, Tokyo (JP)

(72) Inventors: Yui Niijima, Kita-ku, Tokyo (JP); Shintaro Takase, Kita-ku, Tokyo (JP); Hisashi Suzuki, Kita-ku, Tokyo (JP); Hisafumi Ikeda, Itabashi-ku, Tokyo (JP)

(73) Assignee: Yokowo Co., Ltd., Kita-ku, Tokyo (JP)

(21) Appl. No.: 16/487,713

(22) PCT Filed: Feb. 20, 2018
(86) PCT No.: PCT/JP2018/005964

§ 371 Date: Aug. 21, 2019

(30) Foreign Application Priority Data
Feb. 22, 2017 (JP) 2017-030553

(51) Int. Cl. C12Q 1/6876

(52) U.S. Cl. CPC C12Q 1/6876; C12Q 2600/178

(57) ABSTRACT

An object of the present invention is to provide a means for detecting or quantitatively determining short-chain nucleic acids by simple double-strand formation with high specificity.

The present invention relates to a polynucleobase probe including, in a sequence complementary to a target sequence having at least one sequence of any one of SEQ ID NOs: 1 to 10, a sequence in which at least one of bases in a portion complementary to any one sequence of SEQ ID NOs: 1 to 10 in the target sequence becomes abasic and/or is substituted; a method for designing the same; and a method for utilizing the same.

### TECHNICAL FIELD

The present invention relates to a probe that can be utilized for nucleic acid detection with high specificity, a method for designing the same, and a method for detecting nucleic acid by utilizing the probe.

### BACKGROUND ART

In recent years, small non-coding RNAs (ncRNAs) containing about 20 bases of microRNAs (miRNAs) have attracted attention because of their various functions. In particular, when these ncRNA levels correlate with diseases, ncRNAs can be utilized as markers.

When binding a target DNA or RNA to a probe by hybridization, a sequence of a binding part of a target DNA or RNA (hereinafter referred to as a “binding sequence”) in which a large number of any one of guanine or cytosine having strong binding power is continuous, may be present in some case. In such a case, a phenomenon occurs in which false positives are likely to be generated due to a probe binding also to non-target DNAs or RNAs which have the same contiguous sequence as this sequence. In the related art, when a probe is bound to a long-chain target DNA or RNA which has such sequences that easily induce a false positive, a probe for a site that does not contain any sequence in which a large number of any one of guanine or cytosine is continuous was designed to be used. In general, it is perceived that a probe length of about 18-mer is necessary for detection of specific sequences by hybridization. However, in a case of detecting a target sequence composed of a short chain length of about 20-mer, such as an miRNA, it is not possible to design a probe that does not contain any sequence in which a large number of any one of guanine or cytosine is continuous, and therefore occurrence of false positives could not be avoided.

For this reason, as a method for detecting an ncRNA containing an miRNA, a method not based on detection of a specific sequence by hybridization, for example, a method utilizing polymerase chain reaction (PCR), oligonucleotide ligation assay (OLA), or ligase chain reaction (LCR) (Patent Document 1), and the like have been used.

In addition, as a method for improving specificity of miRNA measurement, a method using LNA with high affinity (Patent Document 2) is known. However, although this method increases binding power in hybridization, sequences in which a large number of any one of guanine or cytosine is continuous are generated, and therefore false positives could not be avoided.

Patent Document 3 discloses a method of irradiating a double-stranded oligonucleotide with light and detecting a SNP by using a difference in light absorption with use of a probe bound to a light-responsive organic group. The document discloses that any of a sequence in which an SNP site is not mutated and a sequence in which an SNP site is mutated can be used as a probe in the present document. In addition, Patent Document 3 discloses a probe in which a SNP site is substituted with a mutant as a probe for SNP. However, because the mutation is inserted in the SNP site in a target sequence itself to be bound, the sequences of these probes are designed as sequences complementary to the target sequence.

Peptide nucleic acid (PNA) is a DNA/RNA mimetic having a pseudopeptide skeleton in which N-(2-aminoethyl) glycine is bound by an amide bond instead of a sugar chain in DNA or RNA. PNA can be used as a new probe because it can form a double strand with DNA/RNA (Non-patent Document 1 and Non-patent Document 2).

Patent Document 4 discloses a method including a step of forming a double strand with PNA in which a part of bases is deleted and with nucleic acids; a step of reversibly binding by contacting a tagged base to a base-deleted portion (a portion where no pair is formed with a nucleic acid) of double-stranded PNA; and a step of detecting a target nucleic acid by detecting the tagged base. In this method, the label is attached to the tagged base rather than the PNA, and the target nucleic acid is detected by the binding of the tagged base to the PNA.

Non-Patent Document 3 discloses that a 15-mer DNA probe in which single base substitution, abasic site creation, or phenyl-substitution is performed has a lower Tm value, and thus lowers stability of the double strand as compared to a fully complementary probe. In particular, it has been reported that, in a case of a DNA and DNA double helix in which an abasic or phenyl-substituted DNA probe is used, a Tm value is reduced by about 40%, and therefore stability of the double helix is decreased. Non-Patent Document 4 discloses that, when a PNA and DNA double helix is formed, and a Tm value is measured by using an abasic or phenyl-substituted 15-mer PNA probe as above, the Tm value was 4° C. for the case of abasic site creation, and the Tm value was 6.5° C. for the case of phenyl substitution, which are a decrease in the Tm values, and therefore stability is decreased similarly to the DNA probe. In Non-patent document 5, one base of a 19-mer PNA probe is substituted with anthraquinone (AQ) having absorbance at 330 nm for the purpose of modifying the inside of a double helix structure. AQ has been shown to fit within the double strand while maintaining relatively high Tm values, whereas an abasic site has been shown to lower stability of double-strand formation.

### RELATED DOCUMENT

### Patent Document

[Patent Document 1] International Publication No. WO2005/098029

[Patent Document 2] International Publication No. WO2006/069584

[Patent Document 3] Japanese Unexamined Patent Publication No. 2001-346579

[Patent Document 4] International Publication No. WO2009/037473

### Non-patent Document

[Non-patent document 1] Nielsen, P. E. et al., Science; 254: 1497-1500 (1991)

[Non-Patent Document 2] Michael Egholm, et al., Nature; 365: 566-568 (1993)

[Non-Patent Document 3] Millican, et al., Nucleic Acids Research; 12: 7435-7453 (1984)

[Non-patent document 4] Hemavathi Challa, et al., Tetrahedron Letters; 40: 8333-8336 (1999)

[Non-patent document 5] Bruce Armitage, et al., Nucleic Acids Research; 26: 715-720 (1998)

### SUMMARY OF THE INVENTION

### Technical Problem

An object of the present invention is to provide a means capable of detecting a target nucleic acid and a means capable of quantitatively determining a target nucleic acid with a low false positive rate (high specificity) through double-strand formation by hybridization of a probe and a target nucleic acid in detection of a short-chain target nucleic acid having a sequence in which any one of guanine or cytosine is continuous, without requiring complicated procedures such as ligation and amplification which have been employed in the related art. More specifically, an object of the present invention is to reduce a false positive rate in complementary strand formation by hybridization between a probe and a target nucleic acid in which one of guanine and cytosine has a contiguous sequence, so as to improve specificity. In particular, an object of the present invention is to provide a probe which is capable of reducing non-specific binding to a non-target nucleic acid in double-strand formation with a nucleic acid having a 10- to 50-mer target sequence having a sequence in which any one of guanine or cytosine is continuous, and thereby detecting or quantitatively determining a target nucleic acid with high specificity, which was difficult to perform with high specificity. As one example, the target nucleic acid is a miRNA having a sequence in which guanine or cytosine is continuous.

In general, making a sequence that is not complementary to a part of a target sequence by substituting a base of a probe or creating an abasic site reduces binding power to a target nucleic acid. It has been exceptionally reported that binding power is relatively maintained in an example in which one base of a PNA probe is substituted with a specific anthraquinone (AQ) such as 3,6-diaza (N³-Boc-aminoethyl) -4,7-dioxo-7-(2-anthraquinoyl)-hepta noic acid for labeling. However, a sequence that is not complementary to a part of a target sequence has not been adopted in the field of general nucleic acid detection and nucleic acid analysis, the sequence being obtained by substituting a base, creating an abasic site, or cleaving a sequence in a probe for detecting a nucleic acid by double-strand formation. In addition, there has been no concept of suppressing binding to a non-target sequence by substituting a base, creating an abasic site, or cleaving a sequence in a completely complementary probe. Furthermore, PNA has been known to stably form a double strand while being more sensitive to mismatches compared to DNA (described by Michael et al.).

### Solution to Problem

The inventors of the present invention have attempted to design a probe via various approaches without being bound by such a common-sense idea in the field of genetic engineering. As a result, the inventors of the present invention have found that, when bases having strong binding power are continuous even in a case of a short-chain probe, binding power to non-target nucleic acids can be dramatically reduced while maintaining binding power to target nucleic acids by cleaving or substituting a part thereof or creating an abasic site. In addition, the inventors of the present invention have found that such a probe particularly has different binding power between a non-target nucleic acid and a target nucleic acid to the extent that false positives and positives can be distinguished in the detection of target nucleic acids in which bases (guanine and cytosine) with strong binding power are continuous. As a result, obtaining a probe useful for detecting target nucleic acids in which bases (guanine and cytosine) with strong binding power are continuous, and designing the same have been achieved. The present invention has been made based on such findings, and specifically relates to the following inventions.

(1) A polynucleobase probe including, in a sequence complementary to a target sequence having at least one sequence of any one of SEQ ID NOs: 1 to 10, a sequence in which at least one of bases in a portion complementary to any one sequence of SEQ ID NOs: 1 to 10 in the target sequence becomes abasic and/or is substituted; and/or a sequence which is cleaved to have, on an end, at least one sequence complementary to a sequence of 2 bases or less in any one sequence of SEQ ID NOs: 1 to 10 in the target sequence.

(2) The polynucleobase probe according to (1), which is 10- to 50-mer.

(3) The polynucleobase probe according to (2), which is 15- to 28-mer.

(4) The polynucleobase probe according to anyone of (1) to (3), to which a label is bound.

(5) The polynucleobase probe according to anyone of (1) to (4), in which, in the portion complementary to any one sequence of SEQ ID NOs: 1 to 10 in the target sequence, at least one of the bases which become abasic or are substituted is located inside the portion complementary to anyone sequence of SEQ ID NOs: 1 to 10 in the target sequence.

(6) The polynucleobase probe according to anyone of (1) to (5), in which, in the sequence complementary to any one sequence of SEQ ID NOs: 1 to 10 in the target sequence, a ratio of at least one of the bases which become abasic or are substituted with respect to any one of 3 to 5 guanines and cytosines is 1.

(7) The polynucleobase probe according to anyone of (1) to (6), in which the target nucleic acid is a 10- to 50-mer DNA or RNA.

(8) The polynucleobase probe according to (7), in which the target nucleic acid is a miRNA.

(9) The polynucleobase probe according to anyone of (1) to (8), which is a DNA, RNA, LNA, GNA, BNA, or PNA.

(10) A method for designing a polynucleobase probe sequence which is capable of binding to a target sequence having at least one sequence of any one of SEQ ID NOs: 1 to 10 with high specificity, the method including:

A) selecting a 10- to 50-mer sequence fully complementary to the target sequence as a fully complementary probe sequence; and

B) (i) in the fully complementary probe sequence, designing the polynucleobase probe sequence by substituting and/or making at least one of bases abasic in a portion complementary to any one sequence of SEQ ID NOs: 1 to 10 in the target sequence, and/or

(ii) in the fully complementary probe sequence, designing the polynucleobase probe sequence by cleaving an end of the fully complementary probe sequence such that a portion complementary to anyone sequence of SEQ ID NOs: 1 to 10 in the target sequence becomes 2 bases or less.

(11) The method according to (10), in which the polynucleobase probe is 15- to 28-mer.

(12) The method according to (10) or (11), in which design of the polynucleobase probe sequence is performed by creating an abasic site, substituting, or cleaving such that a polynucleobase complementary to anyone sequence of SEQ ID NOs: 1 to 10 in the target sequence becomes 2 bases or less.

(13) A method for detecting a target nucleic acid having at least one sequence of any one of SEQ ID NOs: 1 to 10 in a test sample with high specificity, the method including:

preparing the test sample to detect the target nucleic acid;

bringing at least one kind of the polynucleobase probes according to any one of (1) to (9) in contact with the test sample; and

detecting the target nucleic acid bound to the polynucleobase probe.

(14) A method for quantitatively determining a target nucleic acid having at least one sequence of any one of SEQ ID NOs: 1 to 10 in a test sample with high specificity, the method including:

preparing the test sample to quantitatively determine the target nucleic acid;

contacting at least one kind of the polynucleobase probes according to any one of (1) to (9) with the test sample; and

quantitatively determining the target nucleic acid bound to the polynucleobase probe.

In the present specification, a “sequence in which any one of guanine or cytosine is continuous for 3 or more bases” or a “GC contiguous sequence” means the same as each other, and means GGGGGGG (SEQ ID NO: 1), CCCCCCC (SEQ ID NO: 2), GGGGGG (SEQ ID NO: 3), CCCCCC (SEQ ID NO: 4), GGGGG (SEQ ID NO: 5), CCCCC (SEQ ID NO: 6), GGGG (SEQ ID NO: 7), CCCC (SEQ ID NO: 8), GGG (SEQ ID NO: 9), and CCC (SEQ ID NO: 10). However, the term “GC contiguous sequence” in the words “substitution/abasic probe GC contiguous sequence” or “substituted/abasic probe GC contiguous sequence” means that a sequence before substitution/becoming abasic is a sequence in which any one of guanine or cytosine is continuous for 3 or more bases.

Because guanine and cytosine are complementary, a probe complementary thereto also has a GC contiguous sequence in a case where a target nucleic acid has a GC contiguous sequence. In the present specification, the term “GC contiguous sequence” is used both when the sequence is present in a target nucleic acid and when the sequence is present in a probe. In particular, a GC contiguous sequence present in a target nucleic acid/sequence is referred to as a “sequence of any one of SEQ ID NOs: 1 to 10 in a target nucleic acid/sequence” or a “target GC contiguous sequence”. In addition, a GC contiguous sequence complementary to the target GC contiguous sequence present in the probe, in particular, a GC contiguous sequence which is complementary to a target GC contiguous sequence and is present in the probe fully complementary to a target sequence before cleavage, abasic site creation, or substitution, is referred to as a “probe GC contiguous sequence” or a “sequence portion complementary to any one sequence of SEQ ID NOs: 1 to 10 in a target sequence”. Such a probe sequence that is fully complementary to a target sequence before cleavage, abasic site creation, or substitution may be referred to as a “fully complementary probe sequence”. On the other hand, a probe sequence in which guanine or cytosine in the probe GC contiguous sequence becomes abasic or is substituted is referred to as a “substituted/abasic probe sequence”. A sequence corresponding to a probe GC contiguous sequence in the substituted/abasic probe sequence is referred to as a “substituted/abasic probe GC contiguous sequence”. For example, the probe of FIG. 1A shows a probe GC contiguous sequence in a fully complementary probe sequence, and the probe of FIG. 1B shows a substituted/abasic probe GC contiguous sequence in a substituted/abasic probe sequence.

In the present invention, the term “nucleobase” includes nucleotide analogs in addition to naturally occurring nucleotides. Naturally occurring nucleotides are deoxyribonucleotides or ribonucleotides having bases of adenine (A), guanine (G), cytosine (C), thymine (T), and/or uracil (U). Nucleotide analogues mean artificial nucleotides or nucleotide mimetics which have the same base as the naturally occurring deoxyribonucleotide or ribonucleotide described above, but in which a ribose chemical structure and/or a phosphodiester bond chemical structure is artificially modified. Examples thereof include glycol nucleic acid (GNA), bridged nucleic acid (BNA), 2′,4′-locked nucleic acid (LNA), peptide nucleic acid (PNA), threose nucleic acid (TNA), and morpholino nucleic acids. In the present specification, GNA, BNA, LNA, PNA, TNA, and morpholino nucleic acids may be interpreted as monomers or as polymers depending on context.

In the present invention, the term “polynucleobase” means a polymer compound in which the above-mentioned nucleobase is linearly polymerized. The polynucleobase may be a homopolymer composed of only one type of nucleobase (such as only a naturally occurring polynucleotide, or only a constituent unit of PNA). In addition, the polynucleobase may also be a copolymer of two or more types of nucleobases (such as a naturally occurring polynucleotide and PNA, or BNA and LNA). Accordingly, polynucleotides such as DNA and RNA, and polymers of GNA, BNA, LNA, PNA, TNA, and morpholino nucleic acids are also included in the polynucleobase. In the present specification, the polynucleobase may contain a pyrrole-imidazole polyamide (Peter B. Dervan et. Al., Nature (1998) 391-468; P. B. Dervan and R. W. Burli, Current Opinion in Chemical Biology 3 (1999) 688-693; P. B. Dervan, Bioorganic & Medicinal Chemistry 9 (2001) 215-2235.). In this case, abase in the present specification can be substituted with pyrrole and/or imidazole.

In the present specification, a “probe” and a “polynucleobase probe” are the same meaning, and mean a polynucleobase used to form a double strand by hybridization with a target sequence. The polynucleobase probe of the present invention includes a sequence in which at least one base in a probe GC contiguous sequence in a fully complementary probe sequence becomes abasic or is substituted (substituted/abasic probe sequence), or a sequence in which all bases from at least one base in the probe GC contiguous sequence to one end of a fully complementary probe are cleaved, as a portion that binds to a target sequence based on a fully complementary probe sequence complementary to a target sequence having at least one target GC contiguous sequence. In the present specification, the term “cleavage” means that a probe is designed such that only 2 or less bases of amino acids constituting the probe GC contiguous sequence are left at the end of the probe in the design stage of the probe. It is not necessary to perform “cleavage” in an actual production process of the probe. Accordingly, a “sequence cleaved to have, at the end, at least one sequence complementary to a sequence of 2 bases or less in any one sequence of SEQ ID NOs: 1 to 10 in the target sequence” means a sequence in which remaining bases excluding 2 or less bases located at the one end of a “probe GC contiguous sequence portion” contained in the fully complementary probe, and all bases up to one end of a “fully complementary probe” which are adjacent to the remaining bases are missing, or a sequence that contains, at the end of the probe, 2 or less bases derived from the end of the probe GC contiguous sequence portion.

When two or more target GC contiguous sequences are present in a target sequence, cleavage, substitution, or abasic site creation at a probe may be performed in any one of the probe GC contiguous sequences, or may be performed in two or more probe GC contiguous sequences. Preferably, the probe of the present invention is cleaved or substituted, or becomes abasic in the probe GC contiguous sequences at all locations. For example, when two or more target GC contiguous sequences are present in the target sequence, the probe of the present invention may be cleaved in any one of the probe GC contiguous sequences, and may be substituted or become abasic in the other probe GC contiguous sequence. Alternatively, when two or more target GC contiguous sequences are present in the target sequence, the probe of the present invention may be cleaved at two probe GC contiguous sequences, and when another probe GC contiguous sequence is present, the probe of the present invention may be substituted or become abasic at the probe GC contiguous sequence. Alternatively, when two or more target GC contiguous sequences are present in the target sequence, the probe of the present invention maybe substituted or become abasic in probe GC contiguous sequences at all locations. In other words, only one of cleavage, substitution, and abasic site creation may be used in probe GC contiguous sequences at all locations, or any one of cleavage, substitution, and abasic site creation is used for each of a plurality of probe GC contiguous sequences present in one probe so that cleavage, substitution, and abasic site creation are used in combination in one probe as a result. Furthermore, cleavage, substitution, and abasic site creation may be used in combination in one probe GC contiguous sequence. The probe of the present invention preferably does not have a probe GC contiguous sequence as a result of cleavage, substitution, or abasic site creation in probe GC contiguous sequences at all locations in an initially selected fully complementary probe sequence.

Furthermore, the probe or the polynucleobase probe of the present invention may have a portion that does not bind to a target sequence in addition to the above-described portion that binds to the target sequence. Such a portion that does not bind to the target sequence may be a label or a linker, maybe bound to another molecule, or maybe used for the purpose of improving stability. For example, such a portion that does not bind to the target sequence may contain a polynucleobase that is not complementary to the target sequence such as a tag sequence or a linker sequence, or may be bound a low molecular weight compound or proteins. In one example, a portion that does not bind to the target sequence is “modification” to be described later.

In the present specification, the phrase “becoming abasic” means that no base portion is present in a nucleobase. In an abasic site of DNA or RNA, no base is bound to the 1′ position of the sugar, but a hydroxyl group, a hydrogen atom, a lower acyl group (such as an acetyl group), or a lower alkyl group (such as a methyl group) is bound thereto. In a case of an abasic site of PNA, a substituent of a methyl carbonyl group bound to tertiary amine of a glycine skeleton is a hydroxyl group, a hydrogen atom, a lower acyl group (such as an acetyl group), or a lower alkyl group (such as a methyl group) instead of a base. Alternatively, in the case of an abasic site of PNA, a nitrogen atom of a glycine skeleton is substituted with a carbon atom (which may have a lower acyl group (such as an acetyl group) or a lower alkyl group (such as a methyl group) as a substituent).

In addition, in the present specification, a case in which a base is “substituted” includes a case in which a base portion in a nucleobase is substituted with a non-complementary base. In addition, a case in which a base in a probe sequence is “substituted” includes a case in which a base portion in a nucleobase is substituted with a group other than adenine, guanine, cytosine, uracil, and thymine (for example, a phenyl group, an anthraquinone group, and the like). A group introduced by base substitution is preferably a group that does not inhibit double-strand formation with a target sequence by another non-substituted base in the probe sequence.

A position of a base to become abasic or be substituted in the probe GC contiguous sequence is not particularly limited. For example, a base may be in the inside of the probe GC contiguous sequence (such as G*G and C*CC. Herein, “*” represents a base to become abasic or be substituted. The same applies in the present specification), or at the end (such as GG * and *CC). Preferably, guanine or cytosine in the middle of the inside of the probe GC contiguous sequence becomes abasic or is substituted (such as C*C and GG*GG). For example, in a case where the probe GC contiguous sequence is GGG, G*G is preferable, and similarly, in a case of CCC, C*C is preferable. In addition, preferably, the GC contiguous sequence after substitution or becoming abasic does not have a sequence in which G or C is continuous for 3 or more bases.

The number of bases to become abasic or be substituted in the probe GC contiguous sequence is not particularly limited as long as binding power with a target sequence is maintained. For example, a ratio may be 2 or 3 bases to 6 to 7 guanines or cytosines, 1 or 2 bases to 3 to 5 guanines or cytosines, 1 base to 3 to 4 guanines or cytosines, or 1 base to 3 guanines or cytosines.

Examples of substituted/abasic probe GC contiguous sequences include G*GG*GG (SEQ ID NO: 11), GG*G*GG (SEQ ID NO: 12), GG*GG*G (SEQ ID NO: 13), *G*G*GG (SEQ ID NO: 14), *G*GG*G (SEQ ID NO: 15), *GG*G*G (SEQ ID NO: 16), *GG*GG* (SEQ ID NO: 17), G*G*G*G (SEQ ID NO: 18), G*GG*G* (SEQ ID NO: 19), G*G*GG* (SEQ ID NO: 20), GG*G*G* (SEQ ID NO: 21), C*CC*CC (SEQ ID NO: 22), CC*C*CC (SEQ ID NO: 23), CC*CC*C (SEQ ID NO: 24), *C*C*CC (SEQ ID NO: 25), *C*CC*C (SEQ ID NO: 26), *CC*C*C (SEQ ID NO: 27), *CC*CC* (SEQ ID NO 28), C*C*C*C (SEQ ID NO 29), C*C*CC* (SEQ ID NO: 30), C*CC*C* (SEQ ID NO: 31), CC*C*C* (SEQ ID NO: 32), *GG*GG (SEQ ID NO: 33), G*G*GG (SEQ ID NO: 34), G*GG*G (SEQ ID NO: 35), GG*G*G (SEQ ID NO: 36), GG*GG* (SEQ ID NO: 37), *G*G*G (SEQ ID NO: 38), *G*GG* (SEQ ID NO: 39), *GG*G* (SEQ ID NO: 40), G*G*G* (SEQ ID NO: 41), *CC*CC (SEQ ID NO: 42), C*C*CC (SEQ ID NO: 43), C*CC*C (SEQ ID NO: 44), CC*C*C (SEQ ID NO: 45), CC*CC* (SEQ ID NO: 46), *C*C*C (SEQ ID NO: 47), *C*CC* (SEQ ID NO: 48), *CC*C* (SEQ ID NO: 49), C*C*C* (SEQ ID NO: 50), GG*GG (SEQ ID NO: 51), *G*GG (SEQ ID NO: 52), *GG*G (SEQ ID NO: 53), G*G*G (SEQ ID NO: 54), G*GG* (SEQ ID NO: 55), GG*G* (SEQ ID NO: 56), CC*CC (SEQ ID NO: 57), *C*CC (SEQ ID NO: 58), *CC*C (SEQ ID NO: 59), C*C*C (SEQ ID NO: 60), C*CC* (SEQ ID NO: 61), CC*C* (SEQ ID NO: 62), G*GG (SEQ ID NO: 63), GG*G (SEQ ID NO: 64), *G*G (SEQ ID NO: 65), *GG* (SEQ ID NO: 66), G*G* (SEQ ID NO: 67), C*CC (SEQ ID NO: 68), CC*C (SEQ ID NO: 69), *C*C (SEQ ID NO: 70), *CC* (SEQ ID NO: 71), C*C* (SEQ ID NO: 72), *GG (SEQ ID NO: 73), G*G (SEQ ID NO: 74), GG* (SEQ ID NO: 75), *G* (SEQ ID NO: 76), *CC (SEQ ID NO: 77), C*C (SEQ ID NO: 78), CC* (SEQ ID NO: 79), and *C* (SEQ ID NO: 80).

In the polynucleobase probe of the present invention, a chain length of a portion binding to a target sequence is a length such that a binding rate (a false positive rate) with a nucleic acid having a sequence other than a target sequence is increased due to the presence of a probe GC contiguous sequence, and is specifically is 10- to 50-mer. For example, a chain length of a portion binding to a target sequence in the polynucleobase probe of the present invention can be 10-mer or more, 11-mer or more, 12-mer or more, 13-mer or more, 14-mer or more, 15-mer or more, 16-mer or more, 17-mer or more, or 18-mer or more.

In addition, a chain length of a portion binding to a target sequence in the polynucleobase probe of the present invention can be 50-mer or less, 45-mer or less, 40-mer or less, 35-mer or less, 30-mer or less, 29-mer or less, 28-mer or less, 27-mer or less, 26-mer or less, or 25-mer or less.

For example, a chain length of a portion binding to a target sequence in the polynucleobase probe of the present invention can be 10- to 40-mer, 13- to 30-mer, 15- to 28-mer, or 18- to 25-mer.

The above-mentioned “chain length of a portion binding to a target sequence in the polynucleobase probe of the present invention” may be read as a chain length of the polynucleobase probe.

The nucleobase probe in the present specification may be appropriately “modified”. For example, the modification includes a label for detection, a functional group for binding, and the like. Any label can be used without particular limitation as long as it can be used in the field of nucleic acid detection. For example, various methods such as radioactive substance (RI), enzyme (biotin and the like), hapten (digoxigenin (DIG) and the like), affinity tag, and fluorescent colorants are known.

As fluorescent colorants, various types of red, orange, yellow, green, blue, and purple are known. It is possible to use dansyl, TRITC, fluorescein, rhodamine, Texas red, IAEDANS, cyanine dyes (Cy3, Cy3.5, Cy5, Cy5.5, Cy7), Hoechst, BFP, CFP, WGFP, GFP, YFP, RFP, EGFP, FITC, AlexaFluor, tdTomato, TRITC, TXRED, mCherry-A, mCherry-C, and the like.

In addition, the probe of the present invention may be immobilized on a solid phase. For example, the probe may be bound to an array, a bead, or a chip.

A “functional group for binding” is not particularly limited as long as it is a group used for binding the nucleobase probe in the present specification to a solid phase or another substance, and examples thereof include a hydroxyl group, a halogen atom, an amino group, an amido group, an imide group, a guanidine group, a urea group, an alkene, an alkyne, a sulfonic acid, a carboxylic acid group, an ester group, and the like.

The term “target nucleic acid” in the present specification means a nucleic acid having a GC contiguous sequence, which is a target nucleic acid of which the presence is to be detected or quantitatively determined by the probe of the present invention. For example, in a case where the probe of the present invention is used for the purpose of diagnosing a disease or disorder, a target nucleic acid means DNA or RNA derived from a living body. A target nucleic acid may have 1 to 5, 1 to 4, 1 to 3, 1 to 2, or 1 GC contiguous sequence.

A length of the target nucleic acid is not particularly limited, but a target nucleic acid of 50-mer or less, 45-mer or less, 40-mer or less, 35-mer or less, 30-mer or less, 29-mer or less, 28-mer or less, 27-mer or less, 26-mer or less, or 25-mer or less are preferable from the viewpoint of the probe of the present invention being particularly effective for double-strand formation with a target nucleic acid for which a probe avoiding a target GC contiguous sequence cannot be designed. For example, a chain length of the target nucleic acid can be 10-mer or more, 11-mer or more, 12-mer or more, 13-mer or more, 14-mer or more, 15-mer or more, 16-mer or more, 17-mer or more, or 18-mer or more. As an example, a chain length of the target nucleic acid is 10- to 50-mer, 10- to 40-mer, 13- to 30-mer, 15- to 28-mer, or 18- to 25-mer.

A representative example of target nucleic acids includes an miRNA having a GC contiguous sequence. Examples of such sequences include sequences described in the following table. In the table, the underline indicates a GC contiguous sequence. The numerical values beside the sequence represent, in order, a full length of the sequence, the number of target GC contiguous sequences contained in the target nucleic acid, and a length of the longest target GC contiguous sequence contained in the target nucleic acid.

Examples of target sequences having GC contiguous sequences or sequences complementary to the target sequences or miRNAs are as follows:

hsa-miR-3676-5p:
(SEQ ID NO: 81)
AGGAGAUCCUGGGUU,
hsa-miR-4279:
(SEQ ID NO: 82)
CUCUCCUCCCGGCUUC,
hsa-miR-4310:
(SEQ ID NO: 83)
GCAGCAUUCAUGUCCC,
hsa-miR-4261:
(SEQ ID NO: 84)
AGGAAACAGGGACCCA,
hsa-miR-1281:
(SEQ ID NO: 85)
UCGCCUCCUCCUCUCCC,
hsa-miR-3201:
(SEQ ID NO: 86)
GGGAUAUGAAGAAAAAU,
hsa-miR-4251:
(SEQ ID NO: 87)
CCUGAGAAAAGGGCCAA,
hsa-miR-4296:
(SEQ ID NO: 88)
AUGUGGGCUCAGGCUCA,
hsa-miR-4304:
(SEQ ID NO: 89)
CCGGCAUGUCCAGGGCA,
hsa-miR-4317:
(SEQ ID NO: 90)
ACAUUGCCAGGGAGUUU,
hsa-miR-4318:
(SEQ ID NO: 91)
CACUGUGGGUACAUGCU,
hsa-miR-4319:
(SEQ ID NO: 92)
UCCCUGAGCAAAGCCAC,
hsa-miR-4328:
(SEQ ID NO: 93)
CCAGUUUUCCCAGGAUU,
hsa-miR-4419a:
(SEQ ID NO: 94)
UGAGGGAGGAGACUGCA,
hsa-miR-4441:
(SEQ ID NO: 95)
ACAGGGAGGAGAUUGUA,
hsa-miR-4442:
(SEQ ID NO: 96)
GCCGGACAAGAGGGAGG,
hsa-miR-4443:
(SEQ ID NO: 97)
UUGGAGGCGUGGGUUUU,
hsa-miR-4455:
(SEQ ID NO: 98)
AGGGUGUGUGUGUUUUU,
hsa-miR-4481:
(SEQ ID NO: 99)
GGAGUGGGCUGGUGGUU,
hsa-miR-4486:
(SEQ ID NO: 100)
GCUGGGCGAGGCUGGCA,
hsa-miR-4499:
(SEQ ID NO: 101)
AAGACUGAGAGGAGGGA,
hsa-miR-4535:
(SEQ ID NO: 102)
GUGGACCUGGCUGGGAC,
hsa-miR-1274b:
(SEQ ID NO: 103)
UCCCUGUUCGGGCGCCA,
hsa-miR-1280:
(SEQ ID NO: 104)
UCCCACCGCUGCCACCC,
hsa-miR-4294:
(SEQ ID NO: 105)
GGGAGUCUACAGCAGGG,
hsa-miR-4497:
(SEQ ID NO: 106)
CUCCGGGACGGCUGGGC,
hsa-miR-3195:
(SEQ ID NO: 107)
CGCGCCGGGCCCGGGUU,
hsa-miR-1207-3p:
(SEQ ID NO: 108)
UCAGCUGGCCCUCAUUUC,
hsa-miR-1260:
(SEQ ID NO: 109)
AUCCCACCUCUGCCACCA,
hsa-miR-1274a:
(SEQ ID NO: 110)
GUCCCUGUUCAGGCGCCA,
hsa-miR-1308:
(SEQ ID NO: 111)
GCAUGGGUGGUUCAGUGG,
hsa-miR-1321:
(SEQ ID NO: 112)
CAGGGAGGUGAAUGUGAU,
hsa-miR-1825:
(SEQ ID NO: 113)
UCCAGUGCCCUCCUCUCC,
hsa-miR-3155b:
(SEQ ID NO: 114)
CCAGGCUCUGCAGUGGGA,
hsa-miR-4300:
(SEQ ID NO: 115)
UGGGAGCUGGACUACUUC,
hsa-miR-4308:
(SEQ ID NO: 116)
UCCCUGGAGUUUCUUCUU,
hsa-miR-4320:
(SEQ ID NO: 117)
GGGAUUCUGUAGCUUCCU,
hsa-miR-4519:
(SEQ ID NO: 118)
CAGCAGUGCGCAGGGCUG,
hsa-miR-5587-5p:
(SEQ ID NO: 119)
AUGGUCACCUCCGGGACU,
hsa-miR-5703:
(SEQ ID NO: 120)
AGGAGAAGUCGGGAAGGU,
hsa-miR-6126:
(SEQ ID NO: 121)
GUGAAGGCCCGGCGGAGA,
mmu-miR-696:
(SEQ ID NO: 122)
GCGUGUGCUUGCUGUGGG,
hsa-miR-4314:
(SEQ ID NO: 123)
CUCUGGGAAAUGGGACAG,
hsa-miR-4505:
(SEQ ID NO: 124)
AGGCUGGGCUGGGACGGA,
hsa-miR-4530:
(SEQ ID NO: 125)
CCCAGCAGGACGGGAGCG,
hsa-miR-4710:
(SEQ ID NO: 126)
GGGUGAGGGCAGGUGGUU,
hsa-miR-4417:
(SEQ ID NO: 127)
GGUGGGCUUCCCGGAGGG,
hsa-miR-1224-5p:
(SEQ ID NO: 128)
GUGAGGACUCGGGAGGUGG,
hsa-miR-1290:
(SEQ ID NO: 129)
UGGAUUUUUGGAUCAGGGA,
hsa-miR-3176:
(SEQ ID NO: 130)
ACUGGCCUGGGACUACCGG,
hsa-miR-3649:
(SEQ ID NO: 131)
AGGGACCUGAGUGUCUAAG,
hsa-miR-4289:
(SEQ ID NO: 132)
GCAUUGUGCAGGGCUAUCA,
hsa-miR-4329:
(SEQ ID NO: 133)
CCUGAGACCCUAGUUCCAC,
hsa-miR-4487:
(SEQ ID NO: 134)
AGAGCUGGCUGAAGGGCAG,
hsa-miR-4736:
(SEQ ID NO: 135)
AGGCAGGUUAUCUGGGCUG,
hsa-miR-5588-3p:
(SEQ ID NO: 136)
AAGUCCCACUAAUGCCAGC,
hsa-miR-585:
(SEQ ID NO: 137)
UGGGCGUAUCUGUAUGCUA,
hsa-miR-6070:
(SEQ ID NO: 138)
CCGGUUCCAGUCCCUGGAG,
hsa-miR-6130:
(SEQ ID NO: 139)
UGAGGGAGUGGAUUGUAUG,
hsa-miR-6131:
(SEQ ID NO: 140)
GGCUGGUCAGAUGGGAGUG,
hsa-miR-632:
(SEQ ID NO: 141)
GUGUCUGCUUCCUGUGGGA,
hsa-miR-648:
(SEQ ID NO: 142)
AAGUGUGCAGGGCACUGGU,
mmu-miR-684:
(SEQ ID NO: 143)
AGUUUUCCCUUCAAGUCAA,
mmu-miR-698:
(SEQ ID NO: 144)
CAUUCUCGUUUCCUUCCCU,
hsa-miR-3141:
(SEQ ID NO: 145)
GAGGGCGGGUGGAGGAGGA,
hsa-miR-3181:
(SEQ ID NO: 146)
AUCGGGCCCUCGGCGCCGG,
hsa-miR-4265:
(SEQ ID NO: 147)
CUGUGGGCUCAGCUCUGGG,
hsa-miR-4287:
(SEQ ID NO: 148)
UCUCCCUUGAGGGCACUUU,
hsa-miR-4290:
(SEQ ID NO: 149)
UGCCCUCCUUUCUUCCCUC,
hsa-miR-6080:
(SEQ ID NO: 150)
UCUAGUGCGGGCGUUCCCG,
hsa-miR-6129:
(SEQ ID NO: 151)
UGAGGGAGUUGGGUGUAUA,
hsa-miR-6133:
(SEQ ID NO: 152)
UGAGGGAGGAGGUUGGGUA,
hsa-miR-6127:
(SEQ ID NO: 153)
UGAGGGAGUGGGUGGGAGG,
rno-miR-347:
(SEQ ID NO: 154)
UGUCCCUCUGGGUCGCCCA,
hsa-miR-1205:
(SEQ ID NO: 155)
UCUGCAGGGUUUGCUUUGAG,
hsa-miR-1231:
(SEQ ID NO: 156)
GUGUCUGGGCGGACAGCUGC,
hsa-miR-1276:
(SEQ ID NO: 157)
UAAAGAGCCCUGUGGAGACA,
hsa-miR-1976:
(SEQ ID NO: 158)
CCUCCUGCCCUCCUUGCUGU,
hsa-miR-3115:
(SEQ ID NO: 159)
AUAUGGGUUUACUAGUUGGU,
hsa-miR-3622b-5p:
(SEQ ID NO: 160)
AGGCAUGGGAGGUCAGGUGA,
hsa-miR-3917:
(SEQ ID NO: 161)
GCUCGGACUGAGCAGGUGGG,
hsa-miR-4301:
(SEQ ID NO: 162)
UCCCACUACUUCACUUGUGA,
hsa-miR-4326:
(SEQ ID NO: 163)
UGUUCCUCUGUCUCCCAGAC,
hsa-miR-4429:
(SEQ ID NO: 164)
AAAAGCUGGGCUGAGAGGCG,
hsa-miR-4485:
(SEQ ID NO: 165)
UAACGGCCGCGGUACCCUAA,
hsa-miR-4506:
(SEQ ID NO: 166)
AAAUGGGUGGUCUGAGGCAA,
hsa-miR-4784:
(SEQ ID NO: 167)
UGAGGAGAUGCUGGGACUGA,
hsa-miR-490-5p:
(SEQ ID NO: 168)
CCAUGGAUCUCCAGGUGGGU,
hsa-miR-572:
(SEQ ID NO: 169)
GUCCGCUCGGCGGUGGCCCA,
hsa-miR-591:
(SEQ ID NO: 170)
AGACCAUGGGUUCUCAUUGU,
hsa-miR-6083:
(SEQ ID NO: 171)
CUUAUAUCAGAGGCUGUGGG,
hsa-miR-609:
(SEQ ID NO: 172)
AGGGUGUUUCUCUCAUCUCU,
hsa-miR-6722-5p:
(SEQ ID NO: 173)
AGGCGCACCCGACCACAUGC,
hsa-miR-877:
(SEQ ID NO: 174)
GUAGAGGAGAUGGCGCAGGG,
mmu-miR-805:
(SEQ ID NO: 175)
GAAUUGAUCAGGACAUAGGG,
hsa-miR-1233:
(SEQ ID NO: 176)
UGAGCCCUGUCCUCCCGCAG,
hsa-miR-202:
(SEQ ID NO: 177)
AGAGGUAUAGGGCAUGGGAA,
hsa-miR-326:
(SEQ ID NO: 178)
CCUCUGGGCCCUUCCUCCAG,
hsa-miR-4324:
(SEQ ID NO: 179)
CCCUGAGACCCUAACCUUAA,
hsa-miR-4648:
(SEQ ID NO: 180)
UGUGGGACUGCAAAUGGGAG,
hsa-miR-4701-3p:
(SEQ ID NO: 181)
AUGGGUGAUGGGUGUGGUGU,
hsa-miR-6086:
(SEQ ID NO: 182)
GGAGGUUGGGAAGGGCAGAG,
mmu-miR-343:
(SEQ ID NO: 183)
UCUCCCUUCAUGUGCCCAGA,
hsa-miR-4507:
(SEQ ID NO: 184)
CUGGGUUGGGCUGGGCUGGG,
gga-miR-757:
(SEQ ID NO: 185)
GCAGAGCUGCAGAUGGGAUUC,
hsa-miR-1178:
(SEQ ID NO: 186)
UUGCUCACUGUUCUUCCCUAG,
hsa-miR-1181:
(SEQ ID NO: 187)
CCGUCGCCGCCACCCGAGCCG,
hsa-miR-1185:
(SEQ ID NO: 188)
AGAGGAUACCCUUUGUAUGUU,
hsa-miR-1203:
(SEQ ID NO: 189)
CCCGGAGCCAGGAUGCAGCUC,
hsa-miR-1257:
(SEQ ID NO: 190)
AGUGAAUGAUGGGUUCUGACC,
hsa-miR-1286:
(SEQ ID NO: 191)
UGCAGGACCAAGAUGAGCCCU,
hsa-miR-1288:
(SEQ ID NO: 192)
UGGACUGCCCUGAUCUGGAGA,
hsa-miR-1295:
(SEQ ID NO: 193)
UUAGGCCGCAGAUCUGGGUGA,
hsa-miR-129-5p:
(SEQ ID NO: 194)
CUUUUUGCGGUCUGGGCUUGC,
hsa-miR-1302:
(SEQ ID NO: 195)
UUGGGACAUACUUAUGCUAAA,
hsa-miR-1302:
(SEQ ID NO: 196)
UUGGGACAUACUUAUGCUAAA,
hsa-miR-1302:
(SEQ ID NO: 197)
UUGGGACAUACUUAUGCUAAA,
hsa-miR-1302:
(SEQ ID NO: 198)
UUGGGACAUACUUAUGCUAAA,
hsa-miR-1302:
(SEQ ID NO: 199)
UUGGGACAUACUUAUGCUAAA,
hsa-miR-130b*:
(SEQ ID NO: 200)
ACUCUUUCCCUGUUGCACUAC,
hsa-miR-140-3p:
(SEQ ID NO: 201)
UACCACAGGGUAGAACCACGG,
hsa-miR-1909*:
(SEQ ID NO: 202)
UGAGUGCCGGUGCCUGCCCUG,
hsa-miR-190b:
(SEQ ID NO: 203)
UGAUAUGUUUGAUAUUGGGUU,
hsa-miR-21*:
(SEQ ID NO: 204)
CAACACCAGUCGAUGGGCUGU,
hsa-miR-2114*:
(SEQ ID NO: 205)
CGAGCCUCAAGCAAGGGACUU,
hsa-miR-222:
(SEQ ID NO: 206)
AGCUACAUCUGGCUACUGGGU,
hsa-miR-2277-3p:
(SEQ ID NO: 207)
UGACAGCGCCCUGCCUGGCUC,
hsa-miR-23a:
(SEQ ID NO: 208)
AUCACAUUGCCAGGGAUUUCC,
hsa-miR-23b:
(SEQ ID NO: 209)
AUCACAUUGCCAGGGAUUACC,
hsa-miR-25*:
(SEQ ID NO: 210)
AGGCGGAGACUUGGGCAAUUG,
hsa-miR-2909:
(SEQ ID NO: 211)
GUUAGGGCCAACAUCUCUUGG,
hsa-miR-3124-5p:
(SEQ ID NO: 212)
UUCGCGGGCGAAGGCAAAGUC,
hsa-miR-3130-3p:
(SEQ ID NO: 213)
GCUGCACCGGAGACUGGGUAA,
hsa-miR-3130-5p:
(SEQ ID NO: 214)
UACCCAGUCUCCGGUGCAGCC,
hsa-miR-3155a:
(SEQ ID NO: 215)
CCAGGCUCUGCAGUGGGAACU,
hsa-miR-3156-3p:
(SEQ ID NO: 216)
CUCCCACUUCCAGAUCUUUCU,
hsa-miR-3158-5p:
(SEQ ID NO: 217)
CCUGCAGAGAGGAAGCCCUUC,
hsa-miR-3194-5p:
(SEQ ID NO: 218)
GGCCAGCCACCAGGAGGGCUG,
hsa-miR-3622b-3p:
(SEQ ID NO: 219)
UCACCUGAGCUCCCGUGCCUG,
hsa-miR-3657:
(SEQ ID NO: 220)
UGUGUCCCAUUAUUGGUGAUU,
hsa-miR-3659:
(SEQ ID NO: 221)
UGAGUGUUGUCUACGAGGGCA,
hsa-miR-3918:
(SEQ ID NO: 222)
ACAGGGCCGCAGAUGGAGACU,
hsa-miR-4269:
(SEQ ID NO: 223)
GCAGGCACAGACAGCCCUGGC,
hsa-miR-4321:
(SEQ ID NO: 224)
UUAGCGGUGGACCGCCCUGCG,
hsa-miR-4422:
(SEQ ID NO: 225)
AAAAGCAUCAGGAAGUACCCA,
hsa-miR-4523:
(SEQ ID NO: 226)
GACCGAGAGGGCCUCGGCUGU,
hsa-miR-4529-3p:
(SEQ ID NO: 227)
AUUGGACUGCUGAUGGCCCGU,
hsa-miR-455-3p:
(SEQ ID NO: 228)
GCAGUCCAUGGGCAUAUACAC,
hsa-miR-4635:
(SEQ ID NO: 229)
UCUUGAAGUCAGAACCCGCAA,
hsa-miR-4690-3p:
(SEQ ID NO: 230)
GCAGCCCAGCUGAGGCCUCUG,
hsa-miR-4717-3p:
(SEQ ID NO: 231)
ACACAUGGGUGGCUGUGGCCU,
hsa-miR-4732-3p:
(SEQ ID NO: 232)
GCCCUGACCUGUCCUGUUCUG,
hsa-miR-4746-3p:
(SEQ ID NO: 233)
AGCGGUGCUCCUGCGGGCCGA,
hsa-miR-4761-3p:
(SEQ ID NO: 234)
GAGGGCAUGCGCACUUUGUCC,
hsa-miR-4804-3p:
(SEQ ID NO: 235)
UGCUUAACCUUGCCCUCGAAA,
hsa-miR-483-3p:
(SEQ ID NO: 236)
UCACUCCUCUCCUCCCGUCUU,
hsa-miR-488*:
(SEQ ID NO: 237)
CCCAGAUAAUGGCACUCUCAA,
hsa-miR-5006-3p:
(SEQ ID NO: 238)
UUUCCCUUUCCAUCCUGGCAG,
hsa-miR-5006-5p:
(SEQ ID NO: 239)
UUGCCAGGGCAGGAGGUGGAA,
hsa-miR-502-5p:
(SEQ ID NO: 240)
AUCCUUGCUAUCUGGGUGCUA,
hsa-miR-506:
(SEQ ID NO: 241)
UAAGGCACCCUUCUGAGUAGA,
hsa-miR-5089-5p:
(SEQ ID NO: 242)
GUGGGAUUUCUGAGUAGCAUC,
hsa-miR-5571-5p:
(SEQ ID NO: 243)
CAAUUCUCAAAGGAGCCUCCC,
hsa-miR-5588-5p:
(SEQ ID NO: 244)
ACUGGCAUUAGUGGGACUUUU,
hsa-miR-5589-5p:
(SEQ ID NO: 245)
GGCUGGGUGCUCUUGUGCAGU,
hsa-miR-5694:
(SEQ ID NO: 246)
CAGAUCAUGGGACUGUCUCAG,
hsa-miR-583:
(SEQ ID NO: 247)
CAAAGAGGAAGGUCCCAUUAC,
hsa-miR-588:
(SEQ ID NO: 248)
UUGGCCACAAUGGGUUAGAAC,
hsa-miR-6075:
(SEQ ID NO: 249)
ACGGCCCAGGCGGCAUUGGUG,
hsa-miR-6077:
(SEQ ID NO: 250)
GGGAAGAGCUGUACGGCCUUC,
hsa-miR-610:
(SEQ ID NO: 251)
UGAGCUAAAUGUGUGCUGGGA,
hsa-miR-631:
(SEQ ID NO: 252)
AGACCUGGCCCAGACCUCAGC,
hsa-miR-6500-3p:
(SEQ ID NO: 253)
ACACUUGUUGGGAUGACCUGC,
hsa-miR-6503-3p:
(SEQ ID NO: 254)
GGGACUAGGAUGCAGACCUCC,
hsa-miR-6507-5p:
(SEQ ID NO: 255)
GAAGAAUAGGAGGGACUUUGU,
hsa-miR-6508-5p:
(SEQ ID NO: 256)
UCUAGAAAUGCAUGACCCACC,
hsa-miR-6513-3p:
(SEQ ID NO: 257)
UCAAGUGUCAUCUGUCCCUAG,
hsa-miR-6515-5p:
(SEQ ID NO: 258)
UUGGAGGGUGUGGAAGACAUC,
hsa-miR-652:
(SEQ ID NO: 259)
AAUGGCGCCACUAGGGUUGUG,
hsa-miR-671-3p:
(SEQ ID NO: 260)
UCCGGUUCUCAGGGCUCCACC,
hsa-miR-6718-5p:
(SEQ ID NO: 261)
UAGUGGUCAGAGGGCUUAUGA,
mmu-miR-700:
(SEQ ID NO: 262)
CACGCGGGAACCGAGUCCACC,
mmu-miR-714:
(SEQ ID NO: 263)
CGACGAGGGCCGGUCGGUCGC,
rno-miR-336:
(SEQ ID NO: 264)
UCACCCUUCCAUAUCUAGUCU,
hsa-miR-1539:
(SEQ ID NO: 265)
UCCUGCGCGUCCCAGAUGCCC,
hsa-miR-188-3p:
(SEQ ID NO: 266)
CUCCCACAUGCAGGGUUUGCA,
hsa-miR-188-5p:
(SEQ ID NO: 267)
CAUCCCUUGCAUGGUGGAGGG,
hsa-miR-2116*:
(SEQ ID NO: 268)
CCUCCCAUGCCAAGAACUCCC,
hsa-miR-4437:
(SEQ ID NO: 269)
UGGGCUCAGGGUACAAAGGUU,
hsa-miR-4674:
(SEQ ID NO: 270)
CUGGGCUCGGGACGCGCGGCU,
hsa-miR-4725-5p:
(SEQ ID NO: 271)
AGACCCUGCAGCCUUCCCACC,
hsa-miR-4763-5p:
(SEQ ID NO: 272)
CGCCUGCCCAGCCCUCCUGCU,
hsa-miR-5008-3p:
(SEQ ID NO: 273)
CCUGUGCUCCCAGGGCCUCGC,
hsa-miR-5196-3p:
(SEQ ID NO: 274)
UCAUCCUCGUCUCCCUCCCAG,
hsa-miR-5591-3p:
(SEQ ID NO: 275)
AUACCCAUAGCUUAGCUCCCA,
hsa-miR-5591-5p:
(SEQ ID NO: 276)
UGGGAGCUAAGCUAUGGGUAU,
hsa-miR-596:
(SEQ ID NO: 277)
AAGCCUGCCCGGCUCCUCGGG,
hsa-miR-629:
(SEQ ID NO: 278)
UGGGUUUACGUUGGGAGAACU,
hsa-miR-662:
(SEQ ID NO: 279)
UCCCACGUUGUGGCCCAGCAG,
hsa-miR-886-3p:
(SEQ ID NO: 280)
CGCGGGUGCUUACUGACCCUU,
mmu-miR-712:
(SEQ ID NO: 281)
CUCCUUCACCCGGGCGGUACC,
mmu-miR-718:
(SEQ ID NO: 282)
CUUCCGCCCGGCCGGGUGUCG,
hsa-miR-4646-3p:
(SEQ ID NO: 283)
AUUGUCCCUCUCCCUUCCCAG,
hsa-miR-4783-5p:
(SEQ ID NO: 284)
GGCGCGCCCAGCUCCCGGGCU,
hsa-let-7b*:
(SEQ ID NO: 285)
CUAUACAACCUACUGCCUUCCC,
hsa-let-7f-1*:
(SEQ ID NO: 286)
CUAUACAAUCUAUUGCCUUCCC,
hsa-miR-100:
(SEQ ID NO: 287)
AACCCGUAGAUCCGAACUUGUG,
hsa-miR-106b*:
(SEQ ID NO: 288)
CCGCACUGUGGGUACUUGCUGC,
hsa-miR-1180:
(SEQ ID NO: 289)
UUUCCGGCUCGCGUGGGUGUGU,
hsa-miR-125a-3p:
(SEQ ID NO: 290)
ACAGGUGAGGUUCUUGGGAGCC,
hsa-miR-125b-2*:
(SEQ ID NO: 291)
UCACAAGUCAGGCUCUUGGGAC,
hsa-miR-1262:
(SEQ ID NO: 292)
AUGGGUGAAUUUGUAGAAGGAU,
hsa-miR-1263:
(SEQ ID NO: 293)
AUGGUACCCUGGCAUACUGAGU,
hsa-miR-127-5p:
(SEQ ID NO: 294)
CUGAAGCUCAGAGGGCUCUGAU,
hsa-miR-1284:
(SEQ ID NO: 295)
UCUAUACAGACCCUGGCUUUUC,
hsa-miR-1285:
(SEQ ID NO: 296)
UCUGGGCAACAAAGUGAGACCU,
hsa-miR-1293:
(SEQ ID NO: 297)
UGGGUGGUCUGGAGAUUUGUGC,
hsa-miR-1299:
(SEQ ID NO: 298)
UUCUGGAAUUCUGUGUGAGGGA,
hsa-miR-1305:
(SEQ ID NO: 299)
UUUUCAACUCUAAUGGGAGAGA,
hsa-miR-130a:
(SEQ ID NO: 300)
CAGUGCAAUGUUAAAAGGGCAU,
hsa-miR-130b:
(SEQ ID NO: 301)
CAGUGCAAUGAUGAAAGGGCAU,
hsa-miR-135a*:
(SEQ ID NO: 302)
UAUAGGGAUUGGAGCCGUGGCG,
hsa-miR-138-1*:
(SEQ ID NO: 303)
GCUACUUCACAACACCAGGGCC,
hsa-miR-138-2*:
(SEQ ID NO: 304)
GCUAUUUCACGACACCAGGGUU,
hsa-miR-139-3p:
(SEQ ID NO: 305)
GGAGACGCGGCCCUGUUGGAGU,
hsa-miR-140-5p:
(SEQ ID NO: 306)
CAGUGGUUUUACCCUAUGGUAG,
hsa-miR-146a:
(SEQ ID NO: 307)
UGAGAACUGAAUUCCAUGGGUU,
hsa-miR-146b-3p:
(SEQ ID NO: 308)
UGCCCUGUGGACUCAGUUCUGG,
hsa-miR-1471:
(SEQ ID NO: 309)
GCCCGCGUGUGGAGCCAGGUGU,
hsa-miR-183*:
(SEQ ID NO: 310)
GUGAAUUACCGAAGGGCCAUAA,
hsa-miR-184:
(SEQ ID NO: 311)
UGGACGGAGAACUGAUAAGGGU,
hsa-miR-186:
(SEQ ID NO: 312)
CAAAGAAUUCUCCUUUUGGGCU,
hsa-miR-1912:
(SEQ ID NO: 313)
UACCCAGAGCAUGCAGUGUGAA,
hsa-miR-193a-3p:
(SEQ ID NO: 314)
AACUGGCCUACAAAGUCCCAGU,
hsa-miR-196a:
(SEQ ID NO: 315)
UAGGUAGUUUCAUGUUGUUGGG,
hsa-miR-196b:
(SEQ ID NO: 316)
UAGGUAGUUUCCUGUUGUUGGG,
hsa-miR-197:
(SEQ ID NO: 317)
UUCACCACCUUCUCCACCCAGC,
hsa-miR-1977:
(SEQ ID NO: 318)
GAUUAGGGUGCUUAGCUGUUAA,
hsa-miR-1979:
(SEQ ID NO: 319)
CUCCCACUGCUUCACUUGACUA,
hsa-miR-200b*:
(SEQ ID NO: 320)
CAUCUUACUGGGCAGCAUUGGA,
hsa-miR-2000*:
(SEQ ID NO: 321)
CGUCUUACCCAGCAGUGUUUGG,
hsa-miR-204:
(SEQ ID NO: 322)
UUCCCUUUGUCAUCCUAUGCCU,
hsa-miR-20b*:
(SEQ ID NO: 323)
ACUGUAGUAUGGGCACUUCCAG,
hsa-miR-211:
(SEQ ID NO: 324)
UUCCCUUUGUCAUCCUUCGCCU,
hsa-miR-2114:
(SEQ ID NO: 325)
UAGUCCCUUCCUUGAAGCGGUC,
hsa-miR-219-1-3p:
(SEQ ID NO: 326)
AGAGUUGAGUCUGGACGUCCCG,
hsa-miR-220c:
(SEQ ID NO: 327)
ACACAGGGCUGUUGUGAAGACU,
hsa-miR-23b*:
(SEQ ID NO: 328)
UGGGUUCCUGGCAUGCUGAUUU,
hsa-miR-27a*:
(SEQ ID NO: 329)
AGGGCUUAGCUGCUUGUGAGCA,
hsa-miR-299-3p:
(SEQ ID NO: 330)
UAUGUGGGAUGGUAAACCGCUU,
hsa-miR-299-5p:
(SEQ ID NO: 331)
UGGUUUACCGUCCCACAUACAU,
hsa-miR-300:
(SEQ ID NO: 332)
UAUACAAGGGCAGACUCUCUCU,
hsa-miR-30b*:
(SEQ ID NO: 333)
CUGGGAGGUGGAUGUUUACUUC,
hsa-miR-300-2*:
(SEQ ID NO: 334)
CUGGGAGAAGGCUGUUUACUCU,
hsa-miR-3116:
(SEQ ID NO: 335)
UGCCUGGAACAUAGUAGGGACU,
hsa-miR-3122:
(SEQ ID NO: 336)
GUUGGGACAAGAGGACGGUCUU,
hsa-miR-3124-3p:
(SEQ ID NO: 337)
ACUUUCCUCACUCCCGUGAAGU,
hsa-miR-3126-5p:
(SEQ ID NO: 338)
UGAGGGACAGAUGCCAGAAGCA,
hsa-miR-3133:
(SEQ ID NO: 339)
UAAAGAACUCUUAAAACCCAAU,
hsa-miR-3136-3p:
(SEQ ID NO: 340)
UGGCCCAACCUAUUCAGUUAGU,
hsa-miR-3140-3p:
(SEQ ID NO: 341)
AGCUUUUGGGAAUUCAGGUAGU,
hsa-miR-3156-5p:
(SEQ ID NO: 342)
AAAGAUCUGGAAGUGGGAGACA,
hsa-miR-3157-3p:
(SEQ ID NO: 343)
CUGCCCUAGUCUAGCUGAAGCU,
hsa-miR-3158-3p:
(SEQ ID NO: 344)
AAGGGCUUCCUCUCUGCAGGAC,
hsa-miR-3160-3p:
(SEQ ID NO: 345)
AGAGCUGAGACUAGAAAGCCCA,
hsa-miR-3163:
(SEQ ID NO: 346)
UAUAAAAUGAGGGCAGUAAGAC,
hsa-miR-3164:
(SEQ ID NO: 347)
UGUGACUUUAAGGGAAAUGGCG,
hsa-miR-3173-5p:
(SEQ ID NO: 348)
UGCCCUGCCUGUUUUCUCCUUU,
hsa-miR-3202:
(SEQ ID NO: 349)
UGGAAGGGAGAAGAGCUUUAAU,
hsa-miR-330-5p:
(SEQ ID NO: 350)
UCUCUGGGCCUGUGUCUUAGGC,
hsa-miR-33b*:
(SEQ ID NO: 351)
CAGUGCCUCGGCAGUGCAGCCC,
hsa-miR-345:
(SEQ ID NO: 352)
GCUGACUCCUAGUCCAGGGCUC,
hsa-miR-34a*:
(SEQ ID NO: 353)
CAAUCAGCAAGUAUACUGCCCU,
hsa-miR-3529-5p:
(SEQ ID NO: 354)
AGGUAGACUGGGAUUUGUUGUU,
hsa-miR-3617-5p:
(SEQ ID NO: 355)
AAAGACAUAGUUGCAAGAUGGG,
hsa-miR-3619-3p:
(SEQ ID NO: 356)
GGGACCAUCCUGCCUGCUGUGG,
hsa-miR-3622a-3p:
(SEQ ID NO: 357)
UCACCUGACCUCCCAUGCCUGU,
hsa-miR-3622a-5p:
(SEQ ID NO: 358)
CAGGCACGGGAGCUCAGGUGAG,
hsa-miR-363*:
(SEQ ID NO: 359)
CGGGUGGAUCACGAUGCAAUUU,
hsa-miR-3661:
(SEQ ID NO: 360)
UGACCUGGGACUCGGACAGCUG,
hsa-miR-3667-3p:
(SEQ ID NO: 361)
ACCUUCCUCUCCAUGGGUCUUU,
hsa-miR-3667-5p:
(SEQ ID NO: 362)
AAAGACCCAUUGAGGAGAAGGU,
hsa-miR-3677-3p:
(SEQ ID NO: 363)
CUCGUGGGCUCUGGCCACGGCC,
hsa-miR-3677-5p:
(SEQ ID NO: 364)
CAGUGGCCAGAGCCCUGCAGUG,
hsa-miR-3685:
(SEQ ID NO: 365)
UUUCCUACCCUACCUGAAGACU,
hsa-miR-3689a-3p:
(SEQ ID NO: 366)
CUGGGAGGUGUGAUAUCGUGGU,
hsa-miR-3689a-5p:
(SEQ ID NO: 367)
UGUGAUAUCAUGGUUCCUGGGA,
hsa-miR-3689b-3p:
(SEQ ID NO: 368)
CUGGGAGGUGUGAUAUUGUGGU,
hsa-miR-3689b-5p:
(SEQ ID NO: 369)
UGUGAUAUCAUGGUUCCUGGGA,
hsa-miR-3689c:
(SEQ ID NO: 370)
CUGGGAGGUGUGAUAUUGUGGU,
hsa-miR-3689d:
(SEQ ID NO: 371)
GGGAGGUGUGAUCUCACACUCG,
hsa-miR-3689e:
(SEQ ID NO: 372)
UGUGAUAUCAUGGUUCCUGGGA,
hsa-miR-3689f:
(SEQ ID NO: 373)
UGUGAUAUCGUGCUUCCUGGGA,
hsa-miR-377*:
(SEQ ID NO: 374)
AGAGGUUGCCCUUGGUGAAUUC,
hsa-miR-381:
(SEQ ID NO: 375)
UAUACAAGGGCAAGCUCUCUGU,
hsa-miR-3909:
(SEQ ID NO: 376)
UGUCCUCUAGGGCCUGCAGUCU,
hsa-miR-3913-3p:
(SEQ ID NO: 377)
AGACAUCAAGAUCAGUCCCAAA,
hsa-miR-3913-5p:
(SEQ ID NO: 378)
UUUGGGACUGAUCUUGAUGUCU,
hsa-miR-3923:
(SEQ ID NO: 379)
AACUAGUAAUGUUGGAUUAGGG,
hsa-miR-3934-3p:
(SEQ ID NO: 380)
UGCUCAGGUUGCACAGCUGGGA,
hsa-miR-422a:
(SEQ ID NO: 381)
ACUGGACUUAGGGUCAGAAGGC,
hsa-miR-4298:
(SEQ ID NO: 382)
CUGGGACAGGAGGAGGAGGCAG,
hsa-miR-431*:
(SEQ ID NO: 383)
CAGGUCGUCUUGCAGGGCUUCU,
hsa-miR-433:
(SEQ ID NO: 384)
AUCAUGAUGGGCUCCUCGGUGU,
hsa-miR-4423-5p:
(SEQ ID NO: 385)
AGUUGCCUUUUUGUUCCCAUGC,
hsa-miR-4425:
(SEQ ID NO: 386)
UGUUGGGAUUCAGCAGGACCAU,
hsa-miR-4428:
(SEQ ID NO: 387)
CAAGGAGACGGGAACAUGGAGC,
hsa-miR-4436b-3p:
(SEQ ID NO: 388)
CAGGGCAGGAAGAAGUGGACAA,
hsa-miR-4436b-5p:
(SEQ ID NO: 389)
GUCCACUUCUGCCUGCCCUGCC,
hsa-miR-4467:
(SEQ ID NO: 390)
UGGCGGCGGUAGUUAUGGGCUU,
hsa-miR-4471:
(SEQ ID NO: 391)
UGGGAACUUAGUAGAGGUUUAA,
hsa-miR-4475:
(SEQ ID NO: 392)
CAAGGGACCAAGCAUUCAUUAU,
hsa-miR-4476:
(SEQ ID NO: 393)
CAGGAAGGAUUUAGGGACAGGC,
hsa-miR-448:
(SEQ ID NO: 394)
UUGCAUAUGUAGGAUGUCCCAU,
hsa-miR-4490:
(SEQ ID NO: 395)
UCUGGUAAGAGAUUUGGGCAUA,
hsa-miR-4496:
(SEQ ID NO: 396)
GAGGAAACUGAAGCUGAGAGGG,
hsa-miR-450b-3p:
(SEQ ID NO: 397)
UUGGGAUCAUUUUGCAUCCAUA,
hsa-miR-4510:
(SEQ ID NO: 398)
UGAGGGAGUAGGAUGUAUGGUU,
hsa-miR-4511:
(SEQ ID NO: 399)
GAAGAACUGUUGCAUUUGCCCU,
hsa-miR-4513:
(SEQ ID NO: 400)
AGACUGACGGCUGGAGGCCCAU,
hsa-miR-4526:
(SEQ ID NO: 401)
GCUGACAGCAGGGCUGGCCGCU,
hsa-miR-4538:
(SEQ ID NO: 402)
GAGCUUGGAUGAGCUGGGCUGA,
hsa-miR-454*:
(SEQ ID NO: 403)
ACCCUAUCAAUAUUGUCUCUGC,
hsa-miR-4632-3p:
(SEQ ID NO: 404)
UGCCGCCCUCUCGCUGCUCUAG,
hsa-miR-4654:
(SEQ ID NO: 405)
UGUGGGAUCUGGAGGCAUCUGG,
hsa-miR-4669:
(SEQ ID NO: 406)
UGUGUCCGGGAAGUGGAGGAGG,
hsa-miR-4681:
(SEQ ID NO: 407)
AACGGGAAUGCAGGCUGUAUCU,
hsa-miR-4690-5p:
(SEQ ID NO: 408)
GAGCAGGCGAGGCUGGGCUGAA,
hsa-miR-4692:
(SEQ ID NO: 409)
UCAGGCAGUGUGGGUAUCAGAU,
hsa-miR-4695-5p:
(SEQ ID NO: 410)
CAGGAGGCAGUGGGCGAGCAGG,
hsa-miR-4717-5p:
(SEQ ID NO: 411)
UAGGCCACAGCCACCCAUGUGU,
hsa-miR-4721:
(SEQ ID NO: 412)
UGAGGGCUCCAGGUGACGGUGG,
hsa-miR-4722-3p:
(SEQ ID NO: 413)
ACCUGCCAGCACCUCCCUGCAG,
hsa-miR-4727-3p:
(SEQ ID NO: 414)
AUAGUGGGAAGCUGGCAGAUUC,
hsa-miR-4729:
(SEQ ID NO: 415)
UCAUUUAUCUGUUGGGAAGCUA,
hsa-miR-4733-3p:
(SEQ ID NO: 416)
CCACCAGGUCUAGCAUUGGGAU,
hsa-miR-4740-5p:
(SEQ ID NO: 417)
AGGACUGAUCCUCUCGGGCAGG,
hsa-miR-4747-5p:
(SEQ ID NO: 418)
AGGGAAGGAGGCUUGGUCUUAG,
hsa-miR-4750-5p:
(SEQ ID NO: 419)
CUCGGGCGGAGGUGGUUGAGUG,
hsa-miR-4755-3p:
(SEQ ID NO: 420)
AGCCAGGCUCUGAAGGGAAAGU,
hsa-miR-4755-5p:
(SEQ ID NO: 421)
UUUCCCUUCAGAGCCUGGCUUU,
hsa-miR-4764-3p:
(SEQ ID NO: 422)
UUAACUCCUUUCACACCCAUGG,
hsa-miR-4768-5p:
(SEQ ID NO: 423)
AUUCUCUCUGGAUCCCAUGGAU,
hsa-miR-4776-5p:
(SEQ ID NO: 424)
GUGGACCAGGAUGGCAAGGGCU,
hsa-miR-4779:
(SEQ ID NO: 425)
UAGGAGGGAAUAGUAAAAGCAG,
hsa-miR-4786-3p:
(SEQ ID NO: 426)
UGAAGCCAGCUCUGGUCUGGGC,
hsa-miR-4788:
(SEQ ID NO: 427)
UUACGGACCAGCUAAGGGAGGC,
hsa-miR-487a:
(SEQ ID NO: 428)
AAUCAUACAGGGACAUCCAGUU,
hsa-miR-487b:
(SEQ ID NO: 429)
AAUCGUACAGGGUCAUCCACUU,
hsa-miR-491-3p:
(SEQ ID NO: 430)
CUUAUGCAAGAUUCCCUUCUAC,
hsa-miR-494:
(SEQ ID NO: 431)
UGAAACAUACACGGGAAACCUC,
hsa-miR-500*:
(SEQ ID NO: 432)
AUGCACCUGGGCAAGGAUUCUG,
hsa-miR-5004-3p:
(SEQ ID NO: 433)
CUUGGAUUUUCCUGGGCCUCAG,
hsa-miR-5004-5p:
(SEQ ID NO: 434)
UGAGGACAGGGCAAAUUCACGA,
hsa-miR-502-3p:
(SEQ ID NO: 435)
AAUGCACCUGGGCAAGGAUUCA,
hsa-miR-504:
(SEQ ID NO: 436)
AGACCCUGGUCUGCACUCUAUC,
hsa-miR-505*:
(SEQ ID NO: 437)
GGGAGCCAGGAAGUAUUGAUGU,
hsa-miR-509-3p:
(SEQ ID NO: 438)
UGAUUGGUACGUCUGUGGGUAG,
hsa-miR-5100:
(SEQ ID NO: 439)
UUCAGAUCCCAGCGGUGCCUCU,
hsa-miR-5193:
(SEQ ID NO: 440)
UCCUCCUCUACCUCAUCCCAGU,
hsa-miR-5583-3p:
(SEQ ID NO: 441)
GAAUAUGGGUAUAUUAGUUUGG,
hsa-miR-5583-5p:
(SEQ ID NO: 442)
AAACUAAUAUACCCAUAUUCUG,
hsa-miR-5585-3p:
(SEQ ID NO: 443)
CUGAAUAGCUGGGACUACAGGU,
hsa-miR-5681a:
(SEQ ID NO: 444)
AGAAAGGGUGGCAAUACCUCUU,
hsa-miR-5681b:
(SEQ ID NO: 445)
AGGUAUUGCCACCCUUUCUAGU,
hsa-miR-5687:
(SEQ ID NO: 446)
UUAGAACGUUUUAGGGUCAAAU,
hsa-miR-5692a:
(SEQ ID NO: 447)
CAAAUAAUACCACAGUGGGUGU,
hsa-miR-5702:
(SEQ ID NO: 448)
UGAGUCAGCAACAUAUCCCAUG,
hsa-miR-5704:
(SEQ ID NO: 449)
UUAGGCCAUCAUCCCAUUAUGC,
hsa-miR-574-3p:
(SEQ ID NO: 450)
CACGCUCAUGCACACACCCACA,
hsa-miR-584:
(SEQ ID NO: 451)
UUAUGGUUUGCCUGGGACUGAG,
hsa-miR-616:
(SEQ ID NO: 452)
AGUCAUUGGAGGGUUUGAGCAG,
hsa-miR-616*:
(SEQ ID NO: 453)
ACUCAAAACCCUUCAGUGACUU,
hsa-miR-617:
(SEQ ID NO: 454)
AGACUUCCCAUUUGAAGGUGGC,
hsa-miR-630:
(SEQ ID NO: 455)
AGUAUUCUGUACCAGGGAAGGU,
hsa-miR-642:
(SEQ ID NO: 456)
GUCCCUCUCCAAAUGUGUCUUG,
hsa-miR-6499-3p:
(SEQ ID NO: 457)
AGCAGUGUUUGUUUUGCCCACA,
hsa-miR-6499-5p:
(SEQ ID NO: 458)
UCGGGCGCAAGAGCACUGCAGU,
hsa-miR-6501-5p:
(SEQ ID NO: 459)
AGUUGCCAGGGCUGCCUUUGGU,
hsa-miR-6507-3p:
(SEQ ID NO: 460)
CAAAGUCCUUCCUAUUUUUCCC,
hsa-miR-6508-3p:
(SEQ ID NO: 461)
UGGGCCAUGCAUUUCUAGAACU,
hsa-miR-6511a-3p:
(SEQ ID NO: 462)
CCUCACCAUCCCUUCUGCCUGC,
hsa-miR-6512-3p:
(SEQ ID NO: 463)
UUCCAGCCCUUCUAAUGGUAGG,
hsa-miR-654-5p:
(SEQ ID NO: 464)
UGGUGGGCCGCAGAACAUGUGC,
hsa-miR-660:
(SEQ ID NO: 465)
UACCCAUUGCAUAUCGGAGUUG,
hsa-miR-674:
(SEQ ID NO: 466)
GCACUGAGAUGGGAGUGGUGUA,
hsa-miR-7-2*:
(SEQ ID NO: 467)
CAACAAAUCCCAGUCUACCUAA,
hsa-miR-769-5p:
(SEQ ID NO: 468)
UGAGACCUCUGGGUUCUGAGCU,
hsa-miR-885-5p:
(SEQ ID NO: 469)
UCCAUUACACUACCCUGCCUCU,
hsa-miR-888*:
(SEQ ID NO: 470)
GACUGACACCUCUUUGGGUGAA,
hsa-miR-892b:
(SEQ ID NO: 471)
CACUGGCUCCUUUCUGGGUAGA,
hsa-miR-92a:
(SEQ ID NO: 472)
UAUUGCACUUGUCCCGGCCUGU,
hsa-miR-92b:
(SEQ ID NO: 473)
UAUUGCACUCGUCCCGGCCUCC,
hsa-miR-93*:
(SEQ ID NO: 474)
ACUGCUGAGCUAGCACUUCCCG,
hsa-miR-936:
(SEQ ID NO: 475)
ACAGUAGAGGGAGGAAUCGCAG,
hsa-miR-95:
(SEQ ID NO: 476)
UUCAACGGGUAUUUAUUGAGCA,
hsa-miR-99a:
(SEQ ID NO: 477)
AACCCGUAGAUCCGAUCUUGUG,
hsa-miR-99a*:
(SEQ ID NO: 478)
CAAGCUCGCUUCUAUGGGUCUG,
hsa-miR-99b:
(SEQ ID NO: 479)
CACCCGUAGAACCGACCUUGCG,
hsa-miR-99b*:
(SEQ ID NO: 480)
CAAGCUCGUGUCUGUGGGUCCG,
mmu-miR-291a-5p:
(SEQ ID NO: 481)
CAUCAAAGUGGAGGCCCUCUCU,
mmu-miR-291b-5p:
(SEQ ID NO: 482)
GAUCAAAGUGGAGGCCCUCUCC,
mmu-miR-294:
(SEQ ID NO: 483)
AAAGUGCUUCCCUUUUGUGUGU,
mmu-miR-350:
(SEQ ID NO: 484)
UUCACAAAGCCCAUACACUUUC,
mmu-miR-465a-3p:
(SEQ ID NO: 485)
GAUCAGGGCCUUUCUAAGUAGA,
mmu-miR-666-5p:
(SEQ ID NO: 486)
AGCGGGCACAGCUGUGAGAGCC,
mmu-miR-670:
(SEQ ID NO: 487)
AUCCCUGAGUGUAUGUGGUGAA,
mmu-miR-686:
(SEQ ID NO: 488)
AUUGCUUCCCAGACGGUGAAGA,
mmu-miR-695:
(SEQ ID NO: 489)
AGAUUGGGCAUAGGUGACUGAA,
mmu-miR-706:
(SEQ ID NO: 490)
AGAGAAACCCUGUCUCAAAAAA,
mmu-miR-742:
(SEQ ID NO: 491)
GAAAGCCACCAUGCUGGGUAAA,
mmu-miR-761:
(SEQ ID NO: 492)
GCAGCAGGGUGAAACUGACACA,
mmu-miR-763:
(SEQ ID NO: 493)
CCAGCUGGGAAGAACCAGUGGC,
mmu-miR-878-3p:
(SEQ ID NO: 494)
GCAUGACACCACACUGGGUAGA,
mmu-miR-883b-5p:
(SEQ ID NO: 495)
UACUGAGAAUGGGUAGCAGUCA,
rno-miR-349:
(SEQ ID NO: 496)
CAGCCCUGCUGUCUUAACCUCU,
hsa-let-70*:
(SEQ ID NO: 497)
UAGAGUUACACCCUGGGAGUUA,
hsa-miR-1226:
(SEQ ID NO: 498)
UCACCAGCCCUGUGUUCCCUAG,
hsa-miR-125b:
(SEQ ID NO: 499)
UCCCUGAGACCCUAACUUGUGA,
hsa-miR-125b-1*:
(SEQ ID NO: 500)
ACGGGUUAGGCUCUUGGGAGCU,
hsa-miR-1296:
(SEQ ID NO: 501)
UUAGGGCCCUGGCUCCAUCUCC,
hsa-miR-135b*:
(SEQ ID NO: 502)
AUGUAGGGCUAAAAGCCAUGGG,
hsa-miR-150:
(SEQ ID NO: 503)
UCUCCCAACCCUUGUACCAGUG,
hsa-miR-186*:
(SEQ ID NO: 504)
GCCCAAAGGUGAAUUUUUUGGG,
hsa-miR-187*:
(SEQ ID NO: 505)
GGCUACAACACAGGACCCGGGC,
hsa-miR-1915*:
(SEQ ID NO: 506)
ACCUUGCCUUGCUGCCCGGGCC,
hsa-miR-193a-5p:
(SEQ ID NO: 507)
UGGGUCUUUGCGGGCGAGAUGA,
hsa-miR-193b:
(SEQ ID NO: 508)
AACUGGCCCUCAAAGUCCCGCU,
hsa-miR-296-3p:
(SEQ ID NO: 509)
GAGGGUUGGGUGGAGGCUCUCC,
hsa-miR-300-1*:
(SEQ ID NO: 510)
CUGGGAGAGGGUUGUUUACUCC,
hsa-miR-320:
(SEQ ID NO: 511)
AAAAGCUGGGUUGAGAGGGCGA,
hsa-miR-328:
(SEQ ID NO: 512)
CUGGCCCUCUCUGCCCUUCCGU,
hsa-miR-331-5p:
(SEQ ID NO: 513)
CUAGGUAUGGUCCCAGGGAUCC,
hsa-miR-3646:
(SEQ ID NO: 514)
AAAAUGAAAUGAGCCCAGCCCA,
hsa-miR-3938:
(SEQ ID NO: 515)
AAUUCCCUUGUAGAUAACCCGG,
hsa-miR-425*:
(SEQ ID NO: 516)
AUCGGGAAUGUCGUGUCCGCCC,
hsa-miR-4446-3p:
(SEQ ID NO: 517)
CAGGGCUGGCAGUGACAUGGGU,
hsa-miR-4446-5p:
(SEQ ID NO: 518)
AUUUCCCUGCCAUUCCCUUGGC,
hsa-miR-4469:
(SEQ ID NO: 519)
GCUCCCUCUAGGGUCGCUCGGA,
hsa-miR-4482-5p:
(SEQ ID NO: 520)
AACCCAGUGGGCUAUGGAAAUG,
hsa-miR-4498:
(SEQ ID NO: 521)
UGGGCUGGCAGGGCAAGUGCUG,
hsa-miR-4512:
(SEQ ID NO: 522)
CAGGGCCUCACUGUAUCGCCCA,
hsa-miR-4515:
(SEQ ID NO: 523)
AGGACUGGACUCCCGGCAGCCC,
hsa-miR-4539:
(SEQ ID NO: 524)
GCUGAACUGGGCUGAGCUGGGC,
hsa-miR-4646-5p:
(SEQ ID NO: 525)
ACUGGGAAGAGGAGCUGAGGGA,
hsa-miR-4685-3p:
(SEQ ID NO: 526)
UCUCCCUUCCUGCCCUGGCUAG,
hsa-miR-4713-3p:
(SEQ ID NO: 527)
UGGGAUCCAGACAGUGGGAGAA,
hsa-miR-4713-5p:
(SEQ ID NO: 528)
UUCUCCCACUACCAGGCUCCCA,
hsa-miR-4726-3p:
(SEQ ID NO: 529)
ACCCAGGUUCCCUCUGGCCGCA,
hsa-miR-4733-5p:
(SEQ ID NO: 530)
AAUCCCAAUGCUAGACCCGGUG,
hsa-miR-4734:
(SEQ ID NO: 531)
GCUGCGGGCUGCGGUCAGGGCG,
hsa-miR-4740-3p:
(SEQ ID NO: 532)
GCCCGAGAGGAUCCGUCCCUGC,
hsa-miR-4780:
(SEQ ID NO: 533)
ACCCUUGAGCCUGAUCCCUAGC,
hsa-miR-483-5p:
(SEQ ID NO: 534)
AAGACGGGAGGAAAGAAGGGAG,
hsa-miR-501-3p:
(SEQ ID NO: 535)
AAUGCACCCGGGCAAGGAUUCU,
hsa-miR-501-5p:
(SEQ ID NO: 536)
AAUCCUUUGUCCCUGGGUGAGA,
hsa-miR-5187-5p:
(SEQ ID NO: 537)
UGGGAUGAGGGAUUGAAGUGGA,
hsa-miR-5584-3p:
(SEQ ID NO: 538)
UAGUUCUUCCCUUUGCCCAAUU,
hsa-miR-5584-5p:
(SEQ ID NO: 539)
CAGGGAAAUGGGAAGAACUAGA,
hsa-miR-5685:
(SEQ ID NO: 540)
ACAGCCCAGCAGUUAUCACGGG,
hsa-miR-615-3p:
(SEQ ID NO: 541)
UCCGAGCCUGGGUCUCCCUCUU,
hsa-miR-629*:
(SEQ ID NO: 542)
GUUCUCCCAACGUAAGCCCAGC,
hsa-miR-877*:
(SEQ ID NO: 543)
UCCUCUUCUCCCUCCUCCCAGG,
hsa-miR-887:
(SEQ ID NO: 544)
GUGAACGGGCGCCAUCCCGAGG,
hsa-miR-92b*:
(SEQ ID NO: 545)
AGGGACGGGACGCGGUGCAGUG,
hsa-miR-933:
(SEQ ID NO: 546)
UGUGCGCAGGGAGACCUCUCCC,
hsa-miR-938:
(SEQ ID NO: 547)
UGCCCUUAAAGGUGAACCCAGU,
hsa-miR-1914:
(SEQ ID NO: 548)
CCCUGUGCCCGGCCCACUUCUG,
hsa-miR-3620-3p:
(SEQ ID NO: 549)
UCACCCUGCAUCCCGCACCCAG,
hsa-miR-3940-3p:
(SEQ ID NO: 550)
CAGCCCGGAUCCCAGCCCACUU,
hsa-miR-4687-5p:
(SEQ ID NO: 551)
CAGCCCUCCUCCCGCACCCAAA,
hsa-miR-4747-3p:
(SEQ ID NO: 552)
AAGGCCCGGGCUUUCCUCCCAG,
hsa-miR-874:
(SEQ ID NO: 553)
CUGCCCUGGCCCGAGGGACCGA,
hsa-miR-3620-5p:
(SEQ ID NO: 554)
GUGGGCUGGGCUGGGCUGGGCC,
hsa-miR-103:
(SEQ ID NO: 555)
AGCAGCAUUGUACAGGGCUAUGA,
hsa-miR-107:
(SEQ ID NO: 556)
AGCAGCAUUGUACAGGGCUAUCA,
hsa-miR-10a:
(SEQ ID NO: 557)
UACCCUGUAGAUCCGAAUUUGUG,
hsa-miR-10b:
(SEQ ID NO: 558)
UACCCUGUAGAACCGAAUUUGUG,
hsa-miR-149:
(SEQ ID NO: 559)
UCUGGCUCCGUGUCUUCACUCCC,
hsa-miR-181b:
(SEQ ID NO: 560)
AACAUUCAUUGCUGUCGGUGGGU,
hsa-miR-181d:
(SEQ ID NO: 561)
AACAUUCAUUGUUGUCGGUGGGU,
hsa-miR-18a*:
(SEQ ID NO: 562)
ACUGCCCUAAGUGCUCCUUCUGG,
hsa-miR-191:
(SEQ ID NO: 563)
CAACGGAAUCCCAAAAGCAGCUG,
hsa-miR-1911:
(SEQ ID NO: 564)
UGAGUACCGCCAUGUCUGUUGGG,
hsa-miR-199a-5p:
(SEQ ID NO: 565)
CCCAGUGUUCAGACUACCUGUUC,
hsa-miR-199b-5p:
(SEQ ID NO: 566)
CCCAGUGUUUAGACUAUCUGUUC,
hsa-miR-200c:
(SEQ ID NO: 567)
UAAUACUGCCGGGUAAUGAUGGA,
hsa-miR-221:
(SEQ ID NO: 568)
AGCUACAUUGUCUGCUGGGUUUC,
hsa-miR-3131:
(SEQ ID NO: 569)
UCGAGGACUGGUGGAAGGGCCUU,
hsa-miR-3136-5p:
(SEQ ID NO: 570)
CUGACUGAAUAGGUAGGGUCAUU,
hsa-miR-3161:
(SEQ ID NO: 571)
CUGAUAAGAACAGAGGCCCAGAU,
hsa-miR-3187-5p:
(SEQ ID NO: 572)
CCUGGGCAGCGUGUGGCUGAAGG,
hsa-miR-3192:
(SEQ ID NO: 573)
UCUGGGAGGUUGUAGCAGUGGAA,
hsa-miR-3199:
(SEQ ID NO: 574)
AGGGACUGCCUUAGGAGAAAGUU,
hsa-miR-339-5p:
(SEQ ID NO: 575)
UCCCUGUCCUCCAGGAGCUCACG,
hsa-miR-342-3p:
(SEQ ID NO: 576)
UCUCACACAGAAAUCGCACCCGU,
hsa-miR-346:
(SEQ ID NO: 577)
UGUCUGCCCGCAUGCCUGCCUCU,
hsa-miR-3614-5p:
(SEQ ID NO: 578)
CCACUUGGAUCUGAAGGCUGCCC,
hsa-miR-3616-3p:
(SEQ ID NO: 579)
CGAGGGCAUUUCAUGAUGCAGGC,
hsa-miR-3617-3p:
(SEQ ID NO: 580)
CAUCAGCACCCUAUGUCCUUUCU,
hsa-miR-3663-3p:
(SEQ ID NO: 581)
UGAGCACCACACAGGCCGGGCGC,
hsa-miR-3690:
(SEQ ID NO: 582)
ACCUGGACCCAGCGUAGACAAAG,
hsa-miR-3922-5p:
(SEQ ID NO: 583)
UCAAGGCCAGAGGUCCCACAGCA,
hsa-miR-3944-3p:
(SEQ ID NO: 584)
UUCGGGCUGGCCUGCUGCUCCGG,
hsa-miR-409-5p:
(SEQ ID NO: 585)
AGGUUACCCGAGCAACUUUGCAU,
hsa-miR-421:
(SEQ ID NO: 586)
AUCAACAGACAUUAAUUGGGCGC,
hsa-miR-425:
(SEQ ID NO: 587)
AAUGACACGAUCACUCCCGUUGA,
hsa-miR-432:
(SEQ ID NO: 588)
UCUUGGAGUAGGUCAUUGGGUGG,
hsa-miR-4461:
(SEQ ID NO: 589)
GAUUGAGACUAGUAGGGCUAGGC,
hsa-miR-454:
(SEQ ID NO: 590)
UAGUGCAAUAUUGCUUAUAGGGU,
hsa-miR-4653-3p:
(SEQ ID NO: 591)
UGGAGUUAAGGGUUGCUUGGAGA,
hsa-miR-4666b:
(SEQ ID NO: 592)
UUGCAUGUCAGAUUGUAAUUCCC,
hsa-miR-4668-5p:
(SEQ ID NO: 593)
AGGGAAAAAAAAAAGGAUUUGUC,
hsa-miR-4683:
(SEQ ID NO: 594)
UGGAGAUCCAGUGCUCGCCCGAU,
hsa-miR-4686:
(SEQ ID NO: 595)
UAUCUGCUGGGCUUUCUGGUGUU,
hsa-miR-4722-5p:
(SEQ ID NO: 596)
GGCAGGAGGGCUGUGCCAGGUUG,
hsa-miR-4726-5p:
(SEQ ID NO: 597)
AGGGCCAGAGGAGCCUGGAGUGG,
hsa-miR-4730:
(SEQ ID NO: 598)
CUGGCGGAGCCCAUUCCAUGCCA,
hsa-miR-4732-5p:
(SEQ ID NO: 599)
UGUAGAGCAGGGAGCAGGAAGCU,
hsa-miR-4742-5p:
(SEQ ID NO: 600)
UCAGGCAAAGGGAUAUUUACAGA,
hsa-miR-4743-5p:
(SEQ ID NO: 601)
UGGCCGGAUGGGACAGGAGGCAU,
hsa-miR-4745-3p:
(SEQ ID NO: 602)
UGGCCCGGCGACGUCUCACGGUC,
hsa-miR-4746-5p:
(SEQ ID NO: 603)
CCGGUCCCAGGAGAACCUGCAGA,
hsa-miR-4754:
(SEQ ID NO: 604)
AUGCGGACCUGGGUUAGCGGAGU,
hsa-miR-4756-5p:
(SEQ ID NO: 605)
CAGGGAGGCGCUCACUCUCUGCU,
hsa-miR-4767:
(SEQ ID NO: 606)
CGCGGGCGCUCCUGGCCGCCGCC,
hsa-miR-492:
(SEQ ID NO: 607)
AGGACCUGCGGGACAAGAUUCUU,
hsa-miR-500:
(SEQ ID NO: 608)
UAAUCCUUGCUACCUGGGUGAGA,
hsa-miR-5003-5p:
(SEQ ID NO: 609)
UCACAACAACCUUGCAGGGUAGA,
hsa-miR-503:
(SEQ ID NO: 610)
UAGCAGCGGGAACAGUUCUGCAG,
hsa-miR-508-5p:
(SEQ ID NO: 611)
UACUCCAGAGGGCGUCACUCAUG,
hsa-miR-5087:
(SEQ ID NO: 612)
GGGUUUGUAGCUUUGCUGGCAUG,
hsa-miR-5089-3p:
(SEQ ID NO: 613)
AUGCUACUCGGAAAUCCCACUGA,
hsa-miR-5188:
(SEQ ID NO: 614)
AAUCGGACCCAUUUAAACCGGAG,
hsa-miR-5589-3p:
(SEQ ID NO: 615)
UGCACAUGGCAACCUAGCUCCCA,
hsa-miR-605:
(SEQ ID NO: 616)
UAAAUCCCAUGGUGCCUUCUCCU,
hsa-miR-635:
(SEQ ID NO: 617)
ACUUGGGCACUGAAACAAUGUCC,
hsa-miR-6506-5p:
(SEQ ID NO: 618)
ACUGGGAUGUCACUGAAUAUGGU,
hsa-miR-6513-5p:
(SEQ ID NO: 619)
UUUGGGAUUGACGCCACAUGUCU,
hsa-miR-657:
(SEQ ID NO: 620)
GGCAGGUUCUCACCCUCUCUAGG,
hsa-miR-668:
(SEQ ID NO: 621)
UGUCACUCGGCUCGGCCCACUAC,
hsa-miR-708:
(SEQ ID NO: 622)
AAGGAGCUUACAAUCUAGCUGGG,
hsa-miR-770-5p:
(SEQ ID NO: 623)
UCCAGUACCACGUGUCAGGGCCA,
hsa-miR-886-5p:
(SEQ ID NO: 624)
CGGGUCGGAGUUAGCUCAAGCGG,
hsa-miR-92a-1*:
(SEQ ID NO: 625)
AGGUUGGGAUCGGUUGCAAUGCU,
hsa-miR-941:
(SEQ ID NO: 626)
CACCCGGCUGUGUGCACAUGUGC,
mmu-miR-207:
(SEQ ID NO: 627)
GCUUCUCCUGGCUCUCCUCCCUC,
hsa-miR-1229:
(SEQ ID NO: 628)
CUCUCACCACUGCCCUCCCACAG,
hsa-miR-1266:
(SEQ ID NO: 629)
CCUCAGGGCUGUAGAACAGGGCU,
hsa-miR-145:
(SEQ ID NO: 630)
GUCCAGUUUUCCCAGGAAUCCCU,
hsa-miR-1538:
(SEQ ID NO: 631)
CGGCCCGGGCUGCUGCUGUUCCU,
hsa-miR-3127-5p:
(SEQ ID NO: 632)
AUCAGGGCUUGUGGAAUGGGAAG,
hsa-miR-3680-3p:
(SEQ ID NO: 633)
UUUUGCAUGACCCUGGGAGUAGG,
hsa-miR-3945:
(SEQ ID NO: 634)
AGGGCAUAGGAGAGGGUUGAUAU,
hsa-miR-4462:
(SEQ ID NO: 635)
UGACACGGAGGGUGGCUUGGGAA,
hsa-miR-4632-5p:
(SEQ ID NO: 636)
GAGGGCAGCGUGGGUGUGGCGGA,
hsa-miR-4656:
(SEQ ID NO: 637)
UGGGCUGAGGGCAGGAGGCCUGU,
hsa-miR-5088:
(SEQ ID NO: 638)
CAGGGCUCAGGGAUUGGAUGGAG,
hsa-miR-602:
(SEQ ID NO: 639)
GACACGGGCGACAGCUGCGGCCC,
hsa-miR-636:
(SEQ ID NO: 640)
UGUGCUUGCUCGUCCCGCCCGCA,
hsa-miR-675:
(SEQ ID NO: 641)
UGGUGCGGAGAGGGCCCACAGUG,
mmu-miR-667:
(SEQ ID NO: 642)
UGACACCUGCCACCCAGCCCAAG,
hsa-miR-1182:
(SEQ ID NO: 643)
GAGGGUCUUGGGAGGGAUGUGAC,
hsa-miR-4640-5p:
(SEQ ID NO: 644)
UGGGCCAGGGAGCAGCUGGUGGG,
hsa-miR-1291:
(SEQ ID NO: 645)
UGGCCCUGACUGAAGACCAGCAGU,
hsa-miR-1301:
(SEQ ID NO: 646)
UUGCAGCUGCCUGGGAGUGACUUC,
hsa-miR-2277-5p:
(SEQ ID NO: 647)
AGCGCGGGCUGAGCGCUGCCAGUC,
hsa-miR-3132:
(SEQ ID NO: 648)
UGGGUAGAGAAGGAGCUCAGAGGA,
hsa-miR-3138:
(SEQ ID NO: 649)
UGUGGACAGUGAGGUAGAGGGAGU,
hsa-miR-3651:
(SEQ ID NO: 650)
CAUAGCCCGGUCGCUGGUACAUGA,
hsa-miR-3687:
(SEQ ID NO: 651)
CCCGGACAGGCGUUCGUGCGACGU,
hsa-miR-3978:
(SEQ ID NO: 652)
GUGGAAAGCAUGCAUCCAGGGUGU,
hsa-miR-4641:
(SEQ ID NO: 653)
UGCCCAUGCCAUACUUUUGCCUCA,
hsa-miR-4751:
(SEQ ID NO: 654)
AGAGGACCCGUAGCUGCUAGAAGG,
hsa-miR-4769-5p:
(SEQ ID NO: 655)
GGUGGGAUGGAGAGAAGGUAUGAG,
hsa-miR-4793-5p:
(SEQ ID NO: 656)
ACAUCCUGCUCCACAGGGCAGAGG,
hsa-miR-5001-5p:
(SEQ ID NO: 657)
AGGGCUGGACUCAGCGGCGGAGCU,
hsa-miR-5189:
(SEQ ID NO: 658)
UCUGGGCACAGGCGGAUGGACAGG,
hsa-miR-589*:
(SEQ ID NO: 659)
UCAGAACAAAUGCCGGUUCCCAGA,
hsa-miR-619:
(SEQ ID NO: 660)
GACCUGGACAUGUUUGUGCCCAGU,
hsa-miR-661:
(SEQ ID NO: 661)
UGCCUGGGUCUCUGGCCUGCGCGU,
mmu-miR-290-3p:
(SEQ ID NO: 662)
AAAGUGCCGCCUAGUUUUAAGCCC,
hsa-miR-125a-5p:
(SEQ ID NO: 663)
UCCCUGAGACCCUUUAACCUGUGA,
hsa-miR-298:
(SEQ ID NO: 664)
AGCAGAAGCAGGGAGGUUCUCCCA,
hsa-miR-3147:
(SEQ ID NO: 665)
GGUUGGGCAGUGAGGAGGGUGUGA,
hsa-miR-3613-3p:
(SEQ ID NO: 666)
ACAAAAAAAAAAGCCCAACCCUUC,
mmu-miR-351:
(SEQ ID NO: 667)
UCCCUGAGGAGCCCUUUGAGCCUG,
hsa-miR-1273:
(SEQ ID NO: 668)
GGGCGACAAAGCAAGACUCUUUCUU,
hsa-miR-658:
(SEQ ID NO: 669)
GGCGGAGGGAAGUAGGUCCGUUGGU,
hsa-miR-921:
(SEQ ID NO: 670)
CUAGUGAGGGACAGAACCAGGAUUC,
hsa-miR-1292:
(SEQ ID NO: 671)
UGGGAACGGGUUCCGGCAGACGCUG,
hsa-miR-612:
(SEQ ID NO: 672)
GCUGGGCAGGGCUUCUGAGCUCCUU,
hsa-miR-638:
(SEQ ID NO: 673)
AGGGAUCGCGGGCGGGUGGCGGCCU,
hsa-miR-4518:
(SEQ ID NO: 674)
GCUCAGGGAUGAUAACUGUGCUGAGA,
hsa-miR-1183:
(SEQ ID NO: 675)
CACUGUAGGUGAUGGUGAGAGUGGGCA,
hsa-miR-3178:
(SEQ ID NO: 676)
GGGGCGCGGCCGGAUCG,
hsa-miR-4258:
(SEQ ID NO: 677)
CCCCGCCACCGCCUUGG,
hsa-miR-4283:
(SEQ ID NO: 678)
UGGGGCUCAGCGAGUUU,
hsa-miR-4286:
(SEQ ID NO: 679)
ACCCCACUCCUGGUACC,
hsa-miR-4483:
(SEQ ID NO: 680)
GGGGUGGUCUGUUGUUG,
hsa-miR-4534:
(SEQ ID NO: 681)
GGAUGGAGGAGGGGUCU,
hsa-miR-4463:
(SEQ ID NO: 682)
GAGACUGGGGUGGGGCC,
hsa-miR-4492:
(SEQ ID NO: 683)
GGGGCUGGGCGCGCGCC,
hsa-miR-4508:
(SEQ ID NO: 684)
GCGGGGCUGGGCGCGCG,
hsa-miR-4516:
(SEQ ID NO: 685)
GGGAGAAGGGUCGGGGC,
hsa-miR-4532:
(SEQ ID NO: 686)
CCCCGGGGAGCCCGGCG,
hsa-miR-3665:
(SEQ ID NO: 687)
AGCAGGUGCGGGGCGGCG,
hsa-miR-4257:
(SEQ ID NO: 688)
CCAGAGGUGGGGACUGAG,
hsa-miR-4323:
(SEQ ID NO: 689)
CAGCCCCACAGCCUCAGA,
hsa-miR-4514:
(SEQ ID NO: 690)
ACAGGCAGGAUUGGGGAA,
mmu-miR-720:
(SEQ ID NO: 691)
AUCUCGCUGGGGCCUCCA,
hsa-miR-3196:
(SEQ ID NO: 692)
CGGGGCGGCAGGGGCCUC,
hsa-miR-4284:
(SEQ ID NO: 693)
GGGCUCACAUCACCCCAU,
hsa-miR-4292:
(SEQ ID NO: 694)
CCCCUGGGCCGGCCUUGG,
hsa-miR-4466:
(SEQ ID NO: 695)
GGGUGCGGGCCGGCGGGG,
hsa-miR-3180:
(SEQ ID NO: 696)
UGGGGCGGAGCUUCCGGAG,
hsa-miR-4312:
(SEQ ID NO: 697)
GGCCUUGUUCCUGUCCCCA,
hsa-miR-593:
(SEQ ID NO: 698)
UGUCUCUGCUGGGGUUUCU,
hsa-miR-6165:
(SEQ ID NO: 699)
CAGCAGGAGGUGAGGGGAG,
mmu-miR-327:
(SEQ ID NO: 700)
ACUUGAGGGGCAUGAGGAU,
hsa-miR-2861:
(SEQ ID NO: 701)
GGGGCCUGGCGGUGGGCGG,
hsa-miR-4260:
(SEQ ID NO: 702)
CUUGGGGCAUGGAGUCCCA,
hsa-miR-4634:
(SEQ ID NO: 703)
CGGCGCGACCGGCCCGGGG,
hsa-miR-6132:
(SEQ ID NO: 704)
AGCAGGGCUGGGGAUUGCA,
hsa-miR-6090:
(SEQ ID NO: 705)
GGGGAGCGAGGGGCGGGGC,
hsa-miR-1238:
(SEQ ID NO: 706)
CUUCCUCGUCUGUCUGCCCC,
hsa-miR-3187-3p:
(SEQ ID NO: 707)
UUGGCCAUGGGGCUGCGCGG,
hsa-miR-3190-5p:
(SEQ ID NO: 708)
UCUGGCCAGCUACGUCCCCA,
hsa-miR-324-3p:
(SEQ ID NO: 709)
ACUGCCCCAGGUGCUGCUGG,
hsa-miR-3713:
(SEQ ID NO: 710)
GGUAUCCGUUUGGGGAUGGU,
hsa-miR-4448:
(SEQ ID NO: 711)
GGCUCCUUGGUCUAGGGGUA,
hsa-miR-5739:
(SEQ ID NO: 712)
GCGGAGAGAGAAUGGGGAGC,
hsa-miR-665:
(SEQ ID NO: 713)
ACCAGGAGGCUGAGGCCCCU,
hsa-miR-920:
(SEQ ID NO: 714)
GGGGAGCUGUGGAAGCAGUA,
hsa-miR-1227:
(SEQ ID NO: 715)
CGUGCCACCCUUUUCCCCAG,
hsa-miR-3621:
(SEQ ID NO: 716)
CGCGGGUCGGGGUCUGCAGG,
hsa-miR-4484:
(SEQ ID NO: 717)
AAAAGGCGGGAGAAGCCCCA,
hsa-miR-760:
(SEQ ID NO: 718)
CGGCUCUGGGUCUGUGGGGA,
hsa-miR-1915:
(SEQ ID NO: 719)
CCCCAGGGCGACGCGGCGGG,
hsa-miR-3940-5p:
(SEQ ID NO: 720)
GUGGGUUGGGGCGGGCUCUG,
hsa-miR-4270:
(SEQ ID NO: 721)
UCAGGGAGUCAGGGGAGGGC,
hsa-miR-4651:
(SEQ ID NO: 722)
CGGGGUGGGUGAGGUCGGGC,
hsa-miR-5787:
(SEQ ID NO: 723)
GGGCUGGGGCGCGGGGAGGU,
mmu-miR-705:
(SEQ ID NO: 724)
GGUGGGAGGUGGGGUGGGCA,
hsa-miR-1224-3p:
(SEQ ID NO: 725)
CCCCACCUCCUCUCUCCUCAG,
hsa-miR-1267:
(SEQ ID NO: 726)
CCUGUUGAAGUGUAAUCCCCA,
hsa-miR-1908:
(SEQ ID NO: 727)
CGGCGGGGACGGCGAUUGGUC,
hsa-miR-2355-5p:
(SEQ ID NO: 728)
AUCCCCAGAUACAAUGGACAA,
hsa-miR-3177-3p:
(SEQ ID NO: 729)
UGCACGGCACUGGGGACACGU,
hsa-miR-342-5p:
(SEQ ID NO: 730)
AGGGGUGCUAUCUGUGAUUGA,
hsa-miR-4489:
(SEQ ID NO: 731)
UGGGGCUAGUGAUGCAGGACG,
hsa-miR-4649-3p:
(SEQ ID NO: 732)
UCUGAGGCCUGCCUCUCCCCA,
hsa-miR-4748:
(SEQ ID NO: 733)
GAGGUUUGGGGAGGAUUUGCU,
hsa-miR-4781-5p:
(SEQ ID NO: 734)
UAGCGGGGAUUCCAAUAUUGG,
hsa-miR-486-3p:
(SEQ ID NO: 735)
CGGGGCAGCUCAGUACAGGAU,
hsa-miR-5003-3p:
(SEQ ID NO: 736)
UACUUUUCUAGGUUGUUGGGG,
hsa-miR-3151:
(SEQ ID NO: 737)
GGUGGGGCAAUGGGAUCAGGU,
hsa-miR-331-3p:
(SEQ ID NO: 738)
GCCCCUGGGCCUAUCCUAGAA,
hsa-miR-3648:
(SEQ ID NO: 739)
AGCCGCGGGGAUCGCCGAGGG,
hsa-miR-4322:
(SEQ ID NO: 740)
CUGUGGGCUCAGCGCGUGGGG,
hsa-miR-4667-3p:
(SEQ ID NO: 741)
UCCCUCCUUCUGUCCCCACAG,
hsa-miR-5572:
(SEQ ID NO: 742)
GUUGGGGUGCAGGGGUCUGCU,
hsa-miR-5587-3p:
(SEQ ID NO: 743)
GCCCCGGGCAGUGUGAUCAUC,
mmu-miR-689:
(SEQ ID NO: 744)
CGUCCCCGCUCGGCGGGGUCC,
hsa-miR-1207-5p:
(SEQ ID NO: 745)
UGGCAGGGAGGCUGGGAGGGG,
hsa-miR-1470:
(SEQ ID NO: 746)
GCCCUCCGCCCGUGCACCCCG,
hsa-miR-3162-3p:
(SEQ ID NO: 747)
UCCCUACCCCUCCACUCCCCA,
hsa-miR-3189-3p:
(SEQ ID NO: 748)
CCCUUGGGUCUGAUGGGGUAG,
hsa-miR-4655-3p:
(SEQ ID NO: 749)
ACCCUCGUCAGGUCCCCGGGG,
mmu-miR-702:
(SEQ ID NO: 750)
UGCCCACCCUUUACCCCGCUC,
hsa-miR-10a*:
(SEQ ID NO: 751)
CAAAUUCGUAUCUAGGGGAAUA,
hsa-miR-10b*:
(SEQ ID NO: 752)
ACAGAUUCGAUUCUAGGGGAAU,
hsa-miR-1236:
(SEQ ID NO: 753)
CCUCUUCCCCUUGUCUCUCCAG,
hsa-miR-1303:
(SEQ ID NO: 754)
UUUAGAGACGGGGUCUUGCUCU,
hsa-miR-1323:
(SEQ ID NO: 755)
UCAAAACUGAGGGGCAUUUUCU,
hsa-miR-133a:
(SEQ ID NO: 756)
UUUGGUCCCCUUCAACCAGCUG,
hsa-miR-133b:
(SEQ ID NO: 757)
UUUGGUCCCCUUCAACCAGCUA,
hsa-miR-134:
(SEQ ID NO: 758)
UGUGACUGGUUGACCAGAGGGG,
hsa-miR-185*:
(SEQ ID NO: 759)
AGGGGCUGGCUUUCCUCUGGUC,
hsa-miR-191*:
(SEQ ID NO: 760)
GCUGCGCUUGGAUUUCGUCCCC,
hsa-miR-194*:
(SEQ ID NO: 761)
CCAGUGGGGCUGCUGUUAUCUG,
hsa-miR-198:
(SEQ ID NO: 762)
GGUCCAGAGGGGAGAUAGGUUC,
hsa-miR-2110:
(SEQ ID NO: 763)
UUGGGGAAACGGCCGCUGAGUG,
hsa-miR-223:
(SEQ ID NO: 764)
UGUCAGUUUGUCAAAUACCCCA,
hsa-miR-30d:
(SEQ ID NO: 765)
UGUAAACAUCCCCGACUGGAAG,
hsa-miR-3127-3p:
(SEQ ID NO: 766)
UCCCCUUCUGCAGGCCUGCUGG,
hsa-miR-3144-5p:
(SEQ ID NO: 767)
AGGGGACCAAAGAGAUAUAUAG,
hsa-miR-3150a-3p:
(SEQ ID NO: 768)
CUGGGGAGAUCCUCGAGGUUGG,
hsa-miR-3150b-5p:
(SEQ ID NO: 769)
CAACCUCGAGGAUCUCCCCAGC,
hsa-miR-3152-3p:
(SEQ ID NO: 770)
UGUGUUAGAAUAGGGGCAAUAA,
hsa-miR-3170:
(SEQ ID NO: 771)
CUGGGGUUCUGAGACAGACAGU,
hsa-miR-3175:
(SEQ ID NO: 772)
CGGGGAGAGAACGCAGUGACGU,
hsa-miR-3179:
(SEQ ID NO: 773)
AGAAGGGGUGAAAUUUAAACGU,
hsa-miR-3180-3p:
(SEQ ID NO: 774)
UGGGGCGGAGCUUCCGGAGGCC,
hsa-miR-3198:
(SEQ ID NO: 775)
GUGGAGUCCUGGGGAAUGGAGA,
hsa-miR-361-5p:
(SEQ ID NO: 776)
UUAUCAGAAUCUCCAGGGGUAC,
hsa-miR-365:
(SEQ ID NO: 777)
UAAUGCCCCUAAAAAUCCUUAU,
hsa-miR-370:
(SEQ ID NO: 778)
GCCUGCUGGGGUGGAACCUGGU,
hsa-miR-3714:
(SEQ ID NO: 779)
GAAGGCAGCAGUGCUCCCCUGU,
hsa-miR-3936:
(SEQ ID NO: 780)
UAAGGGGUGUAUGGCAGAUGCA,
hsa-miR-409-3p:
(SEQ ID NO: 781)
GAAUGUUGCUCGGUGAACCCCU,
hsa-miR-4440:
(SEQ ID NO: 782)
UGUCGUGGGGCUUGCUGGCUUG,
hsa-miR-4450:
(SEQ ID NO: 783)
UGGGGAUUUGGAGAAGUGGUGA,
hsa-miR-4465:
(SEQ ID NO: 784)
CUCAAGUAGUCUGACCAGGGGA,
hsa-miR-4482-3p:
(SEQ ID NO: 785)
UUUCUAUUUCUCAGUGGGGCUC,
hsa-miR-4642:
(SEQ ID NO: 786)
AUGGCAUCGUCCCCUGGUGGCU,
hsa-miR-4652-5p:
(SEQ ID NO: 787)
AGGGGACUGGUUAAUAGAACUA,
hsa-miR-4664-3p:
(SEQ ID NO: 788)
CUUCCGGUCUGUGAGCCCCGUC,
hsa-miR-4667-5p:
(SEQ ID NO: 789)
ACUGGGGAGCAGAAGGAGAACC,
hsa-miR-4675:
(SEQ ID NO: 790)
GGGGCUGUGAUUGACCAGCAGG,
hsa-miR-4700-5p:
(SEQ ID NO: 791)
UCUGGGGAUGAGGACAGUGUGU,
hsa-miR-4701-5p:
(SEQ ID NO: 792)
UUGGCCACCACACCUACCCCUU,
hsa-miR-4714-5p:
(SEQ ID NO: 793)
AACUCUGACCCCUUAGGUUGAU,
hsa-miR-486-5p:
(SEQ ID NO: 794)
UCCUGUACUGAGCUGCCCCGAG,
hsa-miR-5195-5p:
(SEQ ID NO: 795)
AACCCCUAAGGCAACUGGAUGG,
hsa-miR-634:
(SEQ ID NO: 796)
AACCAGCACCCCAACUUUGGAC,
hsa-miR-6505-5p:
(SEQ ID NO: 797)
UUGGAAUAGGGGAUAUCUCAGC,
hsa-miR-6510-5p:
(SEQ ID NO: 798)
CAGCAGGGGAGAGAGAGGAGUC,
hsa-miR-6717-5p:
(SEQ ID NO: 799)
AGGCGAUGUGGGGAUGUAGAGA,
hsa-miR-6723-5p:
(SEQ ID NO: 800)
AUAGUCCGAGUAACGUCGGGGC,
hsa-miR-766:
(SEQ ID NO: 801)
ACUCCAGCCCCACAGCCUCAGC,
hsa-miR-885-3p:
(SEQ ID NO: 802)
AGGCAGCGGGGUGUAGUGGAUA,
mmu-miR-710:
(SEQ ID NO: 803)
CCAAGUCUUGGGGAGAGUUGAG,
rno-miR-664:
(SEQ ID NO: 804)
UAUUCAUUUACUCCCCAGCCUA,
hsa-miR-1234:
(SEQ ID NO: 805)
UCGGCCUGACCACCCACCCCAC,
hsa-miR-129*:
(SEQ ID NO: 806)
AAGCCCUUACCCCAAAAAGUAU,
hsa-miR-129-3p:
(SEQ ID NO: 807)
AAGCCCUUACCCCAAAAAGCAU,
hsa-miR-1469:
(SEQ ID NO: 808)
CUCGGCGCGGGGCGCGGGCUCC,
hsa-miR-18b*:
(SEQ ID NO: 809)
UGCCCUAAAUGCCCCUUCUGGC,
hsa-miR-1909:
(SEQ ID NO: 810)
CGCAGGGGCCGGGUGCUCACCG,
hsa-miR-193b*:
(SEQ ID NO: 811)
CGGGGUUUUGAGGGCGAGAUGA,
hsa-miR-3154:
(SEQ ID NO: 812)
CAGAAGGGGAGUUGGGAGCAGA,
hsa-miR-3972:
(SEQ ID NO: 813)
CUGCCAGCCCCGUUCCAGGGCA,
hsa-miR-4259:
(SEQ ID NO: 814)
CAGUUGGGUCUAGGGGUCAGGA,
hsa-miR-4449:
(SEQ ID NO: 815)
CGUCCCGGGGCUGCGCGAGGCA,
hsa-miR-4652-3p:
(SEQ ID NO: 816)
GUUCUGUUAACCCAUCCCCUCA,
hsa-miR-4655-5p:
(SEQ ID NO: 817)
CACCGGGGAUGGCAGAGGGUCG,
hsa-miR-4664-5p:
(SEQ ID NO: 818)
UGGGGUGCCCACUCCGCAAGUU,
hsa-miR-4688:
(SEQ ID NO: 819)
UAGGGGCAGCAGAGGACCUGGG,
hsa-miR-4707-3p:
(SEQ ID NO: 820)
AGCCCGCCCCAGCCGAGGUUCU,
hsa-miR-4723-3p:
(SEQ ID NO: 821)
CCCUCUCUGGCUCCUCCCCAAA,
hsa-miR-4725-3p:
(SEQ ID NO: 822)
UGGGGAAGGCGUCAGUGUCGGG,
hsa-miR-4749-5p:
(SEQ ID NO: 823)
UGCGGGGACAGGCCAGGGCAUC,
hsa-miR-4769-3p:
(SEQ ID NO: 824)
UCUGCCAUCCUCCCUCCCCUAC,
hsa-miR-484:
(SEQ ID NO: 825)
UCAGGCUCAGUCCCCUCCCGAU,
hsa-miR-491-5p:
(SEQ ID NO: 826)
AGUGGGGAACCCUUCCAUGAGG,
hsa-miR-5008-5p:
(SEQ ID NO: 827)
UGAGGCCCUUGGGGCACAGUGG,
hsa-miR-5010-3p:
(SEQ ID NO: 828)
UUUUGUGUCUCCCAUUCCCCAG,
hsa-miR-5194:
(SEQ ID NO: 829)
UGAGGGGUUUGGAAUGGGAUGG,
hsa-miR-663:
(SEQ ID NO: 830)
AGGCGGGGCGCCGCGGGACCGC,
hsa-miR-744:
(SEQ ID NO: 831)
UGCGGGGCUAGGGCUAACAGCA,
hsa-miR-92a-2*:
(SEQ ID NO: 832)
GGGUGGGGAUUUGUUGCAUUAC,
hsa-miR-1247:
(SEQ ID NO: 833)
ACCCGUCCCGUUCGUCCCCGGA,
hsa-miR-1914*:
(SEQ ID NO: 834)
GGAGGGGUCCCGCACUGGGAGG,
hsa-miR-23a*:
(SEQ ID NO: 835)
GGGGUUCCUGGGGAUGGGAUUU,
hsa-miR-659:
(SEQ ID NO: 836)
CUUGGUUCAGGGAGGGUCCCCA,
hsa-miR-6722-3p:
(SEQ ID NO: 837)
UGCAGGGGUCGGGUGGGCCAGG,
mmu-miR-711:
(SEQ ID NO: 838)
GGGACCCGGGGAGAGAUGUAAG,
mmu-miR-762:
(SEQ ID NO: 839)
GGGGCUGGGGCCGGGACAGAGC,
hsa-miR-5196-5p:
(SEQ ID NO: 840)
AGGGAAGGGGACGAGGGUUGGG,
hsa-miR-155:
(SEQ ID NO: 841)
UUAAUGCUAAUCGUGAUAGGGGU,
hsa-miR-3184-3p:
(SEQ ID NO: 842)
AAAGUCUCGCUCUCUGCCCCUCA,
hsa-miR-3188:
(SEQ ID NO: 843)
AGAGGCUUUGUGCGGAUACGGGG,
hsa-miR-3191-3p:
(SEQ ID NO: 844)
UGGGGACGUAGCUGGCCAGACAG,
hsa-miR-3675-5p:
(SEQ ID NO: 845)
UAUGGGGCUUCUGUAGAGAUUUC,
hsa-miR-373:
(SEQ ID NO: 846)
GAAGUGCUUCGAUUUUGGGGUGU,
hsa-miR-423-3p:
(SEQ ID NO: 847)
AGCUCGGUCUGAGGCCCCUCAGU,
hsa-miR-423-5p:
(SEQ ID NO: 848)
UGAGGGGCAGAGAGCGAGACUUU,
hsa-miR-4698:
(SEQ ID NO: 849)
UCAAAAUGUAGAGGAAGACCCCA,
hsa-miR-5705:
(SEQ ID NO: 850)
UGUUUCGGGGCUCAUGGCCUGUG,
hsa-miR-6503-5p:
(SEQ ID NO: 851)
AGGUCUGCAUUCAAAUCCCCAGA,
hsa-miR-6511a-5p:
(SEQ ID NO: 852)
CAGGCAGAAGUGGGGCUGACAGG,
hsa-miR-6511b-3p:
(SEQ ID NO: 853)
CCUCACCACCCCUUCUGCCUGCA,
hsa-miR-767-3p:
(SEQ ID NO: 854)
UCUGCUCAUACCCCAUGGUUUCU,
mmu-miR-673-3p:
(SEQ ID NO: 855)
UCCGGGGCUGAGUUCUGUGCACC,
hsa-miR-3153:
(SEQ ID NO: 856)
GGGGAAAGCGAGUAGGGACAUUU,
hsa-miR-324-5p:
(SEQ ID NO: 857)
CGCAUCCCCUAGGGCAUUGGUGU,
hsa-miR-3679-5p:
(SEQ ID NO: 858)
UGAGGAUAUGGCAGGGAAGGGGA,
hsa-miR-4728-5p:
(SEQ ID NO: 859)
UGGGAGGGGAGAGGCAGCAAGCA,
hsa-miR-4741:
(SEQ ID NO: 860)
CGGGCUGUCCGGAGGGGUCGGCU,
hsa-miR-4758-3p:
(SEQ ID NO: 861)
UGCCCCACCUGCUGACCACCCUC,
hsa-miR-4758-5p:
(SEQ ID NO: 862)
GUGAGUGGGAGCCGGUGGGGCUG,
hsa-miR-4783-3p:
(SEQ ID NO: 863)
CCCCGGUGUUGGGGCGCGUCUGC,
hsa-miR-5090:
(SEQ ID NO: 864)
CCGGGGCAGAUUGGUGUAGGGUG,
hsa-miR-611:
(SEQ ID NO: 865)
GCGAGGACCCCUCGGGGUCUGAC,
hsa-miR-671-5p:
(SEQ ID NO: 866)
AGGAAGCCCUGGAGGGGCUGGAG,
hsa-miR-6721-5p:
(SEQ ID NO: 867)
UGGGCAGGGGCUUAUUGUAGGAG,
hsa-miR-769-3p:
(SEQ ID NO: 868)
CUGGGAUCUCCGGGGUCUUGGUU,
hsa-miR-3162-5p:
(SEQ ID NO: 869)
UUAGGGAGUAGAAGGGUGGGGAG,
hsa-miR-4707-5p:
(SEQ ID NO: 870)
GCCCCGGCGCGGGCGGGUUCUGG,
hsa-miR-4745-5p:
(SEQ ID NO: 871)
UGAGUGGGGCUCCCGGGACGGCG,
hsa-miR-623:
(SEQ ID NO: 872)
AUCCCUUGCAGGGGCUGUUGGGU,
hsa-miR-6724-5p:
(SEQ ID NO: 873)
CUGGGCCCGCGGCGGGCGUGGGG,
hsa-miR-3184-5p:
(SEQ ID NO: 874)
UGAGGGGCCUCAGACCGAGCUUUU,
hsa-miR-4697-3p:
(SEQ ID NO: 875)
UGUCAGUGACUCCUGCCCCUUGGU,
hsa-miR-5009-5p:
(SEQ ID NO: 876)
UUGGACUUUUUCAGAUUUGGGGAU,
hsa-miR-6511b-5p:
(SEQ ID NO: 877)
CUGCAGGCAGAAGUGGGGCUGACA,
hsa-miR-3137:
(SEQ ID NO: 878)
UCUGUAGCCUGGGAGCAAUGGGGU,
hsa-miR-4787-3p:
(SEQ ID NO: 879)
GAUGCGCCGCCCACUGCCCCGCGC,
hsa-miR-4649-5p:
(SEQ ID NO: 880)
UGGGCGAGGGGUGGGCUCUCAGAG,
hsa-miR-4763-3p:
(SEQ ID NO: 881)
AGGCAGGGGCUGGUGCUGGGCGGG,
hsa-miR-6089:
(SEQ ID NO: 882)
GGAGGCCGGGGUGGGGCGGGGCGG,
hsa-miR-3180-5p:
(SEQ ID NO: 883)
CUUCCAGACGCUCCGCCCCACGUCG,
hsa-miR-4706:
(SEQ ID NO: 884)
AGCGGGGAGGAAGUGGGCGCUGCUU,
hsa-miR-4728-3p:
(SEQ ID NO: 885)
CAUGCUGACCUCCCUCCUGCCCCAG,
hsa-miR-608:
(SEQ ID NO: 886)
AGGGGUGGUGUUGGGACAGCUCCGU,
hsa-miR-3189-5p:
(SEQ ID NO: 887)
UGCCCCAUCUGUGCCCUGGGUAGGA,
hsa-miR-4739:
(SEQ ID NO: 888)
AAGGGAGGAGGAGCGGAGGGGCCCU,
hsa-miR-4700-3p:
(SEQ ID NO: 889)
CACAGGACUGACUCCUCACCCCAGUG,
hsa-miR-1226*:
(SEQ ID NO: 890)
GUGAGGGCAUGCAGGCCUGGAUGGGG,
hsa-miR-4685-5p:
(SEQ ID NO: 891)
CCCAGGGCUUGGAGUGGGGCAAGGUU,
hsa-miR-1275:
(SEQ ID NO: 892)
GUGGGGGAGAGGCUGUC,
hsa-miR-4447:
(SEQ ID NO: 893)
GGUGGGGGCUGUUGUUU,
hsa-miR-3656:
(SEQ ID NO: 894)
GGCGGGUGCGGGGGUGG,
hsa-miR-1268:
(SEQ ID NO: 895)
CGGGCGUGGUGGUGGGGG,
hsa-miR-4253:
(SEQ ID NO: 896)
AGGGCAUGUCCAGGGGGU,
hsa-miR-4274:
(SEQ ID NO: 897)
CAGCAGUCCCUCCCCCUG,
hsa-miR-4278:
(SEQ ID NO: 898)
CUAGGGGGUUUGCCCUUG,
hsa-miR-4488:
(SEQ ID NO: 899)
AGGGGGCGGGCUCCGGCG,
hsa-miR-4327:
(SEQ ID NO: 900)
GGCUUGCAUGGGGGACUGG,
hsa-miR-4271:
(SEQ ID NO: 901)
GGGGGAAGAAAAGGUGGGG,
hsa-miR-6085:
(SEQ ID NO: 902)
AAGGGGCUGGGGGAGCACA,
hsa-miR-2392:
(SEQ ID NO: 903)
UAGGAUGGGGGUGAGAGGUG,
hsa-miR-3676-3p:
(SEQ ID NO: 904)
CCGUGUUUCCCCCACGCUUU,
hsa-miR-371-5p:
(SEQ ID NO: 905)
ACUCAAACUGUGGGGGCACU,
hsa-miR-3960:
(SEQ ID NO: 906)
GGCGGCGGCGGAGGCGGGGG,
hsa-miR-4749-3p:
(SEQ ID NO: 907)
CGCCCCUCCUGCCCCCACAG,
hsa-miR-6124:
(SEQ ID NO: 908)
GGGAAAAGGAAGGGGGAGGA,
hsa-miR-4313:
(SEQ ID NO: 909)
AGCCCCCUGGCCCCAAACCC,
hsa-miR-6716-5p:
(SEQ ID NO: 910)
UGGGAAUGGGGGUAAGGGCC,
hsa-miR-1202:
(SEQ ID NO: 911)
GUGCCAGCUGCAGUGGGGGAG,
hsa-miR-1237:
(SEQ ID NO: 912)
UCCUUCUGCUCCGUCCCCCAG,
hsa-miR-4687-3p:
(SEQ ID NO: 913)
UGGCUGUUGGAGGGGGCAGGC,
hsa-miR-5195-3p:
(SEQ ID NO: 914)
AUCCAGUUCUCUGAGGGGGCU,
hsa-miR-625:
(SEQ ID NO: 915)
AGGGGGAAAGUUCUAUAGUCC,
mmu-miR-715:
(SEQ ID NO: 916)
CUCCGUGCACACCCCCGCGUG,
mmu-miR-721:
(SEQ ID NO: 917)
CAGUGCAAUUAAAAGGGGGAA,
hsa-miR-1228*:
(SEQ ID NO: 918)
GUGGGCGGGGGCAGGUGUGUG,
hsa-miR-4433-3p:
(SEQ ID NO: 919)
ACAGGAGUGGGGGUGGGACAU,
mmu-miR-680:
(SEQ ID NO: 920)
GGGCAUCUGCUGACAUGGGGG,
hsa-miR-149*:
(SEQ ID NO: 921)
AGGGAGGGACGGGGGCUGUGC,
hsa-miR-6069:
(SEQ ID NO: 922)
GGGCUAGGGCCUGCUGCCCCC,
hsa-miR-940:
(SEQ ID NO: 923)
AAGGCAGGGCCCCCGCUCCCC,
hsa-miR-150*:
(SEQ ID NO: 924)
CUGGUACAGGCCUGGGGGACAG,
hsa-miR-1913:
(SEQ ID NO: 925)
UCUGCCCCCUCCGCUGCUGCCA,
hsa-miR-3020*:
(SEQ ID NO: 926)
UUUAACAUGGGGGUACCUGCUG,
hsa-miR-3675-3p:
(SEQ ID NO: 927)
CAUCUCUAAGGAACUCCCCCAA,
hsa-miR-373*:
(SEQ ID NO: 928)
ACUCAAAAUGGGGGCGCUUUCC,
hsa-miR-4689:
(SEQ ID NO: 929)
UUGAGGAGACAUGGUGGGGGCC,
hsa-miR-4697-5p:
(SEQ ID NO: 930)
AGGGGGCGCAGUCACUGACGUG,
hsa-miR-4716-3p:
(SEQ ID NO: 931)
AAGGGGGAAGGAAACAUGGAGA,
hsa-miR-4716-5p:
(SEQ ID NO: 932)
UCCAUGUUUCCUUCCCCCUUCU,
hsa-miR-4731-3p:
(SEQ ID NO: 933)
CACACAAGUGGCCCCCAACACU,
hsa-miR-4731-5p:
(SEQ ID NO: 934)
UGCUGGGGGCCACAUGAGUGUG,
hsa-miR-5010-5p:
(SEQ ID NO: 935)
AGGGGGAUGGCAGAGCAAAAUU,
hsa-miR-5698:
(SEQ ID NO: 936)
UGGGGGAGUGCAGUGAUUGUGG,
hsa-miR-625*:
(SEQ ID NO: 937)
GACUAUAGAACUUUCCCCCUCA,
mmu-miR-290-5p:
(SEQ ID NO: 938)
ACUCAAACUAUGGGGGCACUUU,
mmu-miR-292-5p:
(SEQ ID NO: 939)
ACUCAAACUGGGGGCUCUUUUG,
hsa-miR-1225-3p:
(SEQ ID NO: 940)
UGAGCCCCUGUGCCGCCCCCAG,
hsa-miR-4640-3p:
(SEQ ID NO: 941)
CACCCCCUGUUUCCUGGCCCAC,
hsa-miR-4787-5p:
(SEQ ID NO: 942)
GCGGGGGUGGCGGCGGCAUCCC,
hsa-miR-615-5p:
(SEQ ID NO: 943)
GGGGGUCCCCGGUGCUCGGAUC,
hsa-miR-4750-3p:
(SEQ ID NO: 944)
CCUGACCCACCCCCUCCCGCAG,
hsa-miR-361-3p:
(SEQ ID NO: 945)
UCCCCCAGGUGUGAUUCUGAUUU,
hsa-miR-3937:
(SEQ ID NO: 946)
ACAGGCGGCUGUAGCAAUGGGGG,
hsa-miR-3943:
(SEQ ID NO: 947)
UAGCCCCCAGGCUUCACUUGGCG,
hsa-miR-4665-5p:
(SEQ ID NO: 948)
CUGGGGGACGCGUGAGCGCGAGC,
hsa-miR-498:
(SEQ ID NO: 949)
UUUCAAGCCAGGGGGCGUUUUUC,
hsa-miR-4723-5p:
(SEQ ID NO: 950)
UGGGGGAGCCAUGAGAUAAGAGCA,
hsa-miR-637:
(SEQ ID NO: 951)
ACUGGGGGCUUUCGGGCUCUGCGU,
hsa-miR-939:
(SEQ ID NO: 952)
UGGGGAGCUGAGGCUCUGGGGGUG,
hsa-miR-1975:
(SEQ ID NO: 953)
CCCCCACAACCGCGCUUGACUAGCU,
hsa-miR-4665-3p:
(SEQ ID NO: 954)
CUCGGCCGCGGCGCGUAGCCCCCGCC,
hsa-miR-4472:
(SEQ ID NO: 955)
GGUGGGGGGUGUUGUUUU,
hsa-miR-4281:
(SEQ ID NO: 956)
GGGUCCCGGGGAGGGGGG,
hsa-miR-1228:
(SEQ ID NO: 957)
UCACACCUGCCUCGCCCCCC,
hsa-miR-6515-3p:
(SEQ ID NO: 958)
UCUCUUCAUCUACCCCCCAG,
hsa-miR-4525:
(SEQ ID NO: 959)
GGGGGGAUGUGCAUGCUGGUU,
hsa-miR-4433-5p:
(SEQ ID NO: 960)
CGUCCCACCCCCCACUCCUGU,
hsa-miR-3679-3p:
(SEQ ID NO: 961)
CUUCCCCCCAGUAAUCUUCAUC,
hsa-miR-1225-5p:
(SEQ ID NO: 962)
GUGGGUACGGCCCAGUGGGGGG,
hsa-miR-6087:
(SEQ ID NO: 963)
UGAGGCGGGGGGGCGAGC,
hsa-miR-6088:
(SEQ ID NO: 964)
AGAGAUGAAGCGGGGGGGCG,
hsa-miR-296-5p:
(SEQ ID NO: 965)
AGGGCCCCCCCUCAAUCCUGU,
and
hsa-miR-1249:
(SEQ ID NO: 966)
ACGCCCUUCCCCCCCUUCUUCA
(underlines indicate a target GC contiguous
sequence).

The term “target sequence” in the present specification is a sequence contained in a target nucleic acid to which a probe binds, and is a sequence having at least one target GC contiguous sequence. In another expression, the target sequence means a sequence intended to form a double strand with the probe of the present invention, that is, a sequence complementary to a fully complementary probe sequence. The target sequence may be a full length sequence of a target nucleic acid or a partial sequence of a target nucleic acid. For example, the target sequence may have 1 to 5, 1 to 4, 1 to 3, 1 to 2, or 1 target GC contiguous sequence. In a case where the target sequence has two or more target GC contiguous sequences, these target GC contiguous sequences may be present at a location separated from each other or may be present adjacent to each other as in the example of SEQ ID NO: 873 or SEQ ID NO: 965. A length of the target sequence is 10- to 50-mer. For example, a chain length of the target sequence of the present invention can be 10-mer or more, 11-mer or more, 12-mer or more, 13-mer or more, 14-mer or more, 15-mer or more, 16-mer or more, 17-mer or more, or 18-mer or more. In addition, a chain length of the target sequence of the present invention can be 50-mer or less, 45-mer or less, 40-mer or less, 35-mer or less, 30-mer or less, 29-mer or less, 28-mer or less, 27-mer or less, 26-mer or less, or 25-mer or less. A chain length of the target sequence of the present invention can be 10- to 40-mer, 13- to 30-mer, 15- to 28-mer, or 18- to 25-mer.

The term “non-target nucleic acid” in the present specification means a nucleic acid of which the presence is not intended to be detected by the probe of the present invention or is intended to be quantitatively determined by the probe of the present invention, and means a nucleic acid having the same GC contiguous sequence as that of a target sequence. The phrase “the same GC contiguous sequence as that of a target sequence” means the same sequence as the GC contiguous sequence that the target sequence has, and means a GC contiguous sequence longer or shorter by one to several bases than the GC contiguous sequence that the target sequence has.

In addition, the term “non-target sequence” means a sequence that a non-target nucleic acid has, and means a sequence having the same GC contiguous sequence as that of the target sequence.

In the present specification, the term “specificity” means a proportion of a probe that mistakenly did not bind to a non-target nucleic acid (negative) in a case of binding the probe to a target nucleic acid, and is represented by (the number of non-target nucleic acids that did not bind to probe)/(total number of non-target nucleic acids).

Furthermore, in the present specification, the term “false positive” means that a probe mistakenly binds to a non-target nucleic acid. In addition, the term “false positive rate” means a rate at which non-target nucleic acids are mistakenly detected as target nucleic acids, and is represented by 1-(specificity), or (the number of non-target nucleic acids to which probe is mistakenly bound)/(total number of non-target nucleic acids). In the present specification, the term “non-specific binding” means that the probe binds to non-target nucleic acids. The term “specific binding” refers to binding of the probe to a target nucleic acid without binding to a non-target nucleic acid. The phrase “the probe does not bind to non-target nucleic acids” is synonymous with “specificity is high” or “high specificity” and “a false positive rate is low”. For example, the phrase may mean that a rate of detecting (or quantitatively determining) non-target sequences as a false positive by the probe is low compared to a fully complementary probe, or mean specificity of 0.8, 0.9, 0.95, 0.98, 0.99, or 0.999.

### Advantageous Effects of Invention

The probe of the present invention enables detection or quantitative determination with a low false positive rate in nucleic acid detection, because binding to non-specific sequences occurs less as compared to a probe having a sequence fully complementary to a target sequence. In particular, the probe of the present invention can detect or quantitatively determine short-chain nucleic acids with high specificity by simple double-strand formation without requiring complex processes such as ligation and amplification, because the probe increases a difference in binding power between a target sequence and a non-target sequence by changing binding activity of the probe itself.

### BRIEF DESCRIPTION OF THE DRAWINGS

FIG. 1 shows a diagram in which A of the upper drawing represents a schematic view of a binding aspect with a fully complementary probe sequence (CCC) in a case where a target sequence is GGG, and B of the lower drawing represents a schematic view of a binding aspect with a probe (C*C) of the present invention.

FIG. 2A is a graph showing results of measuring a Tm value of binding between probe 1 (TCGCCCTCTCAACCCAGCTTTT (SEQ ID NO: 967) -Linker)) and a target/non-target sequence. On the graph, a left vertical axis represents standardized absorbance A (n, T), a right vertical axis represents a first derivative dA (n, T)/dT of the standardized absorbance, and a horizontal axis represents a measurement temperature T [ ° C.]. A solid line indicates standardized absorbance A (n, T), and a dotted line indicates a first derivative dA (n, T)/dT of the standardized absorbance.

FIG. 2B is a graph showing results of measuring a Tm value of binding between probe 2 (TCGCCCTCTCAAC*CAGCTTTT (SEQ ID NO: 968)-Linker) and a target/non-target sequence. A solid line, a dotted line, a left vertical axis, a right vertical axis, and a horizontal axis on the graph are the same as in FIG. 2A.

FIG. 2C is a graph showing results of measuring a Tm value of binding between probe 3 (TCGC*CTCTCAACCCAGCTTTT (SEQ ID NO: 969)-Linker) and a target/non-target sequence. A solid line, a dotted line, a left vertical axis, a right vertical axis, and a horizontal axis on the graph are the same as in FIG. 2A.

FIG. 2D is a graph showing results of measuring a Tm value of binding between probe 4 (TCGC*CTCTCAAC*CAGCTTTT (SEQ ID NO: 970)-Linker) and a target/non-target sequence. A solid line, a dotted line, a left vertical axis, a right vertical axis, and a horizontal axis on the graph are the same as in FIG. 2A.

FIG. 3A is a graph showing results of measuring a Tm value of binding between probe 23-mer and a target/non-target sequence. On the graph, a left vertical axis represents standardized absorbance A (n, T), a right vertical axis represents a first derivative dA (n, T)/dT of the standardized absorbance, and a horizontal axis represents a measurement temperature T [° C]. On the graph, a solid line indicates standardized absorbance A (n, T), and a dotted line indicates a first derivative dA (n, T)/dT of the standardized absorbance.

FIG. 3B is a graph showing results of measuring a Tm value of binding between probe 18-mer and a target/non-target sequence. A solid line, a dotted line, a left vertical axis, a right vertical axis, and a horizontal axis on the graph are the same as in FIG. 3A.

FIG. 3C is a graph showing results of measuring a Tm value of binding between probe 17-mer and a target/non-target sequence. A solid line, a dotted line, a left vertical axis, a right vertical axis, and a horizontal axis on the graph are the same as in FIG. 3A.

FIG. 4 is a view showing a surface of a working electrode in a state of being modified with a probe and HHT.

FIG. 5 is a view showing the surface of the working electrode in a state of being modified with the probe and HHT, and showing a marker in a state of reaching the surface of the working electrode.

FIG. 6 is a graph showing a CV waveform when the surface of the working electrode is modified with the probe and HHT. On the graph, a vertical axis indicates a current, and a horizontal axis indicates a voltage.

FIG. 7 shows results of adding only non-target sequences as a sample to the state of FIG. 6. It is a graph showing the surface of the working electrode in a state of being modified with the probe and HHT, and showing a CV waveform at the time of a marker in a state of reaching the surface of the working electrode, because no nucleic acid that hybridizes with the probe is present. On the graph, a vertical axis indicates a current, and a horizontal axis indicates a voltage.

FIG. 8 is a view showing a state in which the surface of the working electrode is modified with the probe and HHT, the probe and a nucleic acid are hybridized, and a marker is unlikely to reach the surface of the working electrode.

FIG. 9 is a graph showing a CV waveform at the time of a state in which the surface of the working electrode is modified with the probe and HHT, the probe and a nucleic acid are hybridized, and a marker is unlikely to reach the surface of the working electrode. On the graph, a vertical axis indicates a current, and a horizontal axis indicates a voltage.

FIG. 10A is a graph showing a CV waveform when each of a target sequence and a non-target sequence is modified with respect to probe 1. On the graph, a vertical axis indicates a current, and a horizontal axis indicates a voltage.

FIG. 10B is a graph showing a CV waveform when each of a target sequence and a non-target sequence is modified with respect to probe 2. On the graph, a vertical axis indicates a current, and a horizontal axis indicates a voltage.

FIG. 10C is a graph showing a CV waveform when each of a target sequence and a non-target sequence is modified with respect to probe 3. On the graph, a vertical axis indicates a current, and a horizontal axis indicates a voltage.

FIG. 10D is a graph showing a CV waveform when each of a target sequence and a non-target sequence is modified with respect to probe 4. On the graph, a vertical axis indicates a current, and a horizontal axis indicates a voltage.

FIG. 11A is a graph showing a CV waveform when each of a target sequence and a non-target sequence is modified with respect to probe 23-mer. On the graph, a vertical axis indicates a current, and a horizontal axis indicates a voltage.

FIG. 11B is a graph showing a CV waveform when each of a target sequence and a non-target sequence is modified with respect to probe 18-mer. On the graph, a vertical axis indicates a current, and a horizontal axis indicates a voltage.

FIG. 11C is a graph showing a CV waveform when each of a target sequence and a non-target sequence is modified with respect to probe 17-mer. On the graph, a vertical axis indicates a current, and a horizontal axis indicates a voltage.

### DESCRIPTION OF EMBODIMENTS

1. Method for Designing Polynucleobase Probe

In one embodiment, the present invention relates to a method for designing a polynucleobase probe sequence that is capable of binding, with high specificity, to a target nucleic acid having a target sequence having at least one sequence of SEQ ID NOs: 1 to 10 (a GC contiguous sequence), the method including:

A) selecting a 10- to 50-mer sequence fully complementary to the target sequence as a fully complementary probe sequence; and

B) (i) in the fully complementary probe sequence, designing the polynucleobase probe by substituting or making at least one of bases abasic in a portion complementary to the GC contiguous sequence in the target sequence, and/or

B) (ii) in the fully complementary probe sequence, designing the polynucleobase probe sequence by cleaving the fully complementary probe sequence such that a portion complementary to the GC contiguous sequence in the target sequence becomes 2 bases or less.

Preferably, the polynucleobase probe sequence of the present invention is designed by creating an abasic site, substituting, or cleaving such that the number of bases in which any one of guanine or cytosine is contiguous becomes two bases or less, in the polynucleobase sequence (a probe GC contiguous sequence) complementary to a target GC contiguous sequence in the probe sequence.

In the method of the present invention, a target sequence is contained in a target nucleic acid, and any sequence containing at least one GC contiguous sequence can be selected. A length of the target sequence is not particularly limited as long as specific detection of a target nucleic acid is possible, and can be a length of the target sequence described above. The fully complementary probe sequence can be obtained as a polynucleobase sequence that is fully complementary to the target sequence.

As a position and the number of bases to become abasic or be substituted, it is possible to adopt any of substitution/abasic site in the above-mentioned probe GC contiguous sequence of the present invention. “Abasic site creation” can be carried out by substituting a base site with a hydrogen atom, a hydroxyl group, a lower alkyl group, a lower acyl group or the like. In a case of PNA, creating an abasic site may be carried out by substituting a nitrogen atom of a glycine skeleton with a carbon atom (which may have a lower acyl group (such as an acetyl group) or a lower alkyl group (such as a methyl group) as a substituent). In addition, “substitution” can be carried out by substituting a base site with a non-complementary base (such as a natural base or an artificial base), or may be carried out by substituting a base site with a group such as a phenyl group and an anthraquinone group, which does not inhibit the formation of a double helix structure with another base. A substituted/abasic probe sequence is preferably designed to not to have a probe GC contiguous sequence.

Cleavage of a fully complementary probe sequence is carried out by cleaving the fully complementary probe sequence within a probe GC contiguous sequence. Cleavage is performed at a position at which a fragment intended to be utilized as a polynucleobase probe has, at the end thereof, two or less bases of guanine or cytosine derived from a probe GC contiguous sequence, among fragments obtained by cleavage. Accordingly, a polynucleobase probe obtained by cleavage has one or two bases of guanine or one or two bases of cytosine at one or both ends.

In a case where the fully complementary probe sequence has two or more probe GC contiguous sequences, a polynucleobase probe sequence may be designed by performing the substitution, creating an abasic site, and cleavage described above alone or in combination at the two or more GC contiguous sequences. The substitution, creating an abasic site, and cleavage are preferably performed in all probe GC contiguous sequences in the probe in the design of the present invention. Accordingly, the polynucleobase probe sequence is preferably designed to not to have a probe GC contiguous sequence.

In the present specification, “binding, detecting, or quantitatively determining with high specificity” or “specifically detecting/binding” a target sequence having at least one GC contiguous sequence means detection (or quantitative determination) of a non-target sequence as a false positive at a low rate, compared to a probe (in which substituting or creating an abasic site has not been performed) complementary to a target sequence (a probe having a fully complementary probe sequence, hereinafter referred to as a “fully complementary probe”). Regarding whether or not a rate at which a test probe detects (or quantitatively determines) non-target sequences as false positives is low compared to a fully complementary probe, for example, in a case where a Tm value of the test probe is lower than a Tm value of the fully complementary probe by measuring a temperature at which 50% of double strands dissociate into single strands (melting temperature: Tm value) when binding of both probes to non-target sequences; or in a case where a Tm value of the test probe cannot be measured (no formation of a double strand), it can be determined that a rate at which the test probe detects (or quantitatively determines) non-target sequences as false positives is low compared to the fully complementary probe. Alternatively, this may mean binding, detecting, or quantitatively determining with a specificity of 0.8, 0.9, 0.95, 0.98, 0.99, 0.999, or the like.

2. Production of Polynucleobase Probe

As the polynucleobase probe according to the present invention, a probe designed by the above-described design method can be produced by utilizing methods known in the technical field. In particular, methods of chemical synthesis in which polynucleobases such as DNA/RNA, PNA, and LNA are bound one by one are well known, and such methods can be adopted. For example, in a case of PNA, using an Fmoc solid phase synthesis method, a base can be substituted with a carbon skeleton by elongating with 5-[(9-Fluorenylmethoxycarbonyl)amino] pentanoic acid, instead of elongating a site to become abasic with a base. In addition, if necessary, a synthesized polynucleobase probe can be bound to a modifier such as a solid phase or a label.

3. Method for Detecting or Quantitatively Determining Target Nucleic Acid having GC Contiguous Sequence using Polynucleobase Probe According to the Present Invention

In another aspect, the present invention relates to a method for detecting a target nucleic acid having at least one sequence of any one of SEQ ID NOs: 1 to 10 in a test sample with high specificity, the method including:

preparing the test sample to detect the target nucleic acid;

contacting at least one kind of the polynucleobase probes according to the present invention with the test sample; and

detecting the target nucleic acid bound to the polynucleobase probe.

In another aspect, the present invention relates to a method for quantitatively determining a target nucleic acid having at least one sequence of any one of SEQ ID NOs: 1 to 10 in a test sample with high specificity, the method including:

preparing the test sample to quantitatively determine the target nucleic acid;

contacting at least one kind of the polynucleobase probes according to the present invention with the test sample; and

quantitatively determining the target nucleic acid bound to the polynucleobase probe.

In the detection method and quantitative determination method of the present invention, a test sample can be prepared by using a target sample from which the presence or an amount of a target nucleic acid that is to be detected or quantitatively determined is examined. For example, for diagnostic purposes, the target sample is not particularly limited as long as it is a sample from which DNA or RNA can be detected, and it is possible to use body fluids or tissues such as lymph fluid, blood (serum, plasma), urine, feces, saliva, spinal fluid, tears, biopsy, hair, skin, nails, leachates, and cells (such as circulating tumor cells in blood (CTC)); exosomes; or cell-free DNA. These samples are appropriately prepared as samples suitable for detection of DNA or RNA.

Contact between at least one type of the polynucleobase probes according to the present invention and the test sample can be performed by, for example, mixing the polynucleobase probe according to the present invention and the test sample in a buffer. In particular, in a case where the polynucleobase probe according to the present invention is bound to a solid phase, the polynucleobase probe can be brought into contact in a static state, or can be dynamically brought into contact by a microfluidic device or the like.

For detection or quantitative determination of a target nucleic acid bound to a polynucleobase probe, methods widely known in the field of nucleic acid detection can be adopted. In a case where a label is bound to the probe, a detection method and a quantitative determination method can be adopted according to the type of the label. In addition, in a case where the label is not bound to the probe, it is possible to perform electrical detection or quantitative determination by using an intercalating agent inserted into a double strand (refer to, for example, Japanese Unexamined Patent Publication No. 2006-061061).

4. Another Method for Utilizing Polynucleobase Probe According to the Present Invention

In addition to the detection and quantitative determination methods in which direct measurement is performed after hybridization as described above, the probe of the present invention can also be used in detection and quantitative determination methods by utilizing amplification and ligation after utilizing hybridization. In particular, in a case of utilizing amplification, the probe can be used as a primer. In addition, the probe of the present invention can be used as an antisense DNA. Such an antisense DNA can be used for knockout/knockdown of gene expression. Furthermore, such an antisense DNA can be used for therapeutic purposes, for example, for gene therapy.

For example, the probe according to the present invention can be used in the following method:

A method for detecting or quantitatively determining a target nucleic acid in a test sample with high specificity, compared to detection and quantitative determination which use a probe (primer) complementary to a target sequence having at least one sequence of any one of SEQ ID NOs: 1 to 10, the method including:

preparing the test sample to quantitatively determine the target nucleic acid;

contacting at least one kind of the polynucleobase probes (primers) according to the present invention with the test sample;

amplifying a nucleic acid complementary to the target nucleic acid; and

detecting or quantitatively determining the amplified target nucleic acid.

Alternatively, the probe according to the present invention can be used in the following method:

A method for detecting or quantitatively determining a target nucleic acid in a test sample with high specificity, compared to detection and quantitative determination which use a probe complementary to a target sequence having at least one sequence of any one of SEQ ID NOs: 1 to 10, the method including:

preparing the test sample to quantitatively determine the target nucleic acid;

contacting, with the test sample, (i) at least one type of the polynucleobase probes according to the present invention, and (ii) a complementary probe that does not have a target sequence overlapping with the polynucleobase probe according to the present invention, in which bases separated by one to several bases from the target sequence of the polynucleobase probe according to the present invention are at the end of the target sequence;

binding by ligation of two types of probes forming a complementary strand with the target nucleic acid; and

detecting or quantitatively determining a bound substance of the two types of probes.

### EXAMPLES

Hereinafter, the present invention will be specifically explained based on examples, but the present invention is not limited thereto. The present application claims priority right based on Japanese Patent Application No. 2017-030553 filed Feb. 22, 2017, and the contents described in the application are incorporated in the present specification by reference in their entirety. In addition, the contents described in all of the patents, patent applications, and documents cited in the present application are incorporated in the present specification by reference in their entirety.

### Example 1

### Tm Value Determination Method

Absorbance at 260 nm and 320 nm of each cell was measured while changing a temperature, and a Tm value of a double strand formed by a probe and a target was determined from the obtained data of absorbance with respect to a temperature. A measuring device of an amount of change in absorbance, and conditions were as follows. Absorbance measurements were performed during annealing and Tm value measurements according to measurement software settings.

Measuring Device

Spectrophotometer UV-2600 (manufactured by Shimadzu Corporation)

Temperature controller TMSPC-8 (manufactured by Shimadzu Corporation)

8 Multicell 208-92097-11 (manufactured by Shimadzu Corporation) Measurement software UVProbe ver. 2.52 (manufactured by Shimadzu Corporation)

Constant Temperature Bath CCA-1111 (manufactured by EYERA)

Measurement software setting

Standby time before absorbance measurement: 4 minutes

Absorbance measurement interval: 1° C.

Slit width: 1.0 nm

Cumulative time: 3

Temperature blank

SSC (1×)

20% DMSO aqueous solution

Measurement sample

SSC (1×)

20% DMSO aqueous solution

Probe 2 μM

Target 2 μM

SSC: Saline Sodium Citrate Buffer

Specifically, cells to which a nucleic acid sample in which each of probes/targets to be measured are combined, or a temperature blank is added were allowed to stand by before annealing at 95° C. for 10 minutes, and then cooled from 95° C. to 20° C. at 0.5° C./min to be annealed. Thereafter, after standing by before Tm value measurement at 20° C. for 60 minutes, the temperature was raised from 20° C. to 95° C. at 0.5° C./min to measure the Tm value. A baseline was measured using a temperature blank. Using the data of the temperature blank cell, baseline correction was performed on a wavelength range of 330 nm to 250 nm.

Based on the obtained absorbance at two wavelengths (260 nm and 320 nm), two-wavelength correction was performed by subtracting environment-dependent absorbance variation A320 (n, T) from absorbance A260 (n, T) of the nucleic acid in the sample, and temperature-corrected absorbance Aw was calculated.

Aw (n, T)=A260(n, T)−A320(n, T)

n: Cell number (n=1, 2, . . . , 8)

T: Temperature at measurement (T =20, 21, . . . , 95)

Aw (n, T): Temperature-corrected absorbance at temperature T of cell number n

A260 (n, T): Absorbance at a wavelength of 260 nm at temperature T of cell number n

A320 (n, T): Absorbance at a wavelength of 320 nm at temperature T of cell number n

Furthermore, in order to remove the absorbance variation due to the temperature change of the solvent, temperature correction was performed by subtracting Aw (1, T) of the temperature blank cell from Aw (n, T) of the nucleic acid sample, and temperature-blank-corrected absorbance At was calculated.

At (n, T)=Aw (n, T)−Aw (1, T)

At (n, T): Temperature-blank-corrected absorbance of cell number n

Standardized absorbance A was calculated by standardizing the obtained At (n, T) such that a maximum value thereof became 1. Accordingly, Max (A (n, T))=1.

A (n, T)=At (n, T)/Max (At (n, T))

A (n, T): Standardized absorbance

Max (At (n, T)): Maximum value of cell number n at At (n, T)

Based on A (n, T) obtained by the above calculation, an amount of change in absorbance [%] Δ A abs (n) of cell number n showing an amount of base pairs formed, and a maximum value Tm (n) of a first derivative due to a temperature of cell number n which shows a Tm value [° C.] were obtained according to the following equation. In the following equation, Min (At (n, T)) represents a minimum value at At (n, T) of cell number n.

Δabs (n)=(Max(A(n, T))−Min (A(n, T)))*100

Tm (n)=Max (dA (n, T)/dT)

### Example 2

### Abasic Probe

Abasic site creation was performed by substituting a part of the probe using PNA which is one embodiment of the present invention with 5-aminopentanoic acid (hereinafter Ape). Hereinafter, the term “Linker” represents a structure including a thiol group for binding a probe to a gold electrode. A target GC contiguous sequence in the present example was GGG, and the probe GC contiguous sequence was CCC. A substitution site with Ape is indicated by “*”.

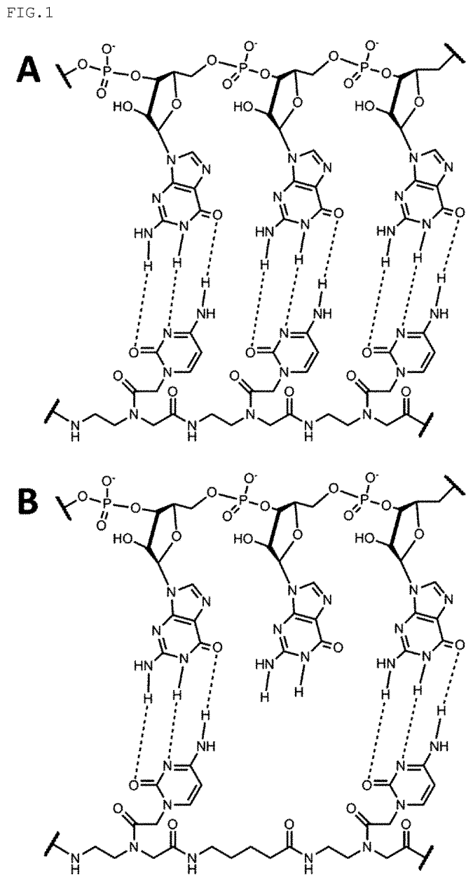

FIG. 1A shows a state in which no abasic site is contained, and in which CCC of a PNA probe (a lower part in the drawing) and GGG of a target RNA (an upper part in the drawing) form a base pair. FIG. 1B shows a state in which an abasic site by substitution of cytosine with Ape is applied, and in which C*C of a probe (a lower part in the drawing) and GGG of an RNA in the sample (an upper part in the drawing) do not form a base pair partially.

In order to confirm effects of abasic sites, using four types of probes 1 to 4 which have the following sequences, Tm values with respect to base pair formation by a target sequence (a sequence complementary to a probe) and a non-target sequence (a sequence not complementary to a probe) were measured by the method described above. Non-target sequences have sequences that are not complementary to the probe, but contain the same GC contiguous sequence (GGG) as the target sequence. For this reason, non-target sequences are likely to form base pairs with the probes, and base pair formation by non-target sequences and the probes means a false positive.

(Target Sequence and Non-Target Sequence)

(SEQ ID NO: 971)
Target sequence: AAAAGCUGGGUUGAGAGGGCGA
(SEQ ID NO: 972)
Non-target sequence: UGGCAGGGAGGCUGGGAGGGG
(Probe)
(SEQ ID NO: 967)
Probe 1: TCGCCCTCTCAACCCAGCTTTT-Linker
(SEQ ID NO: 968)
Probe 2: TCGOCCTCTCAAC*CAGCTTTT-Linker
(SEQ ID NO: 969)
Probe 3: TCGC*CTCTCAACCCAGCTTTT-Linker
(SEQ ID NO: 970)
Probe 4: TCGC*CTCTCAAC*CAGCTTTT-Linker

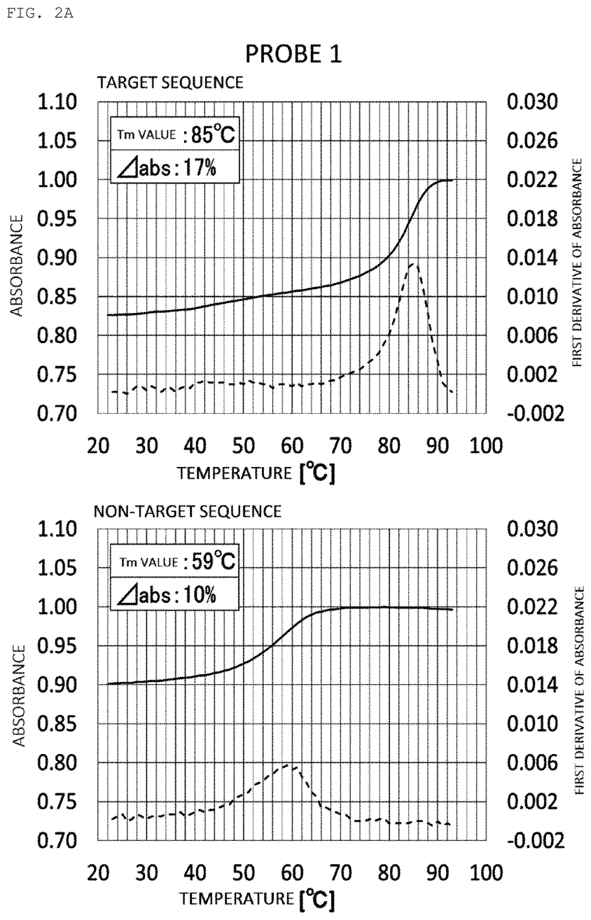

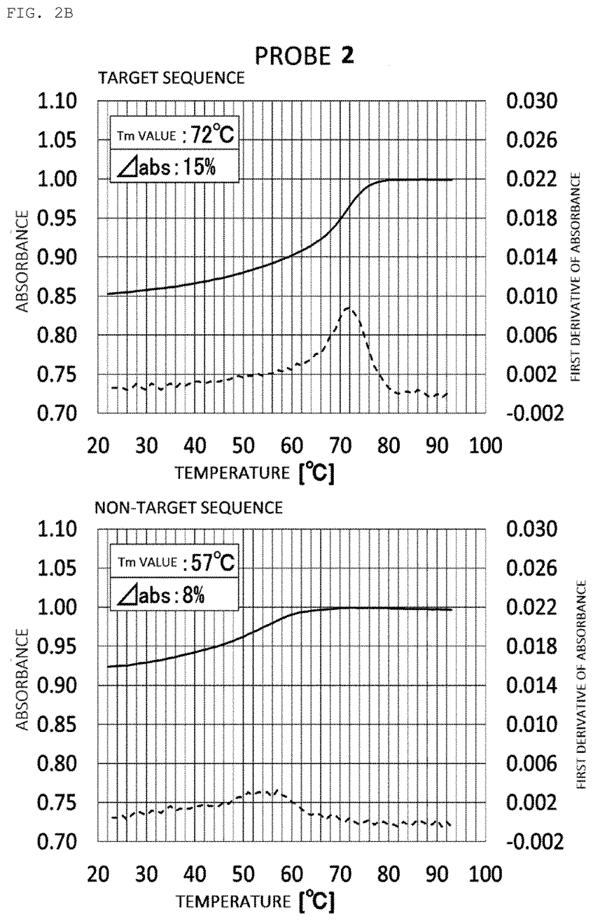

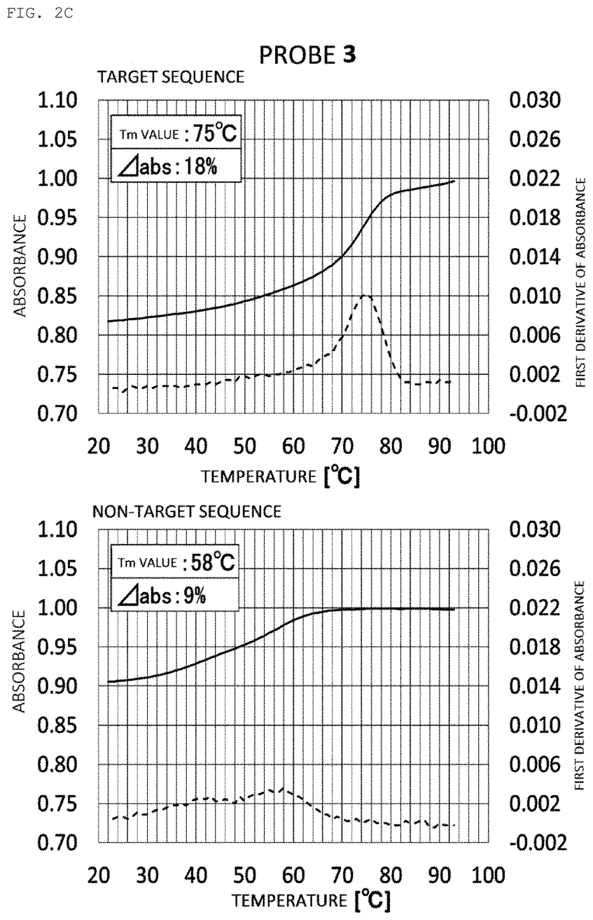

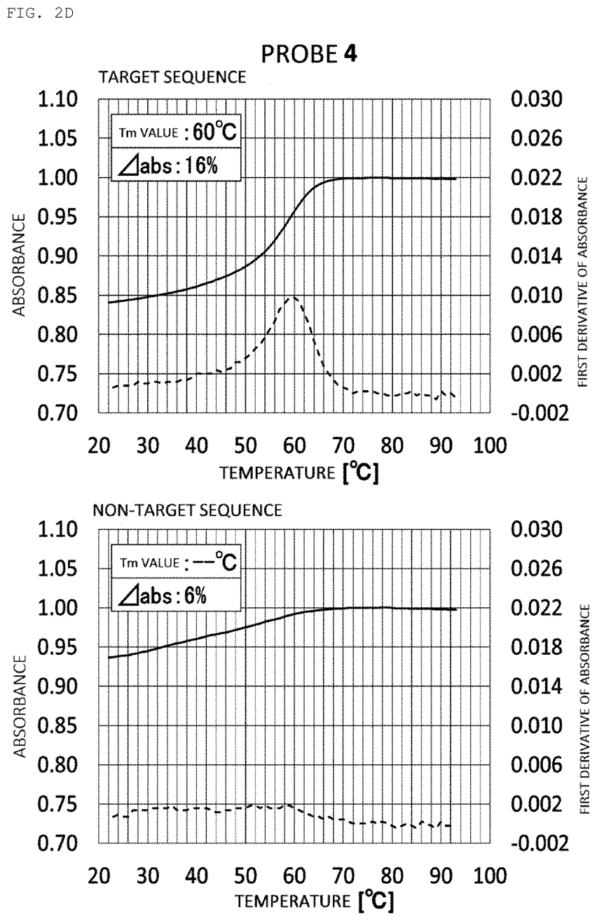

The results of measuring Tm values of eight combinations of the probe and the target/non-target sequences by the method shown in Example 1 are shown in FIGS. 2A to 2D. A Tm value for binding of probe 1 to a non-target sequence is 58° C. Aabs was as high as 10% which indicates that probe 1 formed a base pair with a non-target sequence which is originally a non-complementary strand. On the other hand, in the case of abasic probes 2, 3 and 4, a difference appeared in Aabs with respect to a non-target sequence, and a decreasing tendency was further observed in the probe 4 including two abasic sites which is compared with the probes 2 and 3 including one abasic site. It is shown that non-specific base pair formation (a false positive) by non-target sequences with target sequences having two or more GC contiguous sequences can be effectively reduced by using a probe (a site-2-substituted probe) in which a portion complementary to both GC contiguous sequences in the complementary probe is substituted with Ape. In probe 4, the variation of dA (n, T)/dT in base pair formation with a non-target sequence is a noise level, and a Tm value could not be obtained. Accordingly, probe 4 is perceived not to bind to a non-target sequence at any temperature. On the other hand, a Tm value for a target sequence tended to decrease in accordance with an increase of the abasic site, but in the two-sites-substituted probe, a Tm value was 58° C., and Aabs was also high. Based on the above description, it was confirmed that the double strand was formed with the target without problems. Therefore, it was shown that detection of a target sequence with a low false positive rate is possible by appropriately controlling a temperature using an abasic probe.

### Example 3

### Probes with Different Chain Lengths

As described in Example 2 above, it was shown that an abasic site of a GC contiguous sequence effectively enables detection with low false positive rate by an experiment using an abasic probe in which bases in the sequence are deleted. Based on the above description, it was examined whether or not detection with low false positive rate is possible with a probe in which a sequence length was shortened by cleaving in the middle of the GC contiguous sequence, in the sequence in which the GC contiguous sequence is present near the end.

Specifically, the GC contiguous sequence in the probe using PNA, which is one embodiment of the present invention, was cleaved, and three types of probes which have different chain lengths and have the following sequences were prepared. Tm values were measured by the method described above with respect to base pair formation by target sequences (sequences complementary to probes) and non-target sequences (sequences not complementary to probes). Non-target sequences have sequences that are not complementary to the probe, but contain the same GC contiguous sequence (GGG) as the target sequence. For this reason, non-target sequences are likely to form base pairs with the probes, and base pair formation with the probes means a false positive. A target GC contiguous sequence in the present example was GGG, and the complementary probe GC contiguous sequence was CCC.

(Target Sequence and Non-Target Sequence)

(SEQ ID NO: 973)
Target sequence: AGCUACAUUGUCUGCUGGGUUUC
(SEQ ID NO: 974)
Non-target sequence: UGGCAGGGAGGCUGGGAGGGG
(Probe)
(SEQ ID NO: 975)
Probe 23-mer: GAAACCCAGCAGACAATGTAGCT-Linker
(SEQ ID NO: 976)
Probe 18-mer: CCAGCAGACAATGTAGCT-Linker
(SEQ ID NO: 977)
Probe 17-mer: CAGCAGACAATGTAGCT-Linker

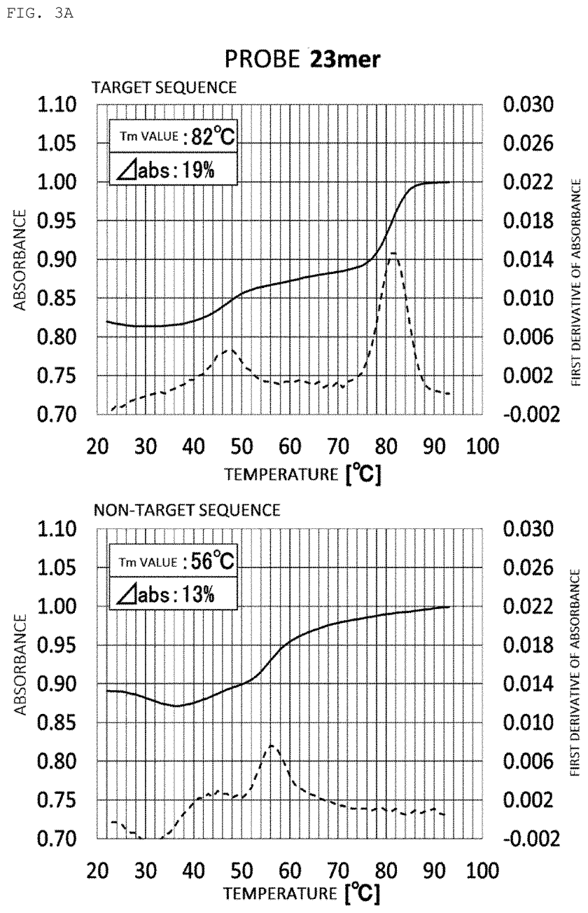

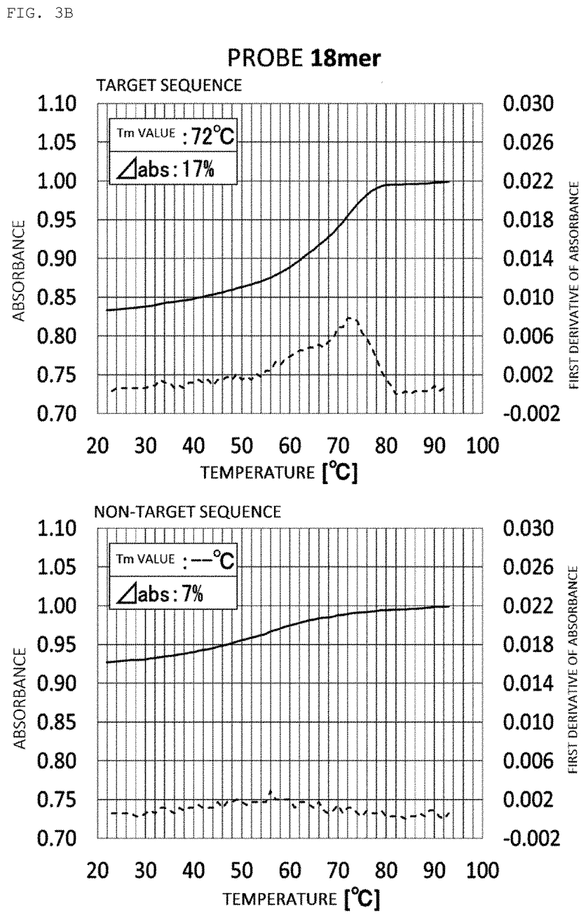

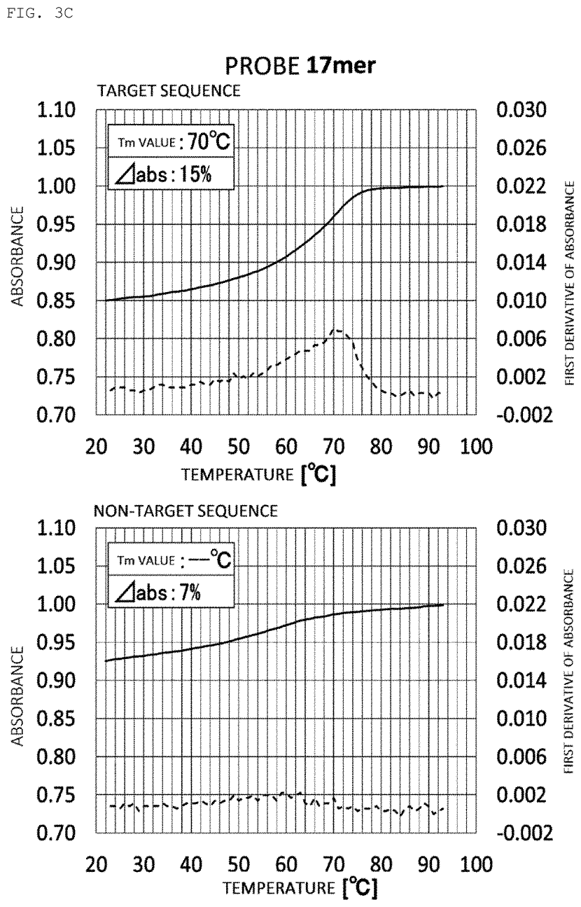

The results of measuring Tm values of six combinations of the probe and the target/non-target sequences by the method shown in Example 1 are shown in FIGS. 3A to 3C.

A large difference is shown in Aabs with respect to non-target sequences between probe 23-mer and probe 18-mer or probe 17-mer. However, no difference is shown between the probe 18-mer and the probe 17-mer, and it was shown that a base pair with a non-target sequence is hardly formed merely by making one base short from three consecutive cytosines. On the other hand, a Tm value for the target sequence tended to decrease as a chain length was shortened, but in the 17-mer probe, the Tm value is 70° C., and Aabs is also high. Accordingly, it was confirmed that a double strand was formed with the target. Therefore, it is shown that, in a case where GC contiguous sequences are present near the end of the probe or target sequence, an effect of reducing non-specific base pair formation (a false positive) with non-target sequences is exhibited by shortening of a probe chain length. In probe 18-mer and probe 17-mer, the variation of dA (n, T)/dT in base pair formation with a non-target sequence is a noise level, and a Tm value could not be obtained. Accordingly, probe 18-mer and probe 17-mer are perceived not to bind to a non-target sequence at any temperature. Therefore, it was shown that detection of a target sequence with a low false positive rate is possible by appropriately controlling a temperature using a short chain probe.

### Example 4

### Establishment of Method for Detecting Nucleic Acid by Electrochemical Measurement

In the present example, the term “modification” refers to a process of dropwise adding of a corresponding solution on a working electrode by a pipette and then allowing it to stand at a designated temperature for a designated time. In addition, in the present example, the term “washing” refers to a process of washing a surface of a gold electrode with a designated washing solution at a designated temperature.

(1) Adjustment of Measurement Solution

A pH of a sodium dihydrogen phosphate aqueous solution was adjusted to become 7.0 with sodium hydroxide, and then sodium perchlorate and potassium hexacyanoferrate (II) were added thereto. A final concentration of the measurement solution was 2.5 mM of sodium dihydrogen phosphate, 5 mM of sodium perchlorate, and 1 mM of potassium hexacyanoferrate (II).

(2) Measurement Method

The electrochemical measurement in the present example was performed by the following steps. In the following table, RT represents room temperature (about 25° C.), TFA represents trifluoroacetic acid, DMSO represents dimethyl sulfoxide, and Milli-Q represents ultra pure water.

TABLE 1

| | | | | | | Concen- | |
|---|---|---|---|---|---|---|---|
| | | Temper- | | Amount of | | tration of | |
| Process | Time | ature | Solvent | solution | | solute | |
| 1. Probe | 30 min. | 25° C. | 0.05% | 10 | ul | 10 | uM |
| modification | | | TFA aq | | | | |
| 2. Washing | | 80° C. | 20% | 100 | ml | | |
| | | | DMSO aq | | | | |
| 3. HHT | 30 min. | 25° C. | Milli-Q | 25 | ul | 1 | mM |
| modification | | | | | | | |
| 4. Washing | | RT | Milli-Q | 50 | ml | | |
| 5. Measure- | | RT | Measure- | 1 | ml | | |
| ment 1 | | | ment | | | | |
| | | | solution | | | | |
| 6. Sample | 40 min. | 40° C. | 1*SSC, | 250 | ul | 25 | nM |
| modification | | | 20% | | | | |
| | | | DMSO aq | | | | |

| | | | | |
|---|---|---|---|---|
| 7. Washing | RT | Milli-Q | 50 | ml |
| 8. Washing | 50° C. | Milli-Q | 100 | ml |
| 9. Measure- | RT | Measure- | 1 | ml |
| ment 1 | | ment (*1) | | |

As a sample, a solution containing any one of a target nucleic acid or a non-target nucleic acid which has a specified concentration was used. The measurement was carried out by cyclic voltammetry (hereinafter, CV) by using working electrode: gold electrode with a diameter of 300 μm, counter electrode: Pt counter electrode of 5 cm manufactured by BAS, reference electrode: RE-1B aqueous reference electrode (Ag/AgCl) manufactured by BAS, Potentiostat (miniSTAT 100 manufactured by BioDevice Technology). The measurement conditions (miniStat 100 setting contents) were as follows.

TABLE 2

| Control software | | | |
|---|---|---|---|
| parameter name | Setting value | | Commentary |
| Begin potential | −150 | mV | Initial voltage |
| First vertex potential | 500 | mV | Firstly reached voltage |
| Second vertex potential | −150 | mV | Secondly reached voltage |
| Interval time | 50 | ms | Measurement interval |
| Scan rate | 100 | mv/s | Voltage sweep rate |

| | | |
|---|---|---|
| Range | uA | Measurement voltage range |

(3) Measurement 1

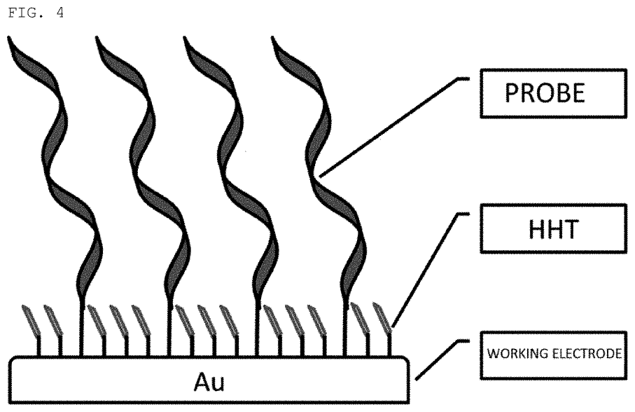

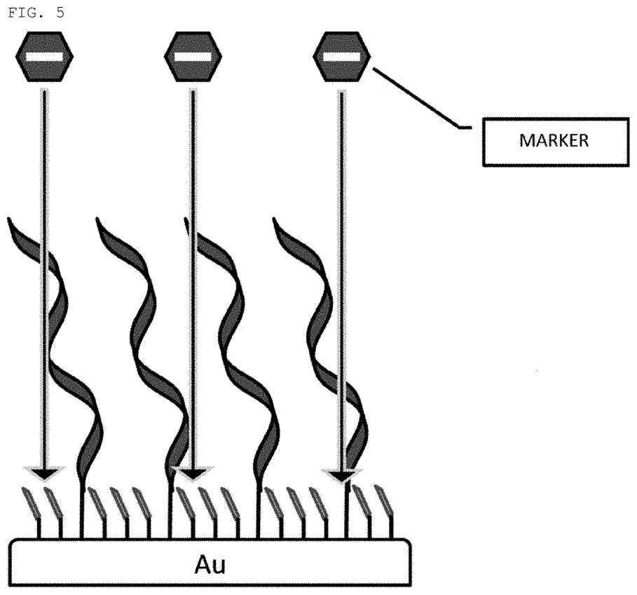

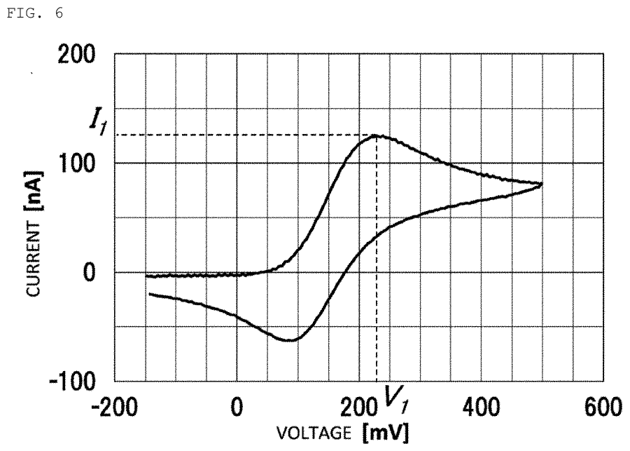

A surface of the working electrode was modified with a probe and 6-Hydroxy-1-hexanethiol (HHT) (FIG. 4). In this state, the surface of the working electrode is not charged. Therefore, in the measurement solution, hexacyanoferrate (II) ions (hereinafter referred to as a marker) can reach the surface of the working electrode according to a set potential (FIG. 5). A measured waveform by CV in this state was taken as a basic value (FIG. 6). In addition, a voltage value at which the maximum current value i1 was recorded in the measurement 1 is set to V1.

(4) Measurement 2: Non-Target Nucleic Acid

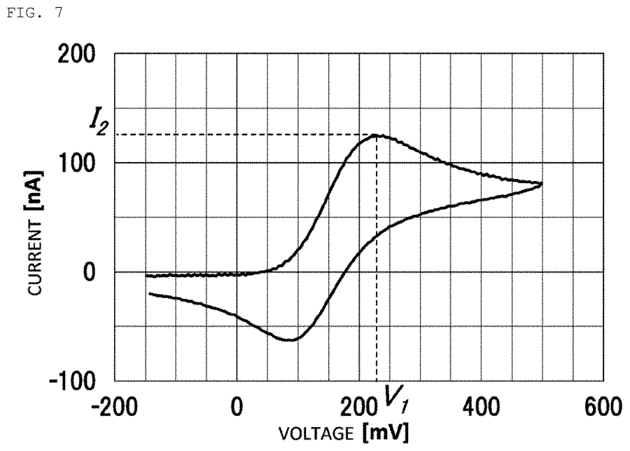

An electrode was modified with a sample containing a non-target nucleic acid (hereinafter, non-complementary electrode). The marker reached a surface of the electrode as in the initial state (FIG. 5), because the non-target nucleic acid hardly hybridized with the probe. Therefore, the same CV waveform (FIG. 7) as in measurement 1 was obtained in measurement 2. A current value i2 at voltage value v1 at which the maximum current value i1 was recorded in measurement 1 is theoretically the same value as the maximum current value i1 in measurement 1 when measurement errors are excluded.

(5) Measurement 2: Target Nucleic Acid

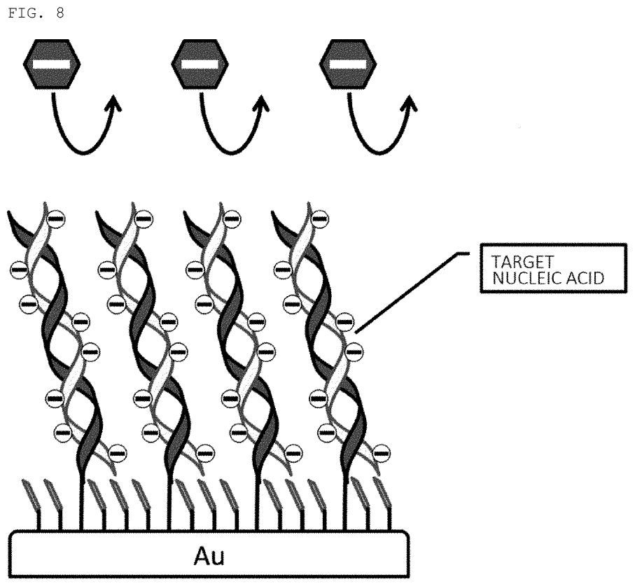

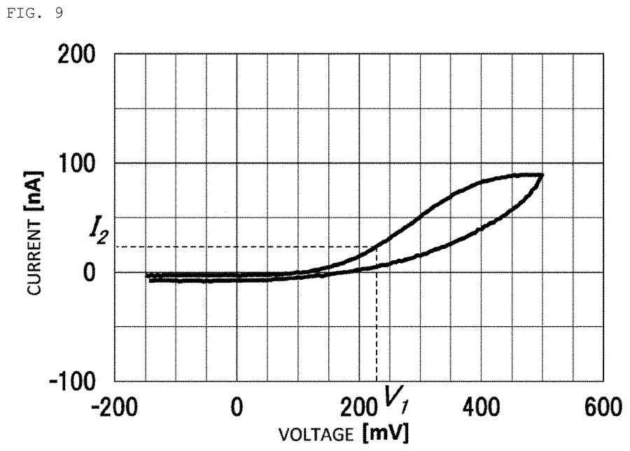

Next, an electrode was modified with a sample containing a target nucleic acid (hereinafter, complementary electrode). Because the probe and target nucleic acid hybridized, the marker received repulsion due to a negative charge of the nucleic acid, and therefore it was difficult for the marker to reach the surface of the electrode (FIG. 8). Therefore, in the CV waveform obtained, the current value i2 at the voltage value V1 at which the maximum current value i1 was recorded in measurement 1 decreased (FIG. 9).

(6) Determination of Hybridization Determination Method

Based on the above results, whether the measured electrode was a complementary electrode or a non-complementary electrode was determined from a current value at the voltage value V1 at which the maximum current value i1 was recorded in measurement 1. A current value ratio i2/i1 obtained in measurements 1 and 2 is ideally 1 for a case of non-complementary electrodes, and is smaller than 1 for a case of complementary electrodes. Practically, in consideration of measurement error, an electrode was determined to be a non-complementary electrode when a current value ratio was i2/i1≥0.9, and was determined to be a complementary electrode when a current value ratio was i2/i1<0.9.

### Example 5

### Evaluation of Influence on Nucleic Acid Detection by Abasic Site of GC Contiguous Sequence of Probe

Using the CV measurement method described above, measurements of hybridization of probes 1 to 4 with respect to the following target sequence and non-target sequences were performed.

(SEQ ID NO: 971)
Target sequence: AAAAGCUGGGUUGAGAGGGCGA
(SEQ ID NO: 972)
Non-target sequence: UGGCAGGGAGGCUGGGAGGGG
(Probe)
(SEQ ID NO: 967)
Probe 1: TCGCCCTCTCAACCCAGCTTTT-Linker
(SEQ ID NO: 968)
Probe 2: TCGCCCTCTCAAC*CAGCTTTT-Linker
(SEQ ID NO: 969)
Probe 3: TCGC*CTCTCAACCCAGCTTTT-Linker
(SEQ ID NO: 970)
Probe 4: TCGC*CTCTCAAC*CAGCTTTT-Linker

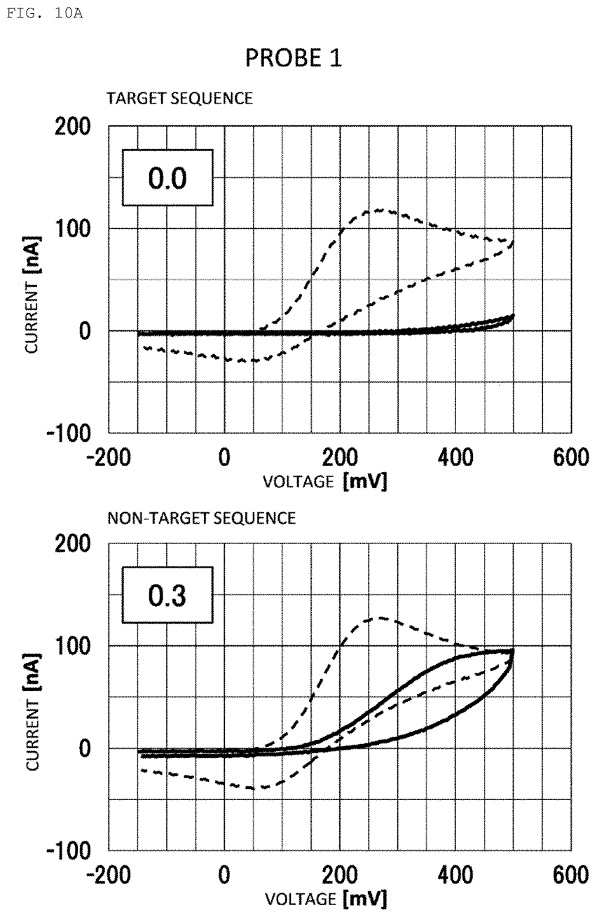

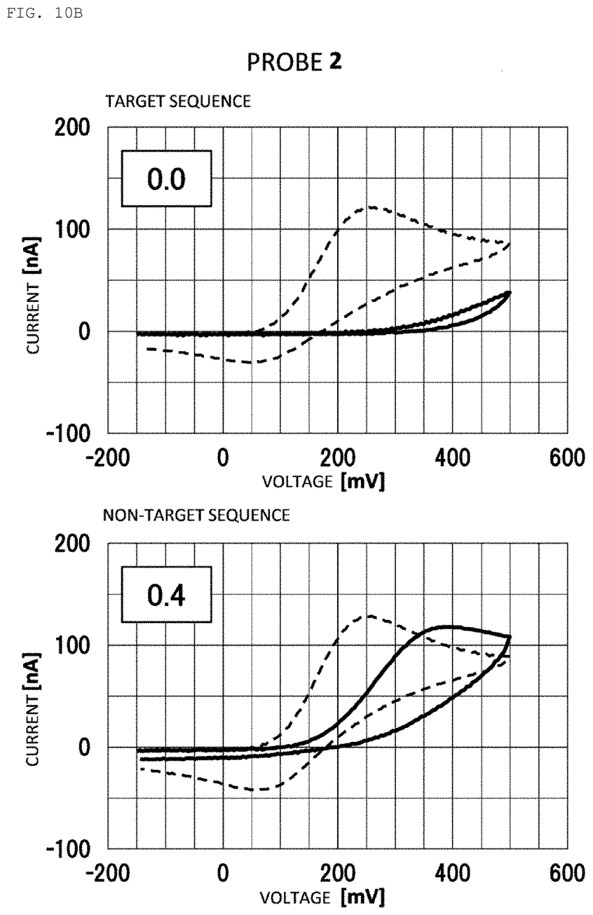

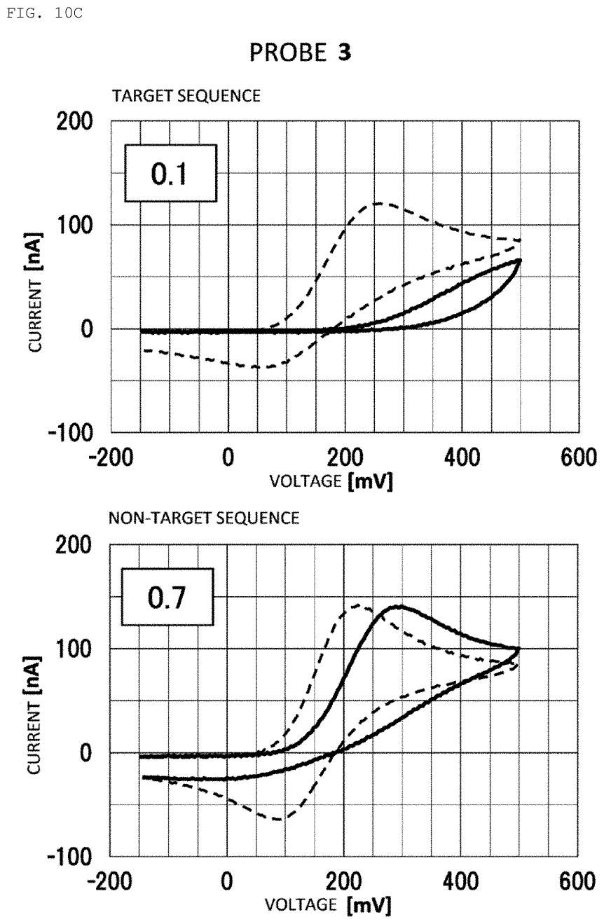

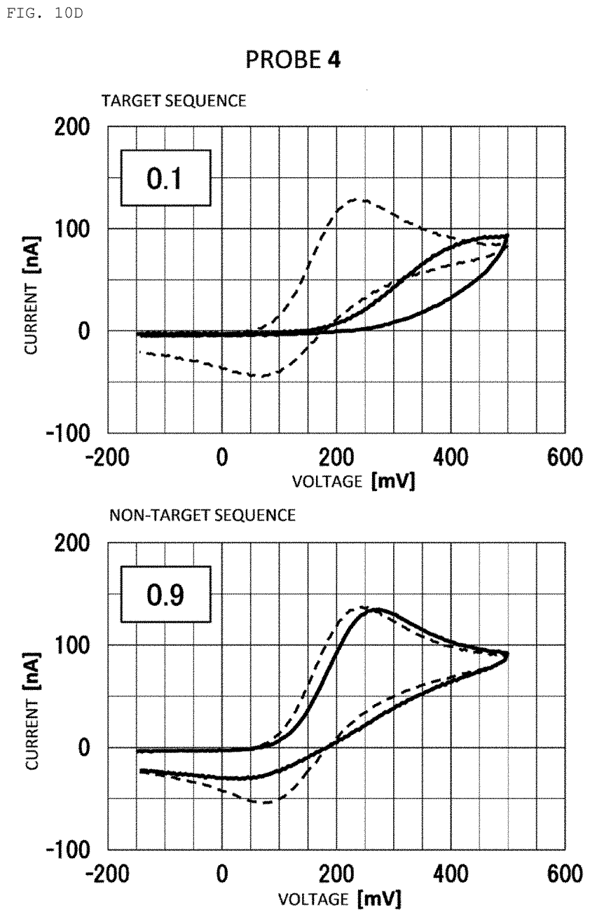

FIG. 10 shows a CV waveform when each of target sequences and non-target sequences is modified for each of probes. The CV waveform of measurement 1 is shown by a dotted line, and the CV waveform of measurement 2 is shown by a solid line, and the current value ratio is described at the upper left.

Target sequence and probes 1 to 4: Because all current value ratios of measurement 2 to measurement 1 decreased to 0.1 or less, hybridization was correctly performed with the target sequence.

Non-target sequence and probe 1: A current value ratio is 0.3, and mishybridization with the non-target sequence was caused.

Non-target sequence and probes 2 and 3: Current ratios are 0.4 and 0.7, which are higher than the current ratio of probe 1 which is 0.3, and therefore the effect of reducing mishybridization by making one site of the GC contiguous sequence abasic was observed.

Non-target sequence and probe 4: A current value ratio was 0.9 and did not cause mishybridization. The effect obtained by making two sites of the GC contiguous sequence abasic was observed.

### Example 6

### Evaluation of Influence on Nucleic Acid Detection by Cleavage of GC Contiguous Sequence of Probe

Using the CV measurement method described above, measurements of hybridization of probes 1 to 4 with respect to the following target sequence and non-target sequences were performed.

(SEQ ID NO: 973)
Target sequence: AGCUACAUUGUCUGCUGGGUUUC
(SEQ ID NO: 974)
Non-target sequence: UGGCAGGGAGGCUGGGAGGGG
(Probe)
(SEQ ID NO: 975)
Probe 23-mer: GAAACCCAGCGACAATGTAGCT-Linker
(SEQ ID NO: 976)
Probe 18-mer: CCAGCAGACAATGTAGCT-Linker
(SEQ ID NO: 977)
Probe 17-mer: CAGCAGACAATGTAGCT-Linker

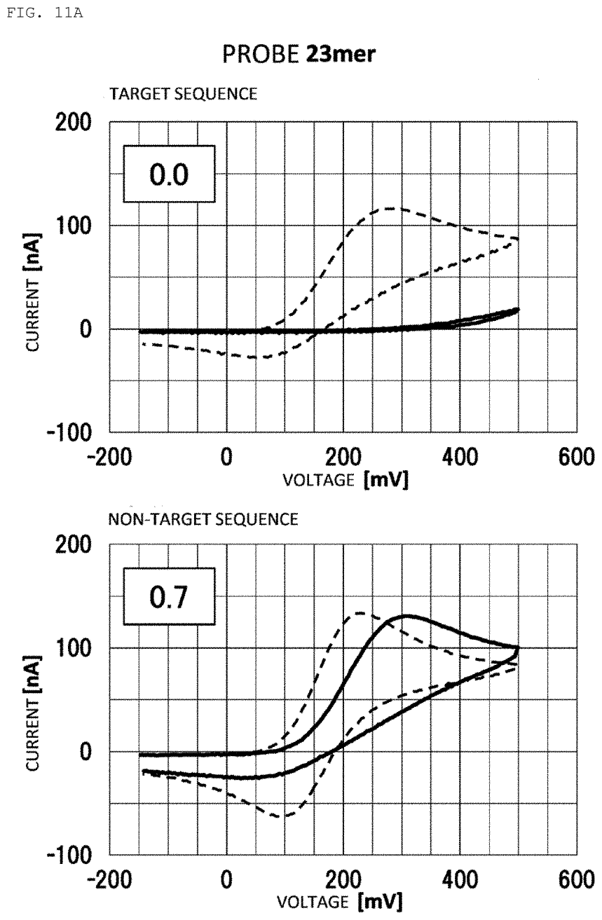

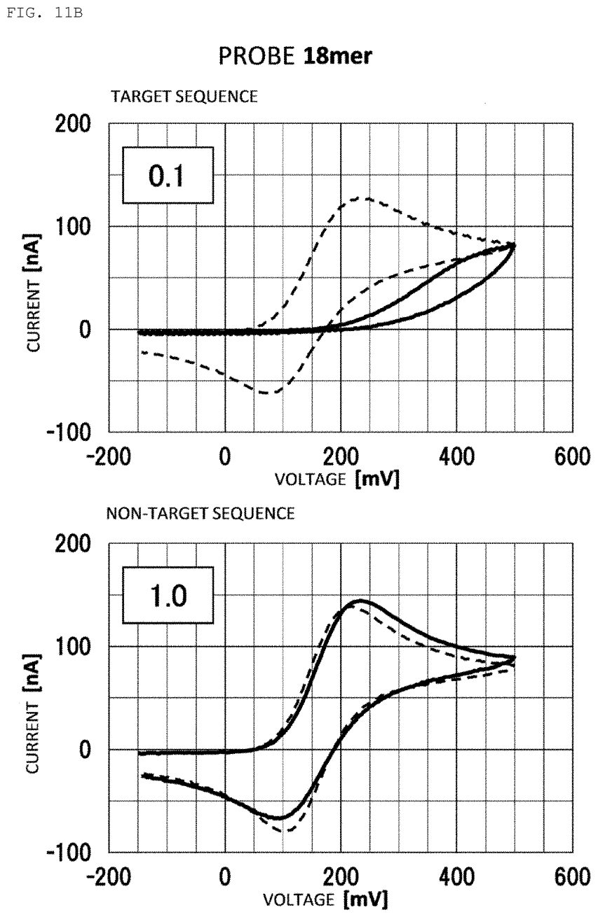

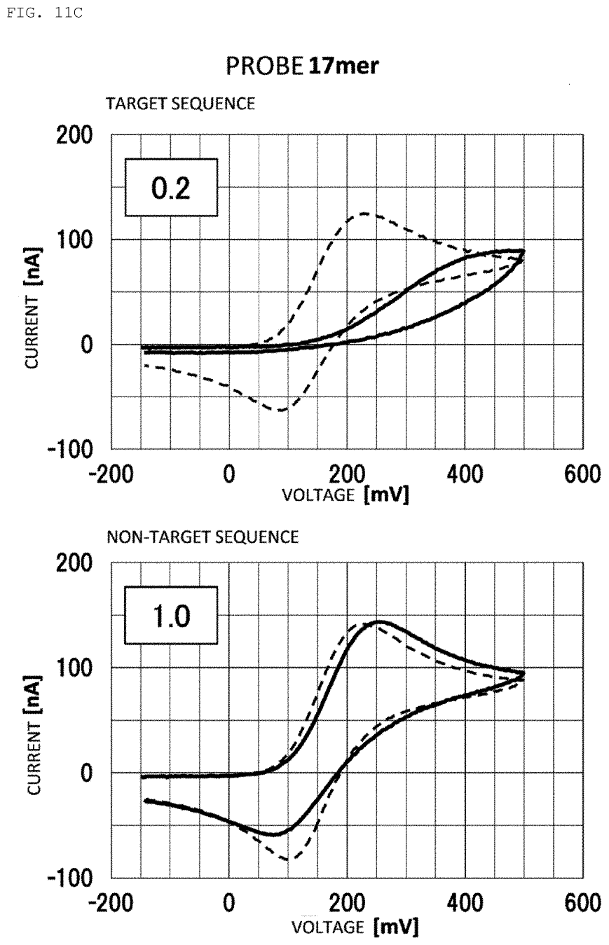

FIG. 11 shows a CV waveform when each of target sequences and non-target sequences is modified for each of probes. The CV waveform of measurement 1 is shown by a dotted line, and the CV waveform of measurement 2 is shown by a solid line, and the current value ratio is described at the upper left.

Target sequence and probe 23-mer: A current value ratio decreased to 0.0, and hybridization occurred correctly with the target sequence.

Target sequence and probe 18-mer and probe 17-mer: As the chain length decreases, the current value ratio increases, but the ratio is suppressed to 0.2. This means that the hybridization with the target sequence is reduced as compared with the full length, but the current value ratio is sufficiently reduced for the complementation determination.

Non-target sequence and probe 23-mer: A current value ratio is 0.7, and mishybridization with the non-target sequence was caused.

Non-target sequence and probe 18-mer and probe 17-mer: A current value ratio was 1.0, and mishybridization was completely suppressed. The effect obtained by cleaving the GC contiguous sequence so that the GC contiguous sequence has less than 3 consecutive bases is observed.

### SEQUENCE LISTING

TN17G016PC ST25.txt

1. A polynucleobase probe comprising:
in a sequence complementary to a target sequence having at least one sequence of any one of SEQ ID NOs: 1 to 10,
a sequence in which at least one of bases in a portion complementary to any one sequence of SEQ ID NOs: 1 to 10 in the target sequence becomes abasic and/or is substituted; and/or
a sequence which is cleaved to have, on an end, at least one sequence complementary to a sequence of 2 bases or less in any one sequence of SEQ ID NOs: 1 to 10 in the target sequence.

2. The polynucleobase probe according to claim 1, which is 10- to 50-mer.

3. The polynucleobase probe according to claim 2, which is 15- to 28-mer.

4. The polynucleobase probe according to claim 1, to which a label is bound.

5. The polynucleobase probe according to claim 1,
wherein, in the portion complementary to any one sequence of SEQ ID NOs: 1 to 10 in the target sequence, at least one of the bases which become abasic or are substituted is located inside the portion complementary to any one sequence of SEQ ID NOs: 1 to 10 in the target sequence.

6. The polynucleobase probe according to claim 1,
wherein, in the sequence complementary to any one sequence of SEQ ID NOs: I to 10 in the target sequence, a ratio of at least one of the bases which become abasic or are substituted with respect to any one of 3 to 5 guanines and cytosines is 1.

7. The polynucleobase probe according to claim 1,
wherein the target nucleic acid is a 10- to 50 mer DNA or RNA.

8. The polynucleobase probe according to claim 7,
wherein the target nucleic acid is a miRNA.

9. The polynucleobase probe according to claim 1, which is a DNA, RNA, LNA, GNA, BNA, or PNA.

10. A method for designing a polynucleobase probe sequence which is capable of binding to a target sequence having at least one sequence of any one of SEQ ID NOs: 1 to 10 with high specificity, the method comprising:
A) selecting a 10- to 50-mer sequence fully complementary to the target sequence as a. fully complementary probe sequence; and
B) (i) in the fully complementary probe sequence, designing the polynucleobase probe sequence by substituting and/or making at least one of bases abasic in a portion complementary to any one sequence of SEQ ID NOs: 1 to 10 in the target sequence, and/or
in the fully complementary probe sequence, designing the polynucleobase probe sequence by cleaving an end of the fully complementary probe sequence such that a portion complementary to any one sequence of SEQ ID NOs: 1 to 10 in the target sequence becomes 2 bases or less.

11. A method for detecting or quantitatively determining a target nucleic acid having at least one sequence of any one of SEQ ID NOs: 1 to 10 in a test sample with high specificity, the method comprising:
preparing the test sample to detect the target nucleic acid;
bringing at least one kind of the polynucleobase probes according to claim 1 in contact with the test sample; and
detecting or quantitatively determining the target nucleic acid bound to the polynucleobase probe.

12. The polynucleobase probe according to claim 3,
wherein, in the portion complementary to any one sequence of SEQ ID NOs: 1 to 10 in the target sequence, at least one of the bases which become abasic or are substituted is located inside the portion complementary to any one sequence of SEQ ID NOs: 1 to 10 in the target sequence.

13. The polynucleobase probe according to claim 4,
wherein, in the portion complementary to any one sequence of SEQ ID NOs: 1 to 10 in the target sequence, at least one of the bases which become abasic or are substituted is located inside the portion complementary to any one sequence of SEQ ID NOs: 1 to 10 in the target sequence.

14. The polynucleobase probe according to claim 3,
wherein, in the sequence complementary to any one sequence of SEQ ID NOs: 1 to 10 in the target sequence, a ratio of at least one of the bases which become abasic or are substituted with respect to any one of 3 to 5 guanines and cytosines is 1.

15. The polynucleobase probe according⁻ to claim 4,
wherein, in the sequence complementary to any one sequence of SEQ ID NOs: 1 to 10 in the target sequence, a ratio of at least one of the bases which become abasic or are substituted with respect to any one of 3 to 5 guanines and cytosines is 1.

16. The polynucleobase probe according to claim 5,
wherein, in the sequence complementary to any one sequence of SEQ ID NOs: 1 to 10 in the target sequence, a ratio of at least one of the bases which become abasic or are substituted with respect to any one of 3 to 5 guanines and cytosines is 1.

17. The polynucleobase probe according to claim 2,
wherein the target nucleic acid is a 10- to 50mer DNA or RNA.

18. The polynucleobase probe according to claim 17,
wherein the target nucleic acid is a miRNA.

19. The polynucleobase probe according to claim 5 which is a DNA, RNA, LNA, GNA, BNA, or PNA.

20. The polynucleobase probe according to claim 6, which is a DNA, RNA, LNA, GNA, BNA, or PNA.


SEQUENCE LISTING (977 sequences)

SEQ ID NO: 1 - DNA, 7 bp, Artificial Sequence
  GC continued sequence
ggggggg

SEQ ID NO: 2 - DNA, 7 bp, Artificial Sequence
  GC continued sequence
ccccccc

SEQ ID NO: 3 - DNA, 6 bp, Artificial Sequence
  GC continued sequence
gggggg

SEQ ID NO: 4 - DNA, 6 bp, Artificial Sequence
  GC continued sequence
cccccc

SEQ ID NO: 5 - DNA, 5 bp, Artificial Sequence
  GC continued sequence
ggggg

SEQ ID NO: 6 - DNA, 5 bp, Artificial Sequence
  GC continued sequence
ccccc

SEQ ID NO: 7 - DNA, 4 bp, Artificial Sequence
  GC continued sequence
gggg

SEQ ID NO: 8 - DNA, 4 bp, Artificial Sequence
  GC continued sequence
cccc

SEQ ID NO: 9 - DNA, 3 bp, Artificial Sequence
  GC continued sequence
ggg

SEQ ID NO: 10 - DNA, 3 bp, Artificial Sequence
  GC continued sequence
ccc

SEQ ID NO: 11 - DNA, 7 bp, Artificial Sequence
  Substituted/abasic GC continuous sequence
  misc_feature (2)..(2): “n” is abasic or substituted base
  misc_feature (5)..(5): “n” is abasic or substituted base
gnggngg

SEQ ID NO: 12 - DNA, 7 bp, Artificial Sequence
  Substituted/abasic GC continuous sequence
  misc_feature (3)..(3): “n” is abasic or substituted base
  misc_feature (5)..(5): “n” is abasic or substituted base
ggngngg

SEQ ID NO: 13 - DNA, 7 bp, Artificial Sequence
  Substituted/abasic GC continuous sequence
  misc_feature (3)..(3): “n” is abasic or substituted base
  misc_feature (6)..(6): “n” is abasic or substituted base
ggnggng

SEQ ID NO: 14 - DNA, 7 bp, Artificial Sequence
  Substituted/abasic GC continuous sequence
  misc_feature (1)..(1): “n” is abasic or substituted base
  misc_feature (3)..(3): “n” is abasic or substituted base
  misc_feature (5)..(5): “n” is abasic or substituted base
ngngngg

SEQ ID NO: 15 - DNA, 7 bp, Artificial Sequence
  Substituted/abasic GC continuous sequence
  misc_feature (1)..(1): “n” is abasic or substituted base
  misc_feature (3)..(3): “n” is abasic or substituted base
  misc_feature (6)..(6): “n” is abasic or substituted base
ngnggng

SEQ ID NO: 16 - DNA, 7 bp, Artificial Sequence
  Substituted/abasic GC continuous sequence
  misc_feature (1)..(1): “n” is abasic or substituted base
  misc_feature (4)..(4): “n” is abasic or substituted base
  misc_feature (6)..(6): “n” is abasic or substituted base
nggngng

SEQ ID NO: 17 - DNA, 7 bp, Artificial Sequence
  Substituted/abasic GC continuous sequence
  misc_feature (1)..(1): “n” is abasic or substituted base
  misc_feature (4)..(4): “n” is abasic or substituted base
  misc_feature (7)..(7): “n” is abasic or substituted base
nggnggn

SEQ ID NO: 18 - DNA, 7 bp, Artificial Sequence
  Substituted/abasic GC continuous sequence
  misc_feature (2)..(2): “n” is abasic or substituted base
  misc_feature (4)..(4): “n” is abasic or substituted base
  misc_feature (6)..(6): “n” is abasic or substituted base
gngngng

SEQ ID NO: 19 - DNA, 7 bp, Artificial Sequence
  Substituted/abasic GC continuous sequence
  misc_feature (2)..(2): “n” is abasic or substituted base
  misc_feature (5)..(5): “n” is abasic or substituted base
  misc_feature (7)..(7): “n” is abasic or substituted base
gnggngn

SEQ ID NO: 20 - DNA, 7 bp, Artificial Sequence
  Substituted/abasic GC continuous sequence
  misc_feature (2)..(2): “n” is abasic or substituted base
  misc_feature (4)..(4): “n” is abasic or substituted base
  misc_feature (7)..(7): “n” is abasic or substituted base
gngnggn

SEQ ID NO: 21 - DNA, 7 bp, Artificial Sequence
  Substituted/abasic GC continuous sequence
  misc_feature (3)..(3): “n” is abasic or substituted base
  misc_feature (5)..(5): “n” is abasic or substituted base
  misc_feature (7)..(7): “n” is abasic or substituted base
ggngngn

SEQ ID NO: 22 - DNA, 7 bp, Artificial Sequence
  Substituted/abasic GC continuous sequence
  misc_feature (2)..(2): “n” is abasic or substituted base
  misc_feature (5)..(5): “n” is abasic or substituted base
cnccncc

SEQ ID NO: 23 - DNA, 7 bp, Artificial Sequence
  Substituted/abasic GC continuous sequence
  misc_feature (3)..(3): “n” is abasic or substituted base
  misc_feature (5)..(5): “n” is abasic or substituted base
ccncncc

SEQ ID NO: 24 - DNA, 7 bp, Artificial Sequence
  Substituted/abasic GC continuous sequence
  misc_feature (3)..(3): “n” is abasic or substituted base
  misc_feature (6)..(6): “n” is abasic or substituted base
ccnccnc

SEQ ID NO: 25 - DNA, 7 bp, Artificial Sequence
  Substituted/abasic GC continuous sequence
  misc_feature (1)..(1): “n” is abasic or substituted base
  misc_feature (3)..(3): “n” is abasic or substituted base
  misc_feature (5)..(5): “n” is abasic or substituted base
ncncncc

SEQ ID NO: 26 - DNA, 7 bp, Artificial Sequence
  Substituted/abasic GC continuous sequence
  misc_feature (1)..(1): “n” is abasic or substituted base
  misc_feature (3)..(3): “n” is abasic or substituted base
  misc_feature (6)..(6): “n” is abasic or substituted base
ncnccnc

SEQ ID NO: 27 - DNA, 7 bp, Artificial Sequence
  Substituted/abasic GC continuous sequence
  misc_feature (1)..(1): “n” is abasic or substituted base
  misc_feature (4)..(4): “n” is abasic or substituted base
  misc_feature (6)..(6): “n” is abasic or substituted base
nccncnc

SEQ ID NO: 28 - DNA, 7 bp, Artificial Sequence
  Substituted/abasic GC continuous sequence
  misc_feature (1)..(1): “n” is abasic or substituted base
  misc_feature (4)..(4): “n” is abasic or substituted base
  misc_feature (7)..(7): “n” is abasic or substituted base
nccnccn

SEQ ID NO: 29 - DNA, 7 bp, Artificial Sequence
  Substituted/abasic GC continuous sequence
  misc_feature (2)..(2): “n” is abasic or substituted base
  misc_feature (4)..(4): “n” is abasic or substituted base
  misc_feature (6)..(6): “n” is abasic or substituted base
cncncnc

SEQ ID NO: 30 - DNA, 7 bp, Artificial Sequence
  Substituted/abasic GC continuous sequence
  misc_feature (2)..(2): “n” is abasic or substituted base
  misc_feature (4)..(4): “n” is abasic or substituted base
  misc_feature (7)..(7): “n” is abasic or substituted base
cncnccn

SEQ ID NO: 31 - DNA, 7 bp, Artificial Sequence
  Substituted/abasic GC continuous sequence
  misc_feature (2)..(2): “n” is abasic or substituted base
  misc_feature (5)..(5): “n” is abasic or substituted base
  misc_feature (7)..(7): “n” is abasic or substituted base
cnccncn

SEQ ID NO: 32 - DNA, 7 bp, Artificial Sequence
  Substituted/abasic GC continuous sequence
  misc_feature (3)..(3): “n” is abasic or substituted base
  misc_feature (5)..(5): “n” is abasic or substituted base
  misc_feature (7)..(7): “n” is abasic or substituted base
ccncncn

SEQ ID NO: 33 - DNA, 6 bp, Artificial Sequence
  Substituted/abasic GC continuous sequence
  misc_feature (1)..(1): “n” is abasic or substituted base
  misc_feature (4)..(4): “n” is abasic or substituted base
nggngg

SEQ ID NO: 34 - DNA, 6 bp, Artificial Sequence
  Substituted/abasic GC continuous sequence
  misc_feature (2)..(2): “n” is abasic or substituted base
  misc_feature (4)..(4): “n” is abasic or substituted base
gngngg

SEQ ID NO: 35 - DNA, 6 bp, Artificial Sequence
  Substituted/abasic GC continuous sequence
  misc_feature (2)..(2): “n” is abasic or substituted base
  misc_feature (5)..(5): “n” is abasic or substituted base
gnggng

SEQ ID NO: 36 - DNA, 6 bp, Artificial Sequence
  Substituted/abasic GC continuous sequence
  misc_feature (3)..(3): “n” is abasic or substituted base
  misc_feature (5)..(5): “n” is abasic or substituted base
ggngng

SEQ ID NO: 37 - DNA, 6 bp, Artificial Sequence
  Substituted/abasic GC continuous sequence
  misc_feature (3)..(3): “n” is abasic or substituted base
  misc_feature (6)..(6): “n” is abasic or substituted base
ggnggn

SEQ ID NO: 38 - DNA, 6 bp, Artificial Sequence
  Substituted/abasic GC continuous sequence
  misc_feature (1)..(1): “n” is abasic or substituted base
  misc_feature (3)..(3): “n” is abasic or substituted base
  misc_feature (5)..(5): “n” is abasic or substituted base
ngngng

SEQ ID NO: 39 - DNA, 6 bp, Artificial Sequence
  Substituted/abasic GC continuous sequence
  misc_feature (1)..(1): “n” is abasic or substituted base
  misc_feature (3)..(3): “n” is abasic or substituted base
  misc_feature (6)..(6): “n” is abasic or substituted base
ngnggn

SEQ ID NO: 40 - DNA, 6 bp, Artificial Sequence
  Substituted/abasic GC continuous sequence
  misc_feature (1)..(1): “n” is abasic or substituted base
  misc_feature (4)..(4): “n” is abasic or substituted base
  misc_feature (6)..(6): “n” is abasic or substituted base
nggngn

SEQ ID NO: 41 - DNA, 6 bp, Artificial Sequence
  Substituted/abasic GC continuous sequence
  misc_feature (2)..(2): “n” is abasic or substituted base
  misc_feature (4)..(4): “n” is abasic or substituted base
  misc_feature (6)..(6): “n” is abasic or substituted base
gngngn

SEQ ID NO: 42 - DNA, 6 bp, Artificial Sequence
  Substituted/abasic GC continuous sequence
  misc_feature (1)..(1): “n” is abasic or substituted base
  misc_feature (4)..(4): “n” is abasic or substituted base
nccncc

SEQ ID NO: 43 - DNA, 6 bp, Artificial Sequence
  Substituted/abasic GC continuous sequence
  misc_feature (2)..(2): “n” is abasic or substituted base
  misc_feature (4)..(4): “n” is abasic or substituted base
cncncc

SEQ ID NO: 44 - DNA, 6 bp, Artificial Sequence
  Substituted/abasic GC continuous sequence
  misc_feature (2)..(2): “n” is abasic or substituted base
  misc_feature (5)..(5): “n” is abasic or substituted base
cnccnc

SEQ ID NO: 45 - DNA, 6 bp, Artificial Sequence
  Substituted/abasic GC continuous sequence
  misc_feature (3)..(3): “n” is abasic or substituted base
  misc_feature (5)..(5): “n” is abasic or substituted base
ccncnc

SEQ ID NO: 46 - DNA, 6 bp, Artificial Sequence
  Substituted/abasic GC continuous sequence
  misc_feature (3)..(3): “n” is abasic or substituted base
  misc_feature (6)..(6): “n” is abasic or substituted base
ccnccn

SEQ ID NO: 47 - DNA, 6 bp, Artificial Sequence
  Substituted/abasic GC continuous sequence
  misc_feature (1)..(1): “n” is abasic or substituted base
  misc_feature (3)..(3): “n” is abasic or substituted base
  misc_feature (5)..(5): “n” is abasic or substituted base
ncncnc

SEQ ID NO: 48 - DNA, 6 bp, Artificial Sequence
  Substituted/abasic GC continuous sequence
  misc_feature (1)..(1): “n” is abasic or substituted base
  misc_feature (3)..(3): “n” is abasic or substituted base
  misc_feature (6)..(6): “n” is abasic or substituted base
ncnccn

SEQ ID NO: 49 - DNA, 6 bp, Artificial Sequence
  Substituted/abasic GC continuous sequence
  misc_feature (1)..(1): “n” is abasic or substituted base
  misc_feature (4)..(4): “n” is abasic or substituted base
  misc_feature (6)..(6): “n” is abasic or substituted base
nccncn

SEQ ID NO: 50 - DNA, 6 bp, Artificial Sequence
  Substituted/abasic GC continuous sequence
  misc_feature (2)..(2): “n” is abasic or substituted base
  misc_feature (4)..(4): “n” is abasic or substituted base
  misc_feature (6)..(6): “n” is abasic or substituted base
cncncn

SEQ ID NO: 51 - DNA, 5 bp, Artificial Sequence
  Substituted/abasic GC continuous sequence
  misc_feature (3)..(3): “n” is abasic or substituted base
ggngg

SEQ ID NO: 52 - DNA, 5 bp, Artificial Sequence
  Substituted/abasic GC continuous sequence
  misc_feature (1)..(1): “n” is abasic or substituted base
  misc_feature (3)..(3): “n” is abasic or substituted base
ngngg

SEQ ID NO: 53 - DNA, 5 bp, Artificial Sequence
  Substituted/abasic GC continuous sequence
  misc_feature (1)..(1): “n” is abasic or substituted base
  misc_feature (4)..(4): “n” is abasic or substituted base
nggng

SEQ ID NO: 54 - DNA, 5 bp, Artificial Sequence
  Substituted/abasic GC continuous sequence
  misc_feature (2)..(2): “n” is abasic or substituted base
  misc_feature (4)..(4): “n” is abasic or substituted base
gngng

SEQ ID NO: 55 - DNA, 5 bp, Artificial Sequence
  Substituted/abasic GC continuous sequence
  misc_feature (2)..(2): “n” is abasic or substituted base
  misc_feature (5)..(5): “n” is abasic or substituted base
gnggn

SEQ ID NO: 56 - DNA, 5 bp, Artificial Sequence
  Substituted/abasic GC continuous sequence
  misc_feature (3)..(3): “n” is abasic or substituted base
  misc_feature (5)..(5): “n” is abasic or substituted base
ggngn

SEQ ID NO: 57 - DNA, 5 bp, Artificial Sequence
  Substituted/abasic GC continuous sequence
  misc_feature (3)..(3): “n” is abasic or substituted base
ccncc

SEQ ID NO: 58 - DNA, 5 bp, Artificial Sequence
  Substituted/abasic GC continuous sequence
  misc_feature (1)..(1): “n” is abasic or substituted base
  misc_feature (3)..(3): “n” is abasic or substituted base
ncncc

SEQ ID NO: 59 - DNA, 5 bp, Artificial Sequence
  Substituted/abasic GC continuous sequence
  misc_feature (1)..(1): “n” is abasic or substituted base
  misc_feature (4)..(4): “n” is abasic or substituted base
nccnc

SEQ ID NO: 60 - DNA, 5 bp, Artificial Sequence
  Substituted/abasic GC continuous sequence
  misc_feature (2)..(2): “n” is abasic or substituted base
  misc_feature (4)..(4): “n” is abasic or substituted base
cncnc

SEQ ID NO: 61 - DNA, 5 bp, Artificial Sequence
  Substituted/abasic GC continuous sequence
  misc_feature (2)..(2): “n” is abasic or substituted base
  misc_feature (5)..(5): “n” is abasic or substituted base
cnccn

SEQ ID NO: 62 - DNA, 5 bp, Artificial Sequence
  Substituted/abasic GC continuous sequence
  misc_feature (3)..(3): “n” is abasic or substituted base
  misc_feature (5)..(5): “n” is abasic or substituted base
ccncn

SEQ ID NO: 63 - DNA, 4 bp, Artificial Sequence
  Substituted/abasic GC continuous sequence
  misc_feature (2)..(2): “n” is abasic or substituted base
gngg

SEQ ID NO: 64 - DNA, 4 bp, Artificial Sequence
  Substituted/abasic GC continuous sequence
  misc_feature (3)..(3): “n” is abasic or substituted base
ggng

SEQ ID NO: 65 - DNA, 4 bp, Artificial Sequence
  Substituted/abasic GC continuous sequence
  misc_feature (1)..(1): “n” is abasic or substituted base
  misc_feature (3)..(3): “n” is abasic or substituted base
ngng

SEQ ID NO: 66 - DNA, 4 bp, Artificial Sequence
  Substituted/abasic GC continuous sequence
  misc_feature (1)..(1): “n” is abasic or substituted base
  misc_feature (4)..(4): “n” is abasic or substituted base
nggn

SEQ ID NO: 67 - DNA, 4 bp, Artificial Sequence
  Substituted/abasic GC continuous sequence
  misc_feature (2)..(2): “n” is abasic or substituted base
  misc_feature (4)..(4): “n” is abasic or substituted base
gngn

SEQ ID NO: 68 - DNA, 4 bp, Artificial Sequence
  Substituted/abasic GC continuous sequence
  misc_feature (2)..(2): “n” is abasic or substituted base
cncc

SEQ ID NO: 69 - DNA, 4 bp, Artificial Sequence
  Substituted/abasic GC continuous sequence
  misc_feature (3)..(3): “n” is abasic or substituted base
ccnc

SEQ ID NO: 70 - DNA, 4 bp, Artificial Sequence
  Substituted/abasic GC continuous sequence
  misc_feature (1)..(1): “n” is abasic or substituted base
  misc_feature (3)..(3): “n” is abasic or substituted base
ncnc

SEQ ID NO: 71 - DNA, 4 bp, Artificial Sequence
  Substituted/abasic GC continuous sequence
  misc_feature (1)..(1): “n” is abasic or substituted base
  misc_feature (4)..(4): “n” is abasic or substituted base
nccn

SEQ ID NO: 72 - DNA, 4 bp, Artificial Sequence
  Substituted/abasic GC continuous sequence
  misc_feature (2)..(2): “n” is abasic or substituted base
  misc_feature (4)..(4): “n” is abasic or substituted base
cncn

SEQ ID NO: 73 - DNA, 3 bp, Artificial Sequence
  Substituted/abasic GC continuous sequence
  misc_feature (1)..(1): “n” is abasic or substituted base
ngg

SEQ ID NO: 74 - DNA, 3 bp, Artificial Sequence
  Substituted/abasic GC continuous sequence
  misc_feature (2)..(2): “n” is abasic or substituted base
gng

SEQ ID NO: 75 - DNA, 3 bp, Artificial Sequence
  Substituted/abasic GC continuous sequence
  misc_feature (3)..(3): “n” is abasic or substituted base
ggn

SEQ ID NO: 76 - DNA, 3 bp, Artificial Sequence
  Substituted/abasic GC continuous sequence
  misc_feature (1)..(1): “n” is abasic or substituted base
  misc_feature (3)..(3): “n” is abasic or substituted base
ngn

SEQ ID NO: 77 - DNA, 3 bp, Artificial Sequence
  Substituted/abasic GC continuous sequence
  misc_feature (1)..(1): “n” is abasic or substituted base
ncc

SEQ ID NO: 78 - DNA, 3 bp, Artificial Sequence
  Substituted/abasic GC continuous sequence
  misc_feature (2)..(2): “n” is abasic or substituted base
cnc

SEQ ID NO: 79 - DNA, 3 bp, Artificial Sequence
  Substituted/abasic GC continuous sequence
  misc_feature (3)..(3): “n” is abasic or substituted base
ccn

SEQ ID NO: 80 - DNA, 3 bp, Artificial Sequence
  Substituted/abasic GC continuous sequence
  misc_feature (1)..(1): n is abasic or substituted base
  misc_feature (3)..(3): n is abasic or substituted base
ncn

SEQ ID NO: 81 - RNA, 15 nt, Homo sapiens
aggagauccuggguu

SEQ ID NO: 82 - RNA, 16 nt, Homo sapiens
cucuccucccggcuuc

SEQ ID NO: 83 - RNA, 16 nt, Homo sapiens
gcagcauucauguccc

SEQ ID NO: 84 - RNA, 16 nt, Homo sapiens
aggaaacagggaccca

SEQ ID NO: 85 - RNA, 17 nt, Homo sapiens
ucgccuccuccucuccc

SEQ ID NO: 86 - RNA, 17 nt, Homo sapiens
gggauaugaagaaaaau

SEQ ID NO: 87 - RNA, 17 nt, Homo sapiens
ccugagaaaagggccaa

SEQ ID NO: 88 - RNA, 17 nt, Homo sapiens
augugggcucaggcuca

SEQ ID NO: 89 - RNA, 17 nt, Homo sapiens
ccggcauguccagggca

SEQ ID NO: 90 - RNA, 17 nt, Homo sapiens
acauugccagggaguuu

SEQ ID NO: 91 - RNA, 17 nt, Homo sapiens
cacuguggguacaugcu

SEQ ID NO: 92 - RNA, 17 nt, Homo sapiens
ucccugagcaaagccac

SEQ ID NO: 93 - RNA, 17 nt, Homo sapiens
ccaguuuucccaggauu

SEQ ID NO: 94 - RNA, 17 nt, Homo sapiens
ugagggaggagacugca

SEQ ID NO: 95 - RNA, 17 nt, Homo sapiens
acagggaggagauugua

SEQ ID NO: 96 - RNA, 17 nt, Homo sapiens
gccggacaagagggagg

SEQ ID NO: 97 - RNA, 17 nt, Homo sapiens
uuggaggcguggguuuu

SEQ ID NO: 98 - RNA, 17 nt, Homo sapiens
aggguguguguguuuuu

SEQ ID NO: 99 - RNA, 17 nt, Homo sapiens
ggagugggcuggugguu

SEQ ID NO: 100 - RNA, 17 nt, Homo sapiens
gcugggcgaggcuggca

SEQ ID NO: 101 - RNA, 17 nt, Homo sapiens
aagacugagaggaggga

SEQ ID NO: 102 - RNA, 17 nt, Homo sapiens
guggaccuggcugggac

SEQ ID NO: 103 - RNA, 17 nt, Homo sapiens
ucccuguucgggcgcca

SEQ ID NO: 104 - RNA, 17 nt, Homo sapiens
ucccaccgcugccaccc

SEQ ID NO: 105 - RNA, 17 nt, Homo sapiens
gggagucuacagcaggg

SEQ ID NO: 106 - RNA, 17 nt, Homo sapiens
cuccgggacggcugggc

SEQ ID NO: 107 - RNA, 17 nt, Homo sapiens
cgcgccgggcccggguu

SEQ ID NO: 108 - RNA, 18 nt, Homo sapiens
ucagcuggcccucauuuc

SEQ ID NO: 109 - RNA, 18 nt, Homo sapiens
aucccaccucugccacca

SEQ ID NO: 110 - RNA, 18 nt, Homo sapiens
gucccuguucaggcgcca

SEQ ID NO: 111 - RNA, 18 nt, Homo sapiens
gcaugggugguucagugg

SEQ ID NO: 112 - RNA, 18 nt, Homo sapiens
cagggaggugaaugugau

SEQ ID NO: 113 - RNA, 18 nt, Homo sapiens
uccagugcccuccucucc

SEQ ID NO: 114 - RNA, 18 nt, Homo sapiens
ccaggcucugcaguggga

SEQ ID NO: 115 - RNA, 18 nt, Homo sapiens
ugggagcuggacuacuuc

SEQ ID NO: 116 - RNA, 18 nt, Homo sapiens
ucccuggaguuucuucuu

SEQ ID NO: 117 - RNA, 18 nt, Homo sapiens
gggauucuguagcuuccu

SEQ ID NO: 118 - RNA, 18 nt, Homo sapiens
cagcagugcgcagggcug

SEQ ID NO: 119 - RNA, 18 nt, Homo sapiens
auggucaccuccgggacu

SEQ ID NO: 120 - RNA, 18 nt, Homo sapiens
aggagaagucgggaaggu

SEQ ID NO: 121 - RNA, 18 nt, Homo sapiens
gugaaggcccggcggaga

SEQ ID NO: 122 - RNA, 18 nt, Homo sapiens
gcgugugcuugcuguggg

SEQ ID NO: 123 - RNA, 18 nt, Homo sapiens
cucugggaaaugggacag

SEQ ID NO: 124 - RNA, 18 nt, Homo sapiens
aggcugggcugggacgga

SEQ ID NO: 125 - RNA, 18 nt, Homo sapiens
cccagcaggacgggagcg

SEQ ID NO: 126 - RNA, 18 nt, Homo sapiens
gggugagggcaggugguu

SEQ ID NO: 127 - RNA, 18 nt, Homo sapiens
ggugggcuucccggaggg

SEQ ID NO: 128 - RNA, 19 nt, Homo sapiens
gugaggacucgggaggugg

SEQ ID NO: 129 - RNA, 19 nt, Homo sapiens
uggauuuuuggaucaggga

SEQ ID NO: 130 - RNA, 19 nt, Homo sapiens
acuggccugggacuaccgg

SEQ ID NO: 131 - RNA, 19 nt, Homo sapiens
agggaccugagugucuaag

SEQ ID NO: 132 - RNA, 19 nt, Homo sapiens
gcauugugcagggcuauca

SEQ ID NO: 133 - RNA, 19 nt, Homo sapiens
ccugagacccuaguuccac

SEQ ID NO: 134 - RNA, 19 nt, Homo sapiens
agagcuggcugaagggcag

SEQ ID NO: 135 - RNA, 19 nt, Homo sapiens
aggcagguuaucugggcug

SEQ ID NO: 136 - RNA, 19 nt, Homo sapiens
aagucccacuaaugccagc

SEQ ID NO: 137 - RNA, 19 nt, Homo sapiens
ugggcguaucuguaugcua

SEQ ID NO: 138 - RNA, 19 nt, Homo sapiens
ccgguuccagucccuggag

SEQ ID NO: 139 - RNA, 19 nt, Homo sapiens
ugagggaguggauuguaug

SEQ ID NO: 140 - RNA, 19 nt, Homo sapiens
ggcuggucagaugggagug

SEQ ID NO: 141 - RNA, 19 nt, Homo sapiens
gugucugcuuccuguggga

SEQ ID NO: 142 - RNA, 19 nt, Homo sapiens
aagugugcagggcacuggu

SEQ ID NO: 143 - RNA, 19 nt, Homo sapiens
aguuuucccuucaagucaa

SEQ ID NO: 144 - RNA, 19 nt, Homo sapiens
cauucucguuuccuucccu

SEQ ID NO: 145 - RNA, 19 nt, Homo sapiens
gagggcggguggaggagga

SEQ ID NO: 146 - RNA, 19 nt, Homo sapiens
aucgggcccucggcgccgg

SEQ ID NO: 147 - RNA, 19 nt, Homo sapiens
cugugggcucagcucuggg

SEQ ID NO: 148 - RNA, 19 nt, Homo sapiens
ucucccuugagggcacuuu

SEQ ID NO: 149 - RNA, 19 nt, Homo sapiens
ugcccuccuuucuucccuc

SEQ ID NO: 150 - RNA, 19 nt, Homo sapiens
ucuagugcgggcguucccg

SEQ ID NO: 151 - RNA, 19 nt, Homo sapiens
ugagggaguuggguguaua

SEQ ID NO: 152 - RNA, 19 nt, Homo sapiens
ugagggaggagguugggua

SEQ ID NO: 153 - RNA, 19 nt, Homo sapiens
ugagggagugggugggagg

SEQ ID NO: 154 - RNA, 19 nt, Homo sapiens
ugucccucugggucgccca

SEQ ID NO: 155 - RNA, 20 nt, Homo sapiens
ucugcaggguuugcuuugag

SEQ ID NO: 156 - RNA, 20 nt, Homo sapiens
gugucugggcggacagcugc

SEQ ID NO: 157 - RNA, 20 nt, Homo sapiens
uaaagagcccuguggagaca

SEQ ID NO: 158 - RNA, 20 nt, Homo sapiens
ccuccugcccuccuugcugu

SEQ ID NO: 159 - RNA, 20 nt, Homo sapiens
auauggguuuacuaguuggu

SEQ ID NO: 160 - RNA, 20 nt, Homo sapiens
aggcaugggaggucagguga

SEQ ID NO: 161 - RNA, 20 nt, Homo sapiens
gcucggacugagcagguggg

SEQ ID NO: 162 - RNA, 20 nt, Homo sapiens
ucccacuacuucacuuguga

SEQ ID NO: 163 - RNA, 20 nt, Homo sapiens
uguuccucugucucccagac

SEQ ID NO: 164 - RNA, 20 nt, Homo sapiens
aaaagcugggcugagaggcg

SEQ ID NO: 165 - RNA, 20 nt, Homo sapiens
uaacggccgcgguacccuaa

SEQ ID NO: 166 - RNA, 20 nt, Homo sapiens
aaauggguggucugaggcaa

SEQ ID NO: 167 - RNA, 20 nt, Homo sapiens
ugaggagaugcugggacuga

SEQ ID NO: 168 - RNA, 20 nt, Homo sapiens
ccauggaucuccaggugggu

SEQ ID NO: 169 - RNA, 20 nt, Homo sapiens
guccgcucggcgguggccca

SEQ ID NO: 170 - RNA, 20 nt, Homo sapiens
agaccauggguucucauugu

SEQ ID NO: 171 - RNA, 20 nt, Homo sapiens
cuuauaucagaggcuguggg

SEQ ID NO: 172 - RNA, 20 nt, Homo sapiens
aggguguuucucucaucucu

SEQ ID NO: 173 - RNA, 20 nt, Homo sapiens
aggcgcacccgaccacaugc

SEQ ID NO: 174 - RNA, 20 nt, Homo sapiens
guagaggagauggcgcaggg

SEQ ID NO: 175 - RNA, 20 nt, Homo sapiens
gaauugaucaggacauaggg

SEQ ID NO: 176 - RNA, 20 nt, Homo sapiens
ugagcccuguccucccgcag

SEQ ID NO: 177 - RNA, 20 nt, Homo sapiens
agagguauagggcaugggaa

SEQ ID NO: 178 - RNA, 20 nt, Homo sapiens
ccucugggcccuuccuccag

SEQ ID NO: 179 - RNA, 20 nt, Homo sapiens
cccugagacccuaaccuuaa

SEQ ID NO: 180 - RNA, 20 nt, Homo sapiens
ugugggacugcaaaugggag

SEQ ID NO: 181 - RNA, 20 nt, Homo sapiens
augggugauggguguggugu

SEQ ID NO: 182 - RNA, 20 nt, Homo sapiens
ggagguugggaagggcagag

SEQ ID NO: 183 - RNA, 20 nt, Homo sapiens
ucucccuucaugugcccaga

SEQ ID NO: 184 - RNA, 20 nt, Homo sapiens
cuggguugggcugggcuggg

SEQ ID NO: 185 - RNA, 21 nt, Homo sapiens
gcagagcugcagaugggauuc

SEQ ID NO: 186 - RNA, 21 nt, Homo sapiens
uugcucacuguucuucccuag

SEQ ID NO: 187 - RNA, 21 nt, Homo sapiens
ccgucgccgccacccgagccg

SEQ ID NO: 188 - RNA, 21 nt, Homo sapiens
agaggauacccuuuguauguu

SEQ ID NO: 189 - RNA, 21 nt, Homo sapiens
cccggagccaggaugcagcuc

SEQ ID NO: 190 - RNA, 21 nt, Homo sapiens
agugaaugauggguucugacc

SEQ ID NO: 191 - RNA, 21 nt, Homo sapiens
ugcaggaccaagaugagcccu

SEQ ID NO: 192 - RNA, 21 nt, Homo sapiens
uggacugcccugaucuggaga

SEQ ID NO: 193 - RNA, 21 nt, Homo sapiens
uuaggccgcagaucuggguga

SEQ ID NO: 194 - RNA, 21 nt, Homo sapiens
cuuuuugcggucugggcuugc

SEQ ID NO: 195 - RNA, 21 nt, Homo sapiens
uugggacauacuuaugcuaaa

SEQ ID NO: 196 - RNA, 21 nt, Homo sapiens
uugggacauacuuaugcuaaa

SEQ ID NO: 197 - RNA, 21 nt, Homo sapiens
uugggacauacuuaugcuaaa

SEQ ID NO: 198 - RNA, 21 nt, Homo sapiens
uugggacauacuuaugcuaaa

SEQ ID NO: 199 - RNA, 21 nt, Homo sapiens
uugggacauacuuaugcuaaa

SEQ ID NO: 200 - RNA, 21 nt, Homo sapiens
acucuuucccuguugcacuac

SEQ ID NO: 201 - RNA, 21 nt, Homo sapiens
uaccacaggguagaaccacgg

SEQ ID NO: 202 - RNA, 21 nt, Homo sapiens
ugagugccggugccugcccug

SEQ ID NO: 203 - RNA, 21 nt, Homo sapiens
ugauauguuugauauuggguu

SEQ ID NO: 204 - RNA, 21 nt, Homo sapiens
caacaccagucgaugggcugu

SEQ ID NO: 205 - RNA, 21 nt, Homo sapiens
cgagccucaagcaagggacuu

SEQ ID NO: 206 - RNA, 21 nt, Homo sapiens
agcuacaucuggcuacugggu

SEQ ID NO: 207 - RNA, 21 nt, Homo sapiens
ugacagcgcccugccuggcuc

SEQ ID NO: 208 - RNA, 21 nt, Homo sapiens
aucacauugccagggauuucc

SEQ ID NO: 209 - RNA, 21 nt, Homo sapiens
aucacauugccagggauuacc

SEQ ID NO: 210 - RNA, 21 nt, Homo sapiens
aggcggagacuugggcaauug

SEQ ID NO: 211 - RNA, 21 nt, Homo sapiens
guuagggccaacaucucuugg

SEQ ID NO: 212 - RNA, 21 nt, Homo sapiens
uucgcgggcgaaggcaaaguc

SEQ ID NO: 213 - RNA, 21 nt, Homo sapiens
gcugcaccggagacuggguaa

SEQ ID NO: 214 - RNA, 21 nt, Homo sapiens
uacccagucuccggugcagcc

SEQ ID NO: 215 - RNA, 21 nt, Homo sapiens
ccaggcucugcagugggaacu

SEQ ID NO: 216 - RNA, 21 nt, Homo sapiens
cucccacuuccagaucuuucu

SEQ ID NO: 217 - RNA, 21 nt, Homo sapiens
ccugcagagaggaagcccuuc

SEQ ID NO: 218 - RNA, 21 nt, Homo sapiens
ggccagccaccaggagggcug

SEQ ID NO: 219 - RNA, 21 nt, Homo sapiens
ucaccugagcucccgugccug

SEQ ID NO: 220 - RNA, 21 nt, Homo sapiens
ugugucccauuauuggugauu

SEQ ID NO: 221 - RNA, 21 nt, Homo sapiens
ugaguguugucuacgagggca

SEQ ID NO: 222 - RNA, 21 nt, Homo sapiens
acagggccgcagauggagacu

SEQ ID NO: 223 - RNA, 21 nt, Homo sapiens
gcaggcacagacagcccuggc

SEQ ID NO: 224 - RNA, 21 nt, Homo sapiens
uuagcgguggaccgcccugcg

SEQ ID NO: 225 - RNA, 21 nt, Homo sapiens
aaaagcaucaggaaguaccca

SEQ ID NO: 226 - RNA, 21 nt, Homo sapiens
gaccgagagggccucggcugu

SEQ ID NO: 227 - RNA, 21 nt, Homo sapiens
auuggacugcugauggcccgu

SEQ ID NO: 228 - RNA, 21 nt, Homo sapiens
gcaguccaugggcauauacac

SEQ ID NO: 229 - RNA, 21 nt, Homo sapiens
ucuugaagucagaacccgcaa

SEQ ID NO: 230 - RNA, 21 nt, Homo sapiens
gcagcccagcugaggccucug

SEQ ID NO: 231 - RNA, 21 nt, Homo sapiens
acacauggguggcuguggccu

SEQ ID NO: 232 - RNA, 21 nt, Homo sapiens
gcccugaccuguccuguucug

SEQ ID NO: 233 - RNA, 21 nt, Homo sapiens
agcggugcuccugcgggccga

SEQ ID NO: 234 - RNA, 21 nt, Homo sapiens
gagggcaugcgcacuuugucc

SEQ ID NO: 235 - RNA, 21 nt, Homo sapiens
ugcuuaaccuugcccucgaaa

SEQ ID NO: 236 - RNA, 21 nt, Homo sapiens
ucacuccucuccucccgucuu

SEQ ID NO: 237 - RNA, 21 nt, Homo sapiens
cccagauaauggcacucucaa

SEQ ID NO: 238 - RNA, 21 nt, Homo sapiens
uuucccuuuccauccuggcag

SEQ ID NO: 239 - RNA, 21 nt, Homo sapiens
uugccagggcaggagguggaa

SEQ ID NO: 240 - RNA, 21 nt, Homo sapiens
auccuugcuaucugggugcua

SEQ ID NO: 241 - RNA, 21 nt, Homo sapiens
uaaggcacccuucugaguaga

SEQ ID NO: 242 - RNA, 21 nt, Homo sapiens
gugggauuucugaguagcauc

SEQ ID NO: 243 - RNA, 21 nt, Homo sapiens
caauucucaaaggagccuccc

SEQ ID NO: 244 - RNA, 21 nt, Homo sapiens
acuggcauuagugggacuuuu

SEQ ID NO: 245 - RNA, 21 nt, Homo sapiens
ggcugggugcucuugugcagu

SEQ ID NO: 246 - RNA, 21 nt, Homo sapiens
cagaucaugggacugucucag

SEQ ID NO: 247 - RNA, 21 nt, Homo sapiens
caaagaggaaggucccauuac

SEQ ID NO: 248 - RNA, 21 nt, Homo sapiens
uuggccacaauggguuagaac

SEQ ID NO: 249 - RNA, 21 nt, Homo sapiens
acggcccaggcggcauuggug

SEQ ID NO: 250 - RNA, 21 nt, Homo sapiens
gggaagagcuguacggccuuc

SEQ ID NO: 251 - RNA, 21 nt, Homo sapiens
ugagcuaaaugugugcuggga

SEQ ID NO: 252 - RNA, 21 nt, Homo sapiens
agaccuggcccagaccucagc

SEQ ID NO: 253 - RNA, 21 nt, Homo sapiens
acacuuguugggaugaccugc

SEQ ID NO: 254 - RNA, 21 nt, Homo sapiens
gggacuaggaugcagaccucc

SEQ ID NO: 255 - RNA, 21 nt, Homo sapiens
gaagaauaggagggacuuugu

SEQ ID NO: 256 - RNA, 21 nt, Homo sapiens
ucuagaaaugcaugacccacc

SEQ ID NO: 257 - RNA, 21 nt, Homo sapiens
ucaagugucaucugucccuag

SEQ ID NO: 258 - RNA, 21 nt, Homo sapiens
uuggaggguguggaagacauc

SEQ ID NO: 259 - RNA, 21 nt, Homo sapiens
aauggcgccacuaggguugug

SEQ ID NO: 260 - RNA, 21 nt, Homo sapiens
uccgguucucagggcuccacc

SEQ ID NO: 261 - RNA, 21 nt, Homo sapiens
uaguggucagagggcuuauga

SEQ ID NO: 262 - RNA, 21 nt, Homo sapiens
cacgcgggaaccgaguccacc

SEQ ID NO: 263 - RNA, 21 nt, Homo sapiens
cgacgagggccggucggucgc

SEQ ID NO: 264 - RNA, 21 nt, Homo sapiens
ucacccuuccauaucuagucu

SEQ ID NO: 265 - RNA, 21 nt, Homo sapiens
uccugcgcgucccagaugccc

SEQ ID NO: 266 - RNA, 21 nt, Homo sapiens
cucccacaugcaggguuugca

SEQ ID NO: 267 - RNA, 21 nt, Homo sapiens
caucccuugcaugguggaggg

SEQ ID NO: 268 - RNA, 21 nt, Homo sapiens
ccucccaugccaagaacuccc

SEQ ID NO: 269 - RNA, 21 nt, Homo sapiens
ugggcucaggguacaaagguu

SEQ ID NO: 270 - RNA, 21 nt, Homo sapiens
cugggcucgggacgcgcggcu

SEQ ID NO: 271 - RNA, 21 nt, Homo sapiens
agacccugcagccuucccacc

SEQ ID NO: 272 - RNA, 21 nt, Homo sapiens
cgccugcccagcccuccugcu

SEQ ID NO: 273 - RNA, 21 nt, Homo sapiens
ccugugcucccagggccucgc

SEQ ID NO: 274 - RNA, 21 nt, Homo sapiens
ucauccucgucucccucccag

SEQ ID NO: 275 - RNA, 21 nt, Homo sapiens
auacccauagcuuagcuccca

SEQ ID NO: 276 - RNA, 21 nt, Homo sapiens
ugggagcuaagcuauggguau

SEQ ID NO: 277 - RNA, 21 nt, Homo sapiens
aagccugcccggcuccucggg

SEQ ID NO: 278 - RNA, 21 nt, Homo sapiens
uggguuuacguugggagaacu

SEQ ID NO: 279 - RNA, 21 nt, Homo sapiens
ucccacguuguggcccagcag

SEQ ID NO: 280 - RNA, 21 nt, Homo sapiens
cgcgggugcuuacugacccuu

SEQ ID NO: 281 - RNA, 21 nt, Homo sapiens
cuccuucacccgggcgguacc

SEQ ID NO: 282 - RNA, 21 nt, Homo sapiens
cuuccgcccggccgggugucg

SEQ ID NO: 283 - RNA, 21 nt, Homo sapiens
auugucccucucccuucccag

SEQ ID NO: 284 - RNA, 21 nt, Homo sapiens
ggcgcgcccagcucccgggcu

SEQ ID NO: 285 - RNA, 22 nt, Homo sapiens
cuauacaaccuacugccuuccc

SEQ ID NO: 286 - RNA, 22 nt, Homo sapiens
cuauacaaucuauugccuuccc

SEQ ID NO: 287 - RNA, 22 nt, Homo sapiens
aacccguagauccgaacuugug

SEQ ID NO: 288 - RNA, 22 nt, Homo sapiens
ccgcacuguggguacuugcugc

SEQ ID NO: 289 - RNA, 22 nt, Homo sapiens
uuuccggcucgcgugggugugu

SEQ ID NO: 290 - RNA, 22 nt, Homo sapiens
acaggugagguucuugggagcc

SEQ ID NO: 291 - RNA, 22 nt, Homo sapiens
ucacaagucaggcucuugggac

SEQ ID NO: 292 - RNA, 22 nt, Homo sapiens
augggugaauuuguagaaggau

SEQ ID NO: 293 - RNA, 22 nt, Homo sapiens
augguacccuggcauacugagu

SEQ ID NO: 294 - RNA, 22 nt, Homo sapiens
cugaagcucagagggcucugau

SEQ ID NO: 295 - RNA, 22 nt, Homo sapiens
ucuauacagacccuggcuuuuc

SEQ ID NO: 296 - RNA, 22 nt, Homo sapiens
ucugggcaacaaagugagaccu

SEQ ID NO: 297 - RNA, 22 nt, Homo sapiens
uggguggucuggagauuugugc

SEQ ID NO: 298 - RNA, 22 nt, Homo sapiens
uucuggaauucugugugaggga

SEQ ID NO: 299 - RNA, 22 nt, Homo sapiens
uuuucaacucuaaugggagaga

SEQ ID NO: 300 - RNA, 22 nt, Homo sapiens
cagugcaauguuaaaagggcau

SEQ ID NO: 301 - RNA, 22 nt, Homo sapiens
cagugcaaugaugaaagggcau

SEQ ID NO: 302 - RNA, 22 nt, Homo sapiens
uauagggauuggagccguggcg

SEQ ID NO: 303 - RNA, 22 nt, Homo sapiens
gcuacuucacaacaccagggcc

SEQ ID NO: 304 - RNA, 22 nt, Homo sapiens
gcuauuucacgacaccaggguu

SEQ ID NO: 305 - RNA, 22 nt, Homo sapiens
ggagacgcggcccuguuggagu

SEQ ID NO: 306 - RNA, 22 nt, Homo sapiens
cagugguuuuacccuaugguag

SEQ ID NO: 307 - RNA, 22 nt, Homo sapiens
ugagaacugaauuccauggguu

SEQ ID NO: 308 - RNA, 22 nt, Homo sapiens
ugcccuguggacucaguucugg

SEQ ID NO: 309 - RNA, 22 nt, Homo sapiens
gcccgcguguggagccaggugu

SEQ ID NO: 310 - RNA, 22 nt, Homo sapiens
gugaauuaccgaagggccauaa

SEQ ID NO: 311 - RNA, 22 nt, Homo sapiens
uggacggagaacugauaagggu

SEQ ID NO: 312 - RNA, 22 nt, Homo sapiens
caaagaauucuccuuuugggcu

SEQ ID NO: 313 - RNA, 22 nt, Homo sapiens
uacccagagcaugcagugugaa

SEQ ID NO: 314 - RNA, 22 nt, Homo sapiens
aacuggccuacaaagucccagu

SEQ ID NO: 315 - RNA, 22 nt, Homo sapiens
uagguaguuucauguuguuggg

SEQ ID NO: 316 - RNA, 22 nt, Homo sapiens
uagguaguuuccuguuguuggg

SEQ ID NO: 317 - RNA, 22 nt, Homo sapiens
uucaccaccuucuccacccagc

SEQ ID NO: 318 - RNA, 22 nt, Homo sapiens
gauuagggugcuuagcuguuaa

SEQ ID NO: 319 - RNA, 22 nt, Homo sapiens
cucccacugcuucacuugacua

SEQ ID NO: 320 - RNA, 22 nt, Homo sapiens
caucuuacugggcagcauugga

SEQ ID NO: 321 - RNA, 22 nt, Homo sapiens
cgucuuacccagcaguguuugg

SEQ ID NO: 322 - RNA, 22 nt, Homo sapiens
uucccuuugucauccuaugccu

SEQ ID NO: 323 - RNA, 22 nt, Homo sapiens
acuguaguaugggcacuuccag

SEQ ID NO: 324 - RNA, 22 nt, Homo sapiens
uucccuuugucauccuucgccu

SEQ ID NO: 325 - RNA, 22 nt, Homo sapiens
uagucccuuccuugaagcgguc

SEQ ID NO: 326 - RNA, 22 nt, Homo sapiens
agaguugagucuggacgucccg

SEQ ID NO: 327 - RNA, 22 nt, Homo sapiens
acacagggcuguugugaagacu

SEQ ID NO: 328 - RNA, 22 nt, Homo sapiens
uggguuccuggcaugcugauuu

SEQ ID NO: 329 - RNA, 22 nt, Homo sapiens
agggcuuagcugcuugugagca

SEQ ID NO: 330 - RNA, 22 nt, Homo sapiens
uaugugggaugguaaaccgcuu

SEQ ID NO: 331 - RNA, 22 nt, Homo sapiens
ugguuuaccgucccacauacau

SEQ ID NO: 332 - RNA, 22 nt, Homo sapiens
uauacaagggcagacucucucu

SEQ ID NO: 333 - RNA, 22 nt, Homo sapiens
cugggagguggauguuuacuuc

SEQ ID NO: 334 - RNA, 22 nt, Homo sapiens
cugggagaaggcuguuuacucu

SEQ ID NO: 335 - RNA, 22 nt, Homo sapiens
ugccuggaacauaguagggacu

SEQ ID NO: 336 - RNA, 22 nt, Homo sapiens
guugggacaagaggacggucuu

SEQ ID NO: 337 - RNA, 22 nt, Homo sapiens
acuuuccucacucccgugaagu

SEQ ID NO: 338 - RNA, 22 nt, Homo sapiens
ugagggacagaugccagaagca

SEQ ID NO: 339 - RNA, 22 nt, Homo sapiens
uaaagaacucuuaaaacccaau

SEQ ID NO: 340 - RNA, 22 nt, Homo sapiens
uggcccaaccuauucaguuagu

SEQ ID NO: 341 - RNA, 22 nt, Homo sapiens
agcuuuugggaauucagguagu

SEQ ID NO: 342 - RNA, 22 nt, Homo sapiens
aaagaucuggaagugggagaca

SEQ ID NO: 343 - RNA, 22 nt, Homo sapiens
cugcccuagucuagcugaagcu

SEQ ID NO: 344 - RNA, 22 nt, Homo sapiens
aagggcuuccucucugcaggac

SEQ ID NO: 345 - RNA, 22 nt, Homo sapiens
agagcugagacuagaaagccca

SEQ ID NO: 346 - RNA, 22 nt, Homo sapiens
uauaaaaugagggcaguaagac

SEQ ID NO: 347 - RNA, 22 nt, Homo sapiens
ugugacuuuaagggaaauggcg

SEQ ID NO: 348 - RNA, 22 nt, Homo sapiens
ugcccugccuguuuucuccuuu

SEQ ID NO: 349 - RNA, 22 nt, Homo sapiens
uggaagggagaagagcuuuaau

SEQ ID NO: 350 - RNA, 22 nt, Homo sapiens
ucucugggccugugucuuaggc

SEQ ID NO: 351 - RNA, 22 nt, Homo sapiens
cagugccucggcagugcagccc

SEQ ID NO: 352 - RNA, 22 nt, Homo sapiens
gcugacuccuaguccagggcuc

SEQ ID NO: 353 - RNA, 22 nt, Homo sapiens
caaucagcaaguauacugcccu

SEQ ID NO: 354 - RNA, 22 nt, Homo sapiens
agguagacugggauuuguuguu

SEQ ID NO: 355 - RNA, 22 nt, Homo sapiens
aaagacauaguugcaagauggg

SEQ ID NO: 356 - RNA, 22 nt, Homo sapiens
gggaccauccugccugcugugg

SEQ ID NO: 357 - RNA, 22 nt, Homo sapiens
ucaccugaccucccaugccugu

SEQ ID NO: 358 - RNA, 22 nt, Homo sapiens
caggcacgggagcucaggugag

SEQ ID NO: 359 - RNA, 22 nt, Homo sapiens
cggguggaucacgaugcaauuu

SEQ ID NO: 360 - RNA, 22 nt, Homo sapiens
ugaccugggacucggacagcug

SEQ ID NO: 361 - RNA, 22 nt, Homo sapiens
accuuccucuccaugggucuuu

SEQ ID NO: 362 - RNA, 22 nt, Homo sapiens
aaagacccauugaggagaaggu

SEQ ID NO: 363 - RNA, 22 nt, Homo sapiens
cucgugggcucuggccacggcc

SEQ ID NO: 364 - RNA, 22 nt, Homo sapiens
caguggccagagcccugcagug

SEQ ID NO: 365 - RNA, 22 nt, Homo sapiens
uuuccuacccuaccugaagacu

SEQ ID NO: 366 - RNA, 22 nt, Homo sapiens
cugggaggugugauaucguggu

SEQ ID NO: 367 - RNA, 22 nt, Homo sapiens
ugugauaucaugguuccuggga

SEQ ID NO: 368 - RNA, 22 nt, Homo sapiens
cugggaggugugauauuguggu

SEQ ID NO: 369 - RNA, 22 nt, Homo sapiens
ugugauaucaugguuccuggga

SEQ ID NO: 370 - RNA, 22 nt, Homo sapiens
cugggaggugugauauuguggu

SEQ ID NO: 371 - RNA, 22 nt, Homo sapiens
gggaggugugaucucacacucg

SEQ ID NO: 372 - RNA, 22 nt, Homo sapiens
ugugauaucaugguuccuggga

SEQ ID NO: 373 - RNA, 22 nt, Homo sapiens
ugugauaucgugcuuccuggga

SEQ ID NO: 374 - RNA, 22 nt, Homo sapiens
agagguugcccuuggugaauuc

SEQ ID NO: 375 - RNA, 22 nt, Homo sapiens
uauacaagggcaagcucucugu

SEQ ID NO: 376 - RNA, 22 nt, Homo sapiens
uguccucuagggccugcagucu

SEQ ID NO: 377 - RNA, 22 nt, Homo sapiens
agacaucaagaucagucccaaa

SEQ ID NO: 378 - RNA, 22 nt, Homo sapiens
uuugggacugaucuugaugucu

SEQ ID NO: 379 - RNA, 22 nt, Homo sapiens
aacuaguaauguuggauuaggg

SEQ ID NO: 380 - RNA, 22 nt, Homo sapiens
ugcucagguugcacagcuggga

SEQ ID NO: 381 - RNA, 22 nt, Homo sapiens
acuggacuuagggucagaaggc

SEQ ID NO: 382 - RNA, 22 nt, Homo sapiens
cugggacaggaggaggaggcag

SEQ ID NO: 383 - RNA, 22 nt, Homo sapiens
caggucgucuugcagggcuucu

SEQ ID NO: 384 - RNA, 22 nt, Homo sapiens
aucaugaugggcuccucggugu

SEQ ID NO: 385 - RNA, 22 nt, Homo sapiens
aguugccuuuuuguucccaugc

SEQ ID NO: 386 - RNA, 22 nt, Homo sapiens
uguugggauucagcaggaccau

SEQ ID NO: 387 - RNA, 22 nt, Homo sapiens
caaggagacgggaacauggagc

SEQ ID NO: 388 - RNA, 22 nt, Homo sapiens
cagggcaggaagaaguggacaa

SEQ ID NO: 389 - RNA, 22 nt, Homo sapiens
guccacuucugccugcccugcc

SEQ ID NO: 390 - RNA, 22 nt, Homo sapiens
uggcggcgguaguuaugggcuu

SEQ ID NO: 391 - RNA, 22 nt, Homo sapiens
ugggaacuuaguagagguuuaa

SEQ ID NO: 392 - RNA, 22 nt, Homo sapiens
caagggaccaagcauucauuau

SEQ ID NO: 393 - RNA, 22 nt, Homo sapiens
caggaaggauuuagggacaggc

SEQ ID NO: 394 - RNA, 22 nt, Homo sapiens
uugcauauguaggaugucccau

SEQ ID NO: 395 - RNA, 22 nt, Homo sapiens
ucugguaagagauuugggcaua

SEQ ID NO: 396 - RNA, 22 nt, Homo sapiens
gaggaaacugaagcugagaggg

SEQ ID NO: 397 - RNA, 22 nt, Homo sapiens
uugggaucauuuugcauccaua

SEQ ID NO: 398 - RNA, 22 nt, Homo sapiens
ugagggaguaggauguaugguu

SEQ ID NO: 399 - RNA, 22 nt, Homo sapiens
gaagaacuguugcauuugcccu

SEQ ID NO: 400 - RNA, 22 nt, Homo sapiens
agacugacggcuggaggcccau

SEQ ID NO: 401 - RNA, 22 nt, Homo sapiens
gcugacagcagggcuggccgcu

SEQ ID NO: 402 - RNA, 22 nt, Homo sapiens
gagcuuggaugagcugggcuga

SEQ ID NO: 403 - RNA, 22 nt, Homo sapiens
acccuaucaauauugucucugc

SEQ ID NO: 404 - RNA, 22 nt, Homo sapiens
ugccgcccucucgcugcucuag

SEQ ID NO: 405 - RNA, 22 nt, Homo sapiens
ugugggaucuggaggcaucugg

SEQ ID NO: 406 - RNA, 22 nt, Homo sapiens
uguguccgggaaguggaggagg

SEQ ID NO: 407 - RNA, 22 nt, Homo sapiens
aacgggaaugcaggcuguaucu

SEQ ID NO: 408 - RNA, 22 nt, Homo sapiens
gagcaggcgaggcugggcugaa

SEQ ID NO: 409 - RNA, 22 nt, Homo sapiens
ucaggcaguguggguaucagau

SEQ ID NO: 410 - RNA, 22 nt, Homo sapiens
caggaggcagugggcgagcagg

SEQ ID NO: 411 - RNA, 22 nt, Homo sapiens
uaggccacagccacccaugugu

SEQ ID NO: 412 - RNA, 22 nt, Homo sapiens
ugagggcuccaggugacggugg

SEQ ID NO: 413 - RNA, 22 nt, Homo sapiens
accugccagcaccucccugcag

SEQ ID NO: 414 - RNA, 22 nt, Homo sapiens
auagugggaagcuggcagauuc

SEQ ID NO: 415 - RNA, 22 nt, Homo sapiens
ucauuuaucuguugggaagcua

SEQ ID NO: 416 - RNA, 22 nt, Homo sapiens
ccaccaggucuagcauugggau

SEQ ID NO: 417 - RNA, 22 nt, Homo sapiens
aggacugauccucucgggcagg

SEQ ID NO: 418 - RNA, 22 nt, Homo sapiens
agggaaggaggcuuggucuuag

SEQ ID NO: 419 - RNA, 22 nt, Homo sapiens
cucgggcggaggugguugagug

SEQ ID NO: 420 - RNA, 22 nt, Homo sapiens
agccaggcucugaagggaaagu

SEQ ID NO: 421 - RNA, 22 nt, Homo sapiens
uuucccuucagagccuggcuuu

SEQ ID NO: 422 - RNA, 22 nt, Homo sapiens
uuaacuccuuucacacccaugg

SEQ ID NO: 423 - RNA, 22 nt, Homo sapiens
auucucucuggaucccauggau

SEQ ID NO: 424 - RNA, 22 nt, Homo sapiens
guggaccaggauggcaagggcu

SEQ ID NO: 425 - RNA, 22 nt, Homo sapiens
uaggagggaauaguaaaagcag

SEQ ID NO: 426 - RNA, 22 nt, Homo sapiens
ugaagccagcucuggucugggc

SEQ ID NO: 427 - RNA, 22 nt, Homo sapiens
uuacggaccagcuaagggaggc

SEQ ID NO: 428 - RNA, 22 nt, Homo sapiens
aaucauacagggacauccaguu

SEQ ID NO: 429 - RNA, 22 nt, Homo sapiens
aaucguacagggucauccacuu

SEQ ID NO: 430 - RNA, 22 nt, Homo sapiens
cuuaugcaagauucccuucuac

SEQ ID NO: 431 - RNA, 22 nt, Homo sapiens
ugaaacauacacgggaaaccuc

SEQ ID NO: 432 - RNA, 22 nt, Homo sapiens
augcaccugggcaaggauucug

SEQ ID NO: 433 - RNA, 22 nt, Homo sapiens
cuuggauuuuccugggccucag

SEQ ID NO: 434 - RNA, 22 nt, Homo sapiens
ugaggacagggcaaauucacga

SEQ ID NO: 435 - RNA, 22 nt, Homo sapiens
aaugcaccugggcaaggauuca

SEQ ID NO: 436 - RNA, 22 nt, Homo sapiens
agacccuggucugcacucuauc

SEQ ID NO: 437 - RNA, 22 nt, Homo sapiens
gggagccaggaaguauugaugu

SEQ ID NO: 438 - RNA, 22 nt, Homo sapiens
ugauugguacgucuguggguag

SEQ ID NO: 439 - RNA, 22 nt, Homo sapiens
uucagaucccagcggugccucu

SEQ ID NO: 440 - RNA, 22 nt, Homo sapiens
uccuccucuaccucaucccagu

SEQ ID NO: 441 - RNA, 22 nt, Homo sapiens
gaauauggguauauuaguuugg

SEQ ID NO: 442 - RNA, 22 nt, Homo sapiens
aaacuaauauacccauauucug

SEQ ID NO: 443 - RNA, 22 nt, Homo sapiens
cugaauagcugggacuacaggu

SEQ ID NO: 444 - RNA, 22 nt, Homo sapiens
agaaaggguggcaauaccucuu

SEQ ID NO: 445 - RNA, 22 nt, Homo sapiens
agguauugccacccuuucuagu

SEQ ID NO: 446 - RNA, 22 nt, Homo sapiens
uuagaacguuuuagggucaaau

SEQ ID NO: 447 - RNA, 22 nt, Homo sapiens
caaauaauaccacagugggugu

SEQ ID NO: 448 - RNA, 22 nt, Homo sapiens
ugagucagcaacauaucccaug

SEQ ID NO: 449 - RNA, 22 nt, Homo sapiens
uuaggccaucaucccauuaugc

SEQ ID NO: 450 - RNA, 22 nt, Homo sapiens
cacgcucaugcacacacccaca

SEQ ID NO: 451 - RNA, 22 nt, Homo sapiens
uuaugguuugccugggacugag

SEQ ID NO: 452 - RNA, 22 nt, Homo sapiens
agucauuggaggguuugagcag

SEQ ID NO: 453 - RNA, 22 nt, Homo sapiens
acucaaaacccuucagugacuu

SEQ ID NO: 454 - RNA, 22 nt, Homo sapiens
agacuucccauuugaagguggc

SEQ ID NO: 455 - RNA, 22 nt, Homo sapiens
aguauucuguaccagggaaggu

SEQ ID NO: 456 - RNA, 22 nt, Homo sapiens
gucccucuccaaaugugucuug

SEQ ID NO: 457 - RNA, 22 nt, Homo sapiens
agcaguguuuguuuugcccaca

SEQ ID NO: 458 - RNA, 22 nt, Homo sapiens
ucgggcgcaagagcacugcagu

SEQ ID NO: 459 - RNA, 22 nt, Homo sapiens
aguugccagggcugccuuuggu

SEQ ID NO: 460 - RNA, 22 nt, Homo sapiens
caaaguccuuccuauuuuuccc

SEQ ID NO: 461 - RNA, 22 nt, Homo sapiens
ugggccaugcauuucuagaacu

SEQ ID NO: 462 - RNA, 22 nt, Homo sapiens
ccucaccaucccuucugccugc

SEQ ID NO: 463 - RNA, 22 nt, Homo sapiens
uuccagcccuucuaaugguagg

SEQ ID NO: 464 - RNA, 22 nt, Homo sapiens
uggugggccgcagaacaugugc

SEQ ID NO: 465 - RNA, 22 nt, Homo sapiens
uacccauugcauaucggaguug

SEQ ID NO: 466 - RNA, 22 nt, Homo sapiens
gcacugagaugggaguggugua

SEQ ID NO: 467 - RNA, 22 nt, Homo sapiens
caacaaaucccagucuaccuaa

SEQ ID NO: 468 - RNA, 22 nt, Homo sapiens
ugagaccucuggguucugagcu

SEQ ID NO: 469 - RNA, 22 nt, Homo sapiens
uccauuacacuacccugccucu

SEQ ID NO: 470 - RNA, 22 nt, Homo sapiens
gacugacaccucuuugggugaa

SEQ ID NO: 471 - RNA, 22 nt, Homo sapiens
cacuggcuccuuucuggguaga

SEQ ID NO: 472 - RNA, 22 nt, Homo sapiens
uauugcacuugucccggccugu

SEQ ID NO: 473 - RNA, 22 nt, Homo sapiens
uauugcacucgucccggccucc

SEQ ID NO: 474 - RNA, 22 nt, Homo sapiens
acugcugagcuagcacuucccg

SEQ ID NO: 475 - RNA, 22 nt, Homo sapiens
acaguagagggaggaaucgcag

SEQ ID NO: 476 - RNA, 22 nt, Homo sapiens
uucaacggguauuuauugagca

SEQ ID NO: 477 - RNA, 22 nt, Homo sapiens
aacccguagauccgaucuugug

SEQ ID NO: 478 - RNA, 22 nt, Homo sapiens
caagcucgcuucuaugggucug

SEQ ID NO: 479 - RNA, 22 nt, Homo sapiens
cacccguagaaccgaccuugcg

SEQ ID NO: 480 - RNA, 22 nt, Homo sapiens
caagcucgugucuguggguccg

SEQ ID NO: 481 - RNA, 22 nt, Homo sapiens
caucaaaguggaggcccucucu

SEQ ID NO: 482 - RNA, 22 nt, Homo sapiens
gaucaaaguggaggcccucucc

SEQ ID NO: 483 - RNA, 22 nt, Homo sapiens
aaagugcuucccuuuugugugu

SEQ ID NO: 484 - RNA, 22 nt, Homo sapiens
uucacaaagcccauacacuuuc

SEQ ID NO: 485 - RNA, 22 nt, Homo sapiens
gaucagggccuuucuaaguaga

SEQ ID NO: 486 - RNA, 22 nt, Homo sapiens
agcgggcacagcugugagagcc

SEQ ID NO: 487 - RNA, 22 nt, Homo sapiens
aucccugaguguauguggugaa

SEQ ID NO: 488 - RNA, 22 nt, Homo sapiens
auugcuucccagacggugaaga

SEQ ID NO: 489 - RNA, 22 nt, Homo sapiens
agauugggcauaggugacugaa

SEQ ID NO: 490 - RNA, 22 nt, Homo sapiens
agagaaacccugucucaaaaaa

SEQ ID NO: 491 - RNA, 22 nt, Homo sapiens
gaaagccaccaugcuggguaaa

SEQ ID NO: 492 - RNA, 22 nt, Homo sapiens
gcagcagggugaaacugacaca

SEQ ID NO: 493 - RNA, 22 nt, Homo sapiens
ccagcugggaagaaccaguggc

SEQ ID NO: 494 - RNA, 22 nt, Homo sapiens
gcaugacaccacacuggguaga

SEQ ID NO: 495 - RNA, 22 nt, Homo sapiens
uacugagaauggguagcaguca

SEQ ID NO: 496 - RNA, 22 nt, Homo sapiens
cagcccugcugucuuaaccucu

SEQ ID NO: 497 - RNA, 22 nt, Homo sapiens
uagaguuacacccugggaguua

SEQ ID NO: 498 - RNA, 22 nt, Homo sapiens
ucaccagcccuguguucccuag

SEQ ID NO: 499 - RNA, 22 nt, Homo sapiens
ucccugagacccuaacuuguga

SEQ ID NO: 500 - RNA, 22 nt, Homo sapiens
acggguuaggcucuugggagcu

SEQ ID NO: 501 - RNA, 22 nt, Homo sapiens
uuagggcccuggcuccaucucc

SEQ ID NO: 502 - RNA, 22 nt, Homo sapiens
auguagggcuaaaagccauggg

SEQ ID NO: 503 - RNA, 22 nt, Homo sapiens
ucucccaacccuuguaccagug

SEQ ID NO: 504 - RNA, 22 nt, Homo sapiens
gcccaaaggugaauuuuuuggg

SEQ ID NO: 505 - RNA, 22 nt, Homo sapiens
ggcuacaacacaggacccgggc

SEQ ID NO: 506 - RNA, 22 nt, Homo sapiens
accuugccuugcugcccgggcc

SEQ ID NO: 507 - RNA, 22 nt, Homo sapiens
ugggucuuugcgggcgagauga

SEQ ID NO: 508 - RNA, 22 nt, Homo sapiens
aacuggcccucaaagucccgcu

SEQ ID NO: 509 - RNA, 22 nt, Homo sapiens
gaggguuggguggaggcucucc

SEQ ID NO: 510 - RNA, 22 nt, Homo sapiens
cugggagaggguuguuuacucc

SEQ ID NO: 511 - RNA, 22 nt, Homo sapiens
aaaagcuggguugagagggcga

SEQ ID NO: 512 - RNA, 22 nt, Homo sapiens
cuggcccucucugcccuuccgu

SEQ ID NO: 513 - RNA, 22 nt, Homo sapiens
cuagguauggucccagggaucc

SEQ ID NO: 514 - RNA, 22 nt, Homo sapiens
aaaaugaaaugagcccagccca

SEQ ID NO: 515 - RNA, 22 nt, Homo sapiens
aauucccuuguagauaacccgg

SEQ ID NO: 516 - RNA, 22 nt, Homo sapiens
aucgggaaugucguguccgccc

SEQ ID NO: 517 - RNA, 22 nt, Homo sapiens
cagggcuggcagugacaugggu

SEQ ID NO: 518 - RNA, 22 nt, Homo sapiens
auuucccugccauucccuuggc

SEQ ID NO: 519 - RNA, 22 nt, Homo sapiens
gcucccucuagggucgcucgga

SEQ ID NO: 520 - RNA, 22 nt, Homo sapiens
aacccagugggcuauggaaaug

SEQ ID NO: 521 - RNA, 22 nt, Homo sapiens
ugggcuggcagggcaagugcug

SEQ ID NO: 522 - RNA, 22 nt, Homo sapiens
cagggccucacuguaucgccca

SEQ ID NO: 523 - RNA, 22 nt, Homo sapiens
aggacuggacucccggcagccc

SEQ ID NO: 524 - RNA, 22 nt, Homo sapiens
gcugaacugggcugagcugggc

SEQ ID NO: 525 - RNA, 22 nt, Homo sapiens
acugggaagaggagcugaggga

SEQ ID NO: 526 - RNA, 22 nt, Homo sapiens
ucucccuuccugcccuggcuag

SEQ ID NO: 527 - RNA, 22 nt, Homo sapiens
ugggauccagacagugggagaa

SEQ ID NO: 528 - RNA, 22 nt, Homo sapiens
uucucccacuaccaggcuccca

SEQ ID NO: 529 - RNA, 22 nt, Homo sapiens
acccagguucccucuggccgca

SEQ ID NO: 530 - RNA, 22 nt, Homo sapiens
aaucccaaugcuagacccggug

SEQ ID NO: 531 - RNA, 22 nt, Homo sapiens
gcugcgggcugcggucagggcg

SEQ ID NO: 532 - RNA, 22 nt, Homo sapiens
gcccgagaggauccgucccugc

SEQ ID NO: 533 - RNA, 22 nt, Homo sapiens
acccuugagccugaucccuagc

SEQ ID NO: 534 - RNA, 22 nt, Homo sapiens
aagacgggaggaaagaagggag

SEQ ID NO: 535 - RNA, 22 nt, Homo sapiens
aaugcacccgggcaaggauucu

SEQ ID NO: 536 - RNA, 22 nt, Homo sapiens
aauccuuugucccugggugaga

SEQ ID NO: 537 - RNA, 22 nt, Homo sapiens
ugggaugagggauugaagugga

SEQ ID NO: 538 - RNA, 22 nt, Homo sapiens
uaguucuucccuuugcccaauu

SEQ ID NO: 539 - RNA, 22 nt, Homo sapiens
cagggaaaugggaagaacuaga

SEQ ID NO: 540 - RNA, 22 nt, Homo sapiens
acagcccagcaguuaucacggg

SEQ ID NO: 541 - RNA, 22 nt, Homo sapiens
uccgagccugggucucccucuu

SEQ ID NO: 542 - RNA, 22 nt, Homo sapiens
guucucccaacguaagcccagc

SEQ ID NO: 543 - RNA, 22 nt, Homo sapiens
uccucuucucccuccucccagg

SEQ ID NO: 544 - RNA, 22 nt, Homo sapiens
gugaacgggcgccaucccgagg

SEQ ID NO: 545 - RNA, 22 nt, Homo sapiens
agggacgggacgcggugcagug

SEQ ID NO: 546 - RNA, 22 nt, Homo sapiens
ugugcgcagggagaccucuccc

SEQ ID NO: 547 - RNA, 22 nt, Homo sapiens
ugcccuuaaaggugaacccagu

SEQ ID NO: 548 - RNA, 22 nt, Homo sapiens
cccugugcccggcccacuucug

SEQ ID NO: 549 - RNA, 22 nt, Homo sapiens
ucacccugcaucccgcacccag

SEQ ID NO: 550 - RNA, 22 nt, Homo sapiens
cagcccggaucccagcccacuu

SEQ ID NO: 551 - RNA, 22 nt, Homo sapiens
cagcccuccucccgcacccaaa

SEQ ID NO: 552 - RNA, 22 nt, Homo sapiens
aaggcccgggcuuuccucccag

SEQ ID NO: 553 - RNA, 22 nt, Homo sapiens
cugcccuggcccgagggaccga

SEQ ID NO: 554 - RNA, 22 nt, Homo sapiens
gugggcugggcugggcugggcc

SEQ ID NO: 555 - RNA, 23 nt, Homo sapiens
agcagcauuguacagggcuauga

SEQ ID NO: 556 - RNA, 23 nt, Homo sapiens
agcagcauuguacagggcuauca

SEQ ID NO: 557 - RNA, 23 nt, Homo sapiens
uacccuguagauccgaauuugug

SEQ ID NO: 558 - RNA, 23 nt, Homo sapiens
uacccuguagaaccgaauuugug

SEQ ID NO: 559 - RNA, 23 nt, Homo sapiens
ucuggcuccgugucuucacuccc

SEQ ID NO: 560 - RNA, 23 nt, Homo sapiens
aacauucauugcugucggugggu

SEQ ID NO: 561 - RNA, 23 nt, Homo sapiens
aacauucauuguugucggugggu

SEQ ID NO: 562 - RNA, 23 nt, Homo sapiens
acugcccuaagugcuccuucugg

SEQ ID NO: 563 - RNA, 23 nt, Homo sapiens
caacggaaucccaaaagcagcug

SEQ ID NO: 564 - RNA, 23 nt, Homo sapiens
ugaguaccgccaugucuguuggg

SEQ ID NO: 565 - RNA, 23 nt, Homo sapiens
cccaguguucagacuaccuguuc

SEQ ID NO: 566 - RNA, 23 nt, Homo sapiens
cccaguguuuagacuaucuguuc

SEQ ID NO: 567 - RNA, 23 nt, Homo sapiens
uaauacugccggguaaugaugga

SEQ ID NO: 568 - RNA, 23 nt, Homo sapiens
agcuacauugucugcuggguuuc

SEQ ID NO: 569 - RNA, 23 nt, Homo sapiens
ucgaggacugguggaagggccuu

SEQ ID NO: 570 - RNA, 23 nt, Homo sapiens
cugacugaauagguagggucauu

SEQ ID NO: 571 - RNA, 23 nt, Homo sapiens
cugauaagaacagaggcccagau

SEQ ID NO: 572 - RNA, 23 nt, Homo sapiens
ccugggcagcguguggcugaagg

SEQ ID NO: 573 - RNA, 23 nt, Homo sapiens
ucugggagguuguagcaguggaa

SEQ ID NO: 574 - RNA, 23 nt, Homo sapiens
agggacugccuuaggagaaaguu

SEQ ID NO: 575 - RNA, 23 nt, Homo sapiens
ucccuguccuccaggagcucacg

SEQ ID NO: 576 - RNA, 23 nt, Homo sapiens
ucucacacagaaaucgcacccgu

SEQ ID NO: 577 - RNA, 23 nt, Homo sapiens
ugucugcccgcaugccugccucu

SEQ ID NO: 578 - RNA, 23 nt, Homo sapiens
ccacuuggaucugaaggcugccc

SEQ ID NO: 579 - RNA, 23 nt, Homo sapiens
cgagggcauuucaugaugcaggc

SEQ ID NO: 580 - RNA, 23 nt, Homo sapiens
caucagcacccuauguccuuucu

SEQ ID NO: 581 - RNA, 23 nt, Homo sapiens
ugagcaccacacaggccgggcgc

SEQ ID NO: 582 - RNA, 23 nt, Homo sapiens
accuggacccagcguagacaaag

SEQ ID NO: 583 - RNA, 23 nt, Homo sapiens
ucaaggccagaggucccacagca

SEQ ID NO: 584 - RNA, 23 nt, Homo sapiens
uucgggcuggccugcugcuccgg

SEQ ID NO: 585 - RNA, 23 nt, Homo sapiens
agguuacccgagcaacuuugcau

SEQ ID NO: 586 - RNA, 23 nt, Homo sapiens
aucaacagacauuaauugggcgc

SEQ ID NO: 587 - RNA, 23 nt, Homo sapiens
aaugacacgaucacucccguuga

SEQ ID NO: 588 - RNA, 23 nt, Homo sapiens
ucuuggaguaggucauugggugg

SEQ ID NO: 589 - RNA, 23 nt, Homo sapiens
gauugagacuaguagggcuaggc

SEQ ID NO: 590 - RNA, 23 nt, Homo sapiens
uagugcaauauugcuuauagggu

SEQ ID NO: 591 - RNA, 23 nt, Homo sapiens
uggaguuaaggguugcuuggaga

SEQ ID NO: 592 - RNA, 23 nt, Homo sapiens
uugcaugucagauuguaauuccc

SEQ ID NO: 593 - RNA, 23 nt, Homo sapiens
agggaaaaaaaaaaggauuuguc

SEQ ID NO: 594 - RNA, 23 nt, Homo sapiens
uggagauccagugcucgcccgau

SEQ ID NO: 595 - RNA, 23 nt, Homo sapiens
uaucugcugggcuuucugguguu

SEQ ID NO: 596 - RNA, 23 nt, Homo sapiens
ggcaggagggcugugccagguug

SEQ ID NO: 597 - RNA, 23 nt, Homo sapiens
agggccagaggagccuggagugg

SEQ ID NO: 598 - RNA, 23 nt, Homo sapiens
cuggcggagcccauuccaugcca

SEQ ID NO: 599 - RNA, 23 nt, Homo sapiens
uguagagcagggagcaggaagcu

SEQ ID NO: 600 - RNA, 23 nt, Homo sapiens
ucaggcaaagggauauuuacaga

SEQ ID NO: 601 - RNA, 23 nt, Homo sapiens
uggccggaugggacaggaggcau

SEQ ID NO: 602 - RNA, 23 nt, Homo sapiens
uggcccggcgacgucucacgguc

SEQ ID NO: 603 - RNA, 23 nt, Homo sapiens
ccggucccaggagaaccugcaga

SEQ ID NO: 604 - RNA, 23 nt, Homo sapiens
augcggaccuggguuagcggagu

SEQ ID NO: 605 - RNA, 23 nt, Homo sapiens
cagggaggcgcucacucucugcu

SEQ ID NO: 606 - RNA, 23 nt, Homo sapiens
cgcgggcgcuccuggccgccgcc

SEQ ID NO: 607 - RNA, 23 nt, Homo sapiens
aggaccugcgggacaagauucuu

SEQ ID NO: 608 - RNA, 23 nt, Homo sapiens
uaauccuugcuaccugggugaga

SEQ ID NO: 609 - RNA, 23 nt, Homo sapiens
ucacaacaaccuugcaggguaga

SEQ ID NO: 610 - RNA, 23 nt, Homo sapiens
uagcagcgggaacaguucugcag

SEQ ID NO: 611 - RNA, 23 nt, Homo sapiens
uacuccagagggcgucacucaug

SEQ ID NO: 612 - RNA, 23 nt, Homo sapiens
ggguuuguagcuuugcuggcaug

SEQ ID NO: 613 - RNA, 23 nt, Homo sapiens
augcuacucggaaaucccacuga

SEQ ID NO: 614 - RNA, 23 nt, Homo sapiens
aaucggacccauuuaaaccggag

SEQ ID NO: 615 - RNA, 23 nt, Homo sapiens
ugcacauggcaaccuagcuccca

SEQ ID NO: 616 - RNA, 23 nt, Homo sapiens
uaaaucccauggugccuucuccu

SEQ ID NO: 617 - RNA, 23 nt, Homo sapiens
acuugggcacugaaacaaugucc

SEQ ID NO: 618 - RNA, 23 nt, Homo sapiens
acugggaugucacugaauauggu

SEQ ID NO: 619 - RNA, 23 nt, Homo sapiens
uuugggauugacgccacaugucu

SEQ ID NO: 620 - RNA, 23 nt, Homo sapiens
ggcagguucucacccucucuagg

SEQ ID NO: 621 - RNA, 23 nt, Homo sapiens
ugucacucggcucggcccacuac

SEQ ID NO: 622 - RNA, 23 nt, Homo sapiens
aaggagcuuacaaucuagcuggg

SEQ ID NO: 623 - RNA, 23 nt, Homo sapiens
uccaguaccacgugucagggcca

SEQ ID NO: 624 - RNA, 23 nt, Homo sapiens
cgggucggaguuagcucaagcgg

SEQ ID NO: 625 - RNA, 23 nt, Homo sapiens
agguugggaucgguugcaaugcu

SEQ ID NO: 626 - RNA, 23 nt, Homo sapiens
cacccggcugugugcacaugugc

SEQ ID NO: 627 - RNA, 23 nt, Homo sapiens
gcuucuccuggcucuccucccuc

SEQ ID NO: 628 - RNA, 23 nt, Homo sapiens
cucucaccacugcccucccacag

SEQ ID NO: 629 - RNA, 23 nt, Homo sapiens
ccucagggcuguagaacagggcu

SEQ ID NO: 630 - RNA, 23 nt, Homo sapiens
guccaguuuucccaggaaucccu

SEQ ID NO: 631 - RNA, 23 nt, Homo sapiens
cggcccgggcugcugcuguuccu

SEQ ID NO: 632 - RNA, 23 nt, Homo sapiens
aucagggcuuguggaaugggaag

SEQ ID NO: 633 - RNA, 23 nt, Homo sapiens
uuuugcaugacccugggaguagg

SEQ ID NO: 634 - RNA, 23 nt, Homo sapiens
agggcauaggagaggguugauau

SEQ ID NO: 635 - RNA, 23 nt, Homo sapiens
ugacacggaggguggcuugggaa

SEQ ID NO: 636 - RNA, 23 nt, Homo sapiens
gagggcagcguggguguggcgga

SEQ ID NO: 637 - RNA, 23 nt, Homo sapiens
ugggcugagggcaggaggccugu

SEQ ID NO: 638 - RNA, 23 nt, Homo sapiens
cagggcucagggauuggauggag

SEQ ID NO: 639 - RNA, 23 nt, Homo sapiens
gacacgggcgacagcugcggccc

SEQ ID NO: 640 - RNA, 23 nt, Homo sapiens
ugugcuugcucgucccgcccgca

SEQ ID NO: 641 - RNA, 23 nt, Homo sapiens
uggugcggagagggcccacagug

SEQ ID NO: 642 - RNA, 23 nt, Homo sapiens
ugacaccugccacccagcccaag

SEQ ID NO: 643 - RNA, 23 nt, Homo sapiens
gagggucuugggagggaugugac

SEQ ID NO: 644 - RNA, 23 nt, Homo sapiens
ugggccagggagcagcugguggg

SEQ ID NO: 645 - RNA, 24 nt, Homo sapiens
uggcccugacugaagaccagcagu

SEQ ID NO: 646 - RNA, 24 nt, Homo sapiens
uugcagcugccugggagugacuuc

SEQ ID NO: 647 - RNA, 24 nt, Homo sapiens
agcgcgggcugagcgcugccaguc

SEQ ID NO: 648 - RNA, 24 nt, Homo sapiens
uggguagagaaggagcucagagga

SEQ ID NO: 649 - RNA, 24 nt, Homo sapiens
uguggacagugagguagagggagu

SEQ ID NO: 650 - RNA, 24 nt, Homo sapiens
cauagcccggucgcugguacauga

SEQ ID NO: 651 - RNA, 24 nt, Homo sapiens
cccggacaggcguucgugcgacgu

SEQ ID NO: 652 - RNA, 24 nt, Homo sapiens
guggaaagcaugcauccagggugu

SEQ ID NO: 653 - RNA, 24 nt, Homo sapiens
ugcccaugccauacuuuugccuca

SEQ ID NO: 654 - RNA, 24 nt, Homo sapiens
agaggacccguagcugcuagaagg

SEQ ID NO: 655 - RNA, 24 nt, Homo sapiens
ggugggauggagagaagguaugag

SEQ ID NO: 656 - RNA, 24 nt, Homo sapiens
acauccugcuccacagggcagagg

SEQ ID NO: 657 - RNA, 24 nt, Homo sapiens
agggcuggacucagcggcggagcu

SEQ ID NO: 658 - RNA, 24 nt, Homo sapiens
ucugggcacaggcggauggacagg

SEQ ID NO: 659 - RNA, 24 nt, Homo sapiens
ucagaacaaaugccgguucccaga

SEQ ID NO: 660 - RNA, 24 nt, Homo sapiens
gaccuggacauguuugugcccagu

SEQ ID NO: 661 - RNA, 24 nt, Homo sapiens
ugccugggucucuggccugcgcgu

SEQ ID NO: 662 - RNA, 24 nt, Homo sapiens
aaagugccgccuaguuuuaagccc

SEQ ID NO: 663 - RNA, 24 nt, Homo sapiens
ucccugagacccuuuaaccuguga

SEQ ID NO: 664 - RNA, 24 nt, Homo sapiens
agcagaagcagggagguucuccca

SEQ ID NO: 665 - RNA, 24 nt, Homo sapiens
gguugggcagugaggaggguguga

SEQ ID NO: 666 - RNA, 24 nt, Homo sapiens
acaaaaaaaaaagcccaacccuuc

SEQ ID NO: 667 - RNA, 24 nt, Homo sapiens
ucccugaggagcccuuugagccug

SEQ ID NO: 668 - RNA, 25 nt, Homo sapiens
gggcgacaaagcaagacucuuucuu

SEQ ID NO: 669 - RNA, 25 nt, Homo sapiens
ggcggagggaaguagguccguuggu

SEQ ID NO: 670 - RNA, 25 nt, Homo sapiens
cuagugagggacagaaccaggauuc

SEQ ID NO: 671 - RNA, 25 nt, Homo sapiens
ugggaacggguuccggcagacgcug

SEQ ID NO: 672 - RNA, 25 nt, Homo sapiens
gcugggcagggcuucugagcuccuu

SEQ ID NO: 673 - RNA, 25 nt, Homo sapiens
agggaucgcgggcggguggcggccu

SEQ ID NO: 674 - RNA, 26 nt, Homo sapiens
gcucagggaugauaacugugcugaga

SEQ ID NO: 675 - RNA, 27 nt, Homo sapiens
cacuguaggugauggugagagugggca

SEQ ID NO: 676 - RNA, 17 nt, Homo sapiens
ggggcgcggccggaucg

SEQ ID NO: 677 - RNA, 17 nt, Homo sapiens
ccccgccaccgccuugg

SEQ ID NO: 678 - RNA, 17 nt, Homo sapiens
uggggcucagcgaguuu

SEQ ID NO: 679 - RNA, 17 nt, Homo sapiens
accccacuccugguacc

SEQ ID NO: 680 - RNA, 17 nt, Homo sapiens
gggguggucuguuguug

SEQ ID NO: 681 - RNA, 17 nt, Homo sapiens
ggauggaggaggggucu

SEQ ID NO: 682 - RNA, 17 nt, Homo sapiens
gagacugggguggggcc

SEQ ID NO: 683 - RNA, 17 nt, Homo sapiens
ggggcugggcgcgcgcc

SEQ ID NO: 684 - RNA, 17 nt, Homo sapiens
gcggggcugggcgcgcg

SEQ ID NO: 685 - RNA, 17 nt, Homo sapiens
gggagaagggucggggc

SEQ ID NO: 686 - RNA, 17 nt, Homo sapiens
ccccggggagcccggcg

SEQ ID NO: 687 - RNA, 18 nt, Homo sapiens
agcaggugcggggcggcg

SEQ ID NO: 688 - RNA, 18 nt, Homo sapiens
ccagagguggggacugag

SEQ ID NO: 689 - RNA, 18 nt, Homo sapiens
cagccccacagccucaga

SEQ ID NO: 690 - RNA, 18 nt, Homo sapiens
acaggcaggauuggggaa

SEQ ID NO: 691 - RNA, 18 nt, Homo sapiens
aucucgcuggggccucca

SEQ ID NO: 692 - RNA, 18 nt, Homo sapiens
cggggcggcaggggccuc

SEQ ID NO: 693 - RNA, 18 nt, Homo sapiens
gggcucacaucaccccau

SEQ ID NO: 694 - RNA, 18 nt, Homo sapiens
ccccugggccggccuugg

SEQ ID NO: 695 - RNA, 18 nt, Homo sapiens
gggugcgggccggcgggg

SEQ ID NO: 696 - RNA, 19 nt, Homo sapiens
uggggcggagcuuccggag

SEQ ID NO: 697 - RNA, 19 nt, Homo sapiens
ggccuuguuccugucccca

SEQ ID NO: 698 - RNA, 19 nt, Homo sapiens
ugucucugcugggguuucu

SEQ ID NO: 699 - RNA, 19 nt, Homo sapiens
cagcaggaggugaggggag

SEQ ID NO: 700 - RNA, 19 nt, Homo sapiens
acuugaggggcaugaggau

SEQ ID NO: 701 - RNA, 19 nt, Homo sapiens
ggggccuggcggugggcgg

SEQ ID NO: 702 - RNA, 19 nt, Homo sapiens
cuuggggcauggaguccca

SEQ ID NO: 703 - RNA, 19 nt, Homo sapiens
cggcgcgaccggcccgggg

SEQ ID NO: 704 - RNA, 19 nt, Homo sapiens
agcagggcuggggauugca

SEQ ID NO: 705 - RNA, 19 nt, Homo sapiens
ggggagcgaggggcggggc

SEQ ID NO: 706 - RNA, 20 nt, Homo sapiens
cuuccucgucugucugcccc

SEQ ID NO: 707 - RNA, 20 nt, Homo sapiens
uuggccauggggcugcgcgg

SEQ ID NO: 708 - RNA, 20 nt, Homo sapiens
ucuggccagcuacgucccca

SEQ ID NO: 709 - RNA, 20 nt, Homo sapiens
acugccccaggugcugcugg

SEQ ID NO: 710 - RNA, 20 nt, Homo sapiens
gguauccguuuggggauggu

SEQ ID NO: 711 - RNA, 20 nt, Homo sapiens
ggcuccuuggucuaggggua

SEQ ID NO: 712 - RNA, 20 nt, Homo sapiens
gcggagagagaauggggagc

SEQ ID NO: 713 - RNA, 20 nt, Homo sapiens
accaggaggcugaggccccu

SEQ ID NO: 714 - RNA, 20 nt, Homo sapiens
ggggagcuguggaagcagua

SEQ ID NO: 715 - RNA, 20 nt, Homo sapiens
cgugccacccuuuuccccag

SEQ ID NO: 716 - RNA, 20 nt, Homo sapiens
cgcgggucggggucugcagg

SEQ ID NO: 717 - RNA, 20 nt, Homo sapiens
aaaaggcgggagaagcccca

SEQ ID NO: 718 - RNA, 20 nt, Homo sapiens
cggcucugggucugugggga

SEQ ID NO: 719 - RNA, 20 nt, Homo sapiens
ccccagggcgacgcggcggg

SEQ ID NO: 720 - RNA, 20 nt, Homo sapiens
guggguuggggcgggcucug

SEQ ID NO: 721 - RNA, 20 nt, Homo sapiens
ucagggagucaggggagggc

SEQ ID NO: 722 - RNA, 20 nt, Homo sapiens
cggggugggugaggucgggc

SEQ ID NO: 723 - RNA, 20 nt, Homo sapiens
gggcuggggcgcggggaggu

SEQ ID NO: 724 - RNA, 20 nt, Homo sapiens
ggugggagguggggugggca

SEQ ID NO: 725 - RNA, 21 nt, Homo sapiens
ccccaccuccucucuccucag

SEQ ID NO: 726 - RNA, 21 nt, Homo sapiens
ccuguugaaguguaaucccca

SEQ ID NO: 727 - RNA, 21 nt, Homo sapiens
cggcggggacggcgauugguc

SEQ ID NO: 728 - RNA, 21 nt, Homo sapiens
auccccagauacaauggacaa

SEQ ID NO: 729 - RNA, 21 nt, Homo sapiens
ugcacggcacuggggacacgu

SEQ ID NO: 730 - RNA, 21 nt, Homo sapiens
aggggugcuaucugugauuga

SEQ ID NO: 731 - RNA, 21 nt, Homo sapiens
uggggcuagugaugcaggacg

SEQ ID NO: 732 - RNA, 21 nt, Homo sapiens
ucugaggccugccucucccca

SEQ ID NO: 733 - RNA, 21 nt, Homo sapiens
gagguuuggggaggauuugcu

SEQ ID NO: 734 - RNA, 21 nt, Homo sapiens
uagcggggauuccaauauugg

SEQ ID NO: 735 - RNA, 21 nt, Homo sapiens
cggggcagcucaguacaggau

SEQ ID NO: 736 - RNA, 21 nt, Homo sapiens
uacuuuucuagguuguugggg

SEQ ID NO: 737 - RNA, 21 nt, Homo sapiens
gguggggcaaugggaucaggu

SEQ ID NO: 738 - RNA, 21 nt, Homo sapiens
gccccugggccuauccuagaa

SEQ ID NO: 739 - RNA, 21 nt, Homo sapiens
agccgcggggaucgccgaggg

SEQ ID NO: 740 - RNA, 21 nt, Homo sapiens
cugugggcucagcgcgugggg

SEQ ID NO: 741 - RNA, 21 nt, Homo sapiens
ucccuccuucuguccccacag

SEQ ID NO: 742 - RNA, 21 nt, Homo sapiens
guuggggugcaggggucugcu

SEQ ID NO: 743 - RNA, 21 nt, Homo sapiens
gccccgggcagugugaucauc

SEQ ID NO: 744 - RNA, 21 nt, Homo sapiens
cguccccgcucggcggggucc

SEQ ID NO: 745 - RNA, 21 nt, Homo sapiens
uggcagggaggcugggagggg

SEQ ID NO: 746 - RNA, 21 nt, Homo sapiens
gcccuccgcccgugcaccccg

SEQ ID NO: 747 - RNA, 21 nt, Homo sapiens
ucccuaccccuccacucccca

SEQ ID NO: 748 - RNA, 21 nt, Homo sapiens
cccuugggucugaugggguag

SEQ ID NO: 749 - RNA, 21 nt, Homo sapiens
acccucgucagguccccgggg

SEQ ID NO: 750 - RNA, 21 nt, Homo sapiens
ugcccacccuuuaccccgcuc

SEQ ID NO: 751 - RNA, 22 nt, Homo sapiens
caaauucguaucuaggggaaua

SEQ ID NO: 752 - RNA, 22 nt, Homo sapiens
acagauucgauucuaggggaau

SEQ ID NO: 753 - RNA, 22 nt, Homo sapiens
ccucuuccccuugucucuccag

SEQ ID NO: 754 - RNA, 22 nt, Homo sapiens
uuuagagacggggucuugcucu

SEQ ID NO: 755 - RNA, 22 nt, Homo sapiens
ucaaaacugaggggcauuuucu

SEQ ID NO: 756 - RNA, 22 nt, Homo sapiens
uuugguccccuucaaccagcug

SEQ ID NO: 757 - RNA, 22 nt, Homo sapiens
uuugguccccuucaaccagcua

SEQ ID NO: 758 - RNA, 22 nt, Homo sapiens
ugugacugguugaccagagggg

SEQ ID NO: 759 - RNA, 22 nt, Homo sapiens
aggggcuggcuuuccucugguc

SEQ ID NO: 760 - RNA, 22 nt, Homo sapiens
gcugcgcuuggauuucgucccc

SEQ ID NO: 761 - RNA, 22 nt, Homo sapiens
ccaguggggcugcuguuaucug

SEQ ID NO: 762 - RNA, 22 nt, Homo sapiens
gguccagaggggagauagguuc

SEQ ID NO: 763 - RNA, 22 nt, Homo sapiens
uuggggaaacggccgcugagug

SEQ ID NO: 764 - RNA, 22 nt, Homo sapiens
ugucaguuugucaaauacccca

SEQ ID NO: 765 - RNA, 22 nt, Homo sapiens
uguaaacauccccgacuggaag

SEQ ID NO: 766 - RNA, 22 nt, Homo sapiens
uccccuucugcaggccugcugg

SEQ ID NO: 767 - RNA, 22 nt, Homo sapiens
aggggaccaaagagauauauag

SEQ ID NO: 768 - RNA, 22 nt, Homo sapiens
cuggggagauccucgagguugg

SEQ ID NO: 769 - RNA, 22 nt, Homo sapiens
caaccucgaggaucuccccagc

SEQ ID NO: 770 - RNA, 22 nt, Homo sapiens
uguguuagaauaggggcaauaa

SEQ ID NO: 771 - RNA, 22 nt, Homo sapiens
cugggguucugagacagacagu

SEQ ID NO: 772 - RNA, 22 nt, Homo sapiens
cggggagagaacgcagugacgu

SEQ ID NO: 773 - RNA, 22 nt, Homo sapiens
agaaggggugaaauuuaaacgu

SEQ ID NO: 774 - RNA, 22 nt, Homo sapiens
uggggcggagcuuccggaggcc

SEQ ID NO: 775 - RNA, 22 nt, Homo sapiens
guggaguccuggggaauggaga

SEQ ID NO: 776 - RNA, 22 nt, Homo sapiens
uuaucagaaucuccagggguac

SEQ ID NO: 777 - RNA, 22 nt, Homo sapiens
uaaugccccuaaaaauccuuau

SEQ ID NO: 778 - RNA, 22 nt, Homo sapiens
gccugcugggguggaaccuggu

SEQ ID NO: 779 - RNA, 22 nt, Homo sapiens
gaaggcagcagugcuccccugu

SEQ ID NO: 780 - RNA, 22 nt, Homo sapiens
uaagggguguauggcagaugca

SEQ ID NO: 781 - RNA, 22 nt, Homo sapiens
gaauguugcucggugaaccccu

SEQ ID NO: 782 - RNA, 22 nt, Homo sapiens
ugucguggggcuugcuggcuug

SEQ ID NO: 783 - RNA, 22 nt, Homo sapiens
uggggauuuggagaagugguga

SEQ ID NO: 784 - RNA, 22 nt, Homo sapiens
cucaaguagucugaccagggga

SEQ ID NO: 785 - RNA, 22 nt, Homo sapiens
uuucuauuucucaguggggcuc

SEQ ID NO: 786 - RNA, 22 nt, Homo sapiens
auggcaucguccccugguggcu

SEQ ID NO: 787 - RNA, 22 nt, Homo sapiens
aggggacugguuaauagaacua

SEQ ID NO: 788 - RNA, 22 nt, Homo sapiens
cuuccggucugugagccccguc

SEQ ID NO: 789 - RNA, 22 nt, Homo sapiens
acuggggagcagaaggagaacc

SEQ ID NO: 790 - RNA, 22 nt, Homo sapiens
ggggcugugauugaccagcagg

SEQ ID NO: 791 - RNA, 22 nt, Homo sapiens
ucuggggaugaggacagugugu

SEQ ID NO: 792 - RNA, 22 nt, Homo sapiens
uuggccaccacaccuaccccuu

SEQ ID NO: 793 - RNA, 22 nt, Homo sapiens
aacucugaccccuuagguugau

SEQ ID NO: 794 - RNA, 22 nt, Homo sapiens
uccuguacugagcugccccgag

SEQ ID NO: 795 - RNA, 22 nt, Homo sapiens
aaccccuaaggcaacuggaugg

SEQ ID NO: 796 - RNA, 22 nt, Homo sapiens
aaccagcaccccaacuuuggac

SEQ ID NO: 797 - RNA, 22 nt, Homo sapiens
uuggaauaggggauaucucagc

SEQ ID NO: 798 - RNA, 22 nt, Homo sapiens
cagcaggggagagagaggaguc

SEQ ID NO: 799 - RNA, 22 nt, Homo sapiens
aggcgauguggggauguagaga

SEQ ID NO: 800 - RNA, 22 nt, Homo sapiens
auaguccgaguaacgucggggc

SEQ ID NO: 801 - RNA, 22 nt, Homo sapiens
acuccagccccacagccucagc

SEQ ID NO: 802 - RNA, 22 nt, Homo sapiens
aggcagcgggguguaguggaua

SEQ ID NO: 803 - RNA, 22 nt, Homo sapiens
ccaagucuuggggagaguugag

SEQ ID NO: 804 - RNA, 22 nt, Homo sapiens
uauucauuuacuccccagccua

SEQ ID NO: 805 - RNA, 22 nt, Homo sapiens
ucggccugaccacccaccccac

SEQ ID NO: 806 - RNA, 22 nt, Homo sapiens
aagcccuuaccccaaaaaguau

SEQ ID NO: 807 - RNA, 22 nt, Homo sapiens
aagcccuuaccccaaaaagcau

SEQ ID NO: 808 - RNA, 22 nt, Homo sapiens
cucggcgcggggcgcgggcucc

SEQ ID NO: 809 - RNA, 22 nt, Homo sapiens
ugcccuaaaugccccuucuggc

SEQ ID NO: 810 - RNA, 22 nt, Homo sapiens
cgcaggggccgggugcucaccg

SEQ ID NO: 811 - RNA, 22 nt, Homo sapiens
cgggguuuugagggcgagauga

SEQ ID NO: 812 - RNA, 22 nt, Homo sapiens
cagaaggggaguugggagcaga

SEQ ID NO: 813 - RNA, 22 nt, Homo sapiens
cugccagccccguuccagggca

SEQ ID NO: 814 - RNA, 22 nt, Homo sapiens
caguugggucuaggggucagga

SEQ ID NO: 815 - RNA, 22 nt, Homo sapiens
cgucccggggcugcgcgaggca

SEQ ID NO: 816 - RNA, 22 nt, Homo sapiens
guucuguuaacccauccccuca

SEQ ID NO: 817 - RNA, 22 nt, Homo sapiens
caccggggauggcagagggucg

SEQ ID NO: 818 - RNA, 22 nt, Homo sapiens
uggggugcccacuccgcaaguu

SEQ ID NO: 819 - RNA, 22 nt, Homo sapiens
uaggggcagcagaggaccuggg

SEQ ID NO: 820 - RNA, 22 nt, Homo sapiens
agcccgccccagccgagguucu

SEQ ID NO: 821 - RNA, 22 nt, Homo sapiens
cccucucuggcuccuccccaaa

SEQ ID NO: 822 - RNA, 22 nt, Homo sapiens
uggggaaggcgucagugucggg

SEQ ID NO: 823 - RNA, 22 nt, Homo sapiens
ugcggggacaggccagggcauc

SEQ ID NO: 824 - RNA, 22 nt, Homo sapiens
ucugccauccucccuccccuac

SEQ ID NO: 825 - RNA, 22 nt, Homo sapiens
ucaggcucaguccccucccgau

SEQ ID NO: 826 - RNA, 22 nt, Homo sapiens
aguggggaacccuuccaugagg

SEQ ID NO: 827 - RNA, 22 nt, Homo sapiens
ugaggcccuuggggcacagugg

SEQ ID NO: 828 - RNA, 22 nt, Homo sapiens
uuuugugucucccauuccccag

SEQ ID NO: 829 - RNA, 22 nt, Homo sapiens
ugagggguuuggaaugggaugg

SEQ ID NO: 830 - RNA, 22 nt, Homo sapiens
aggcggggcgccgcgggaccgc

SEQ ID NO: 831 - RNA, 22 nt, Homo sapiens
ugcggggcuagggcuaacagca

SEQ ID NO: 832 - RNA, 22 nt, Homo sapiens
ggguggggauuuguugcauuac

SEQ ID NO: 833 - RNA, 22 nt, Homo sapiens
acccgucccguucguccccgga

SEQ ID NO: 834 - RNA, 22 nt, Homo sapiens
ggaggggucccgcacugggagg

SEQ ID NO: 835 - RNA, 22 nt, Homo sapiens
gggguuccuggggaugggauuu

SEQ ID NO: 836 - RNA, 22 nt, Homo sapiens
cuugguucagggagggucccca

SEQ ID NO: 837 - RNA, 22 nt, Homo sapiens
ugcaggggucgggugggccagg

SEQ ID NO: 838 - RNA, 22 nt, Homo sapiens
gggacccggggagagauguaag

SEQ ID NO: 839 - RNA, 22 nt, Homo sapiens
ggggcuggggccgggacagagc

SEQ ID NO: 840 - RNA, 22 nt, Homo sapiens
agggaaggggacgaggguuggg

SEQ ID NO: 841 - RNA, 23 nt, Homo sapiens
uuaaugcuaaucgugauaggggu

SEQ ID NO: 842 - RNA, 23 nt, Homo sapiens
aaagucucgcucucugccccuca

SEQ ID NO: 843 - RNA, 23 nt, Homo sapiens
agaggcuuugugcggauacgggg

SEQ ID NO: 844 - RNA, 23 nt, Homo sapiens
uggggacguagcuggccagacag

SEQ ID NO: 845 - RNA, 23 nt, Homo sapiens
uauggggcuucuguagagauuuc

SEQ ID NO: 846 - RNA, 23 nt, Homo sapiens
gaagugcuucgauuuuggggugu

SEQ ID NO: 847 - RNA, 23 nt, Homo sapiens
agcucggucugaggccccucagu

SEQ ID NO: 848 - RNA, 23 nt, Homo sapiens
ugaggggcagagagcgagacuuu

SEQ ID NO: 849 - RNA, 23 nt, Homo sapiens
ucaaaauguagaggaagacccca

SEQ ID NO: 850 - RNA, 23 nt, Homo sapiens
uguuucggggcucauggccugug

SEQ ID NO: 851 - RNA, 23 nt, Homo sapiens
aggucugcauucaaauccccaga

SEQ ID NO: 852 - RNA, 23 nt, Homo sapiens
caggcagaaguggggcugacagg

SEQ ID NO: 853 - RNA, 23 nt, Homo sapiens
ccucaccaccccuucugccugca

SEQ ID NO: 854 - RNA, 23 nt, Homo sapiens
ucugcucauaccccaugguuucu

SEQ ID NO: 855 - RNA, 23 nt, Homo sapiens
uccggggcugaguucugugcacc

SEQ ID NO: 856 - RNA, 23 nt, Homo sapiens
ggggaaagcgaguagggacauuu

SEQ ID NO: 857 - RNA, 23 nt, Homo sapiens
cgcauccccuagggcauuggugu

SEQ ID NO: 858 - RNA, 23 nt, Homo sapiens
ugaggauauggcagggaagggga

SEQ ID NO: 859 - RNA, 23 nt, Homo sapiens
ugggaggggagaggcagcaagca

SEQ ID NO: 860 - RNA, 23 nt, Homo sapiens
cgggcuguccggaggggucggcu

SEQ ID NO: 861 - RNA, 23 nt, Homo sapiens
ugccccaccugcugaccacccuc

SEQ ID NO: 862 - RNA, 23 nt, Homo sapiens
gugagugggagccgguggggcug

SEQ ID NO: 863 - RNA, 23 nt, Homo sapiens
ccccgguguuggggcgcgucugc

SEQ ID NO: 864 - RNA, 23 nt, Homo sapiens
ccggggcagauugguguagggug

SEQ ID NO: 865 - RNA, 23 nt, Homo sapiens
gcgaggaccccucggggucugac

SEQ ID NO: 866 - RNA, 23 nt, Homo sapiens
aggaagcccuggaggggcuggag

SEQ ID NO: 867 - RNA, 23 nt, Homo sapiens
ugggcaggggcuuauuguaggag

SEQ ID NO: 868 - RNA, 23 nt, Homo sapiens
cugggaucuccggggucuugguu

SEQ ID NO: 869 - RNA, 23 nt, Homo sapiens
uuagggaguagaaggguggggag

SEQ ID NO: 870 - RNA, 23 nt, Homo sapiens
gccccggcgcgggcggguucugg

SEQ ID NO: 871 - RNA, 23 nt, Homo sapiens
ugaguggggcucccgggacggcg

SEQ ID NO: 872 - RNA, 23 nt, Homo sapiens
aucccuugcaggggcuguugggu

SEQ ID NO: 873 - RNA, 23 nt, Homo sapiens
cugggcccgcggcgggcgugggg

SEQ ID NO: 874 - RNA, 24 nt, Homo sapiens
ugaggggccucagaccgagcuuuu

SEQ ID NO: 875 - RNA, 24 nt, Homo sapiens
ugucagugacuccugccccuuggu

SEQ ID NO: 876 - RNA, 24 nt, Homo sapiens
uuggacuuuuucagauuuggggau

SEQ ID NO: 877 - RNA, 24 nt, Homo sapiens
cugcaggcagaaguggggcugaca

SEQ ID NO: 878 - RNA, 24 nt, Homo sapiens
ucuguagccugggagcaauggggu

SEQ ID NO: 879 - RNA, 24 nt, Homo sapiens
gaugcgccgcccacugccccgcgc

SEQ ID NO: 880 - RNA, 24 nt, Homo sapiens
ugggcgaggggugggcucucagag

SEQ ID NO: 881 - RNA, 24 nt, Homo sapiens
aggcaggggcuggugcugggcggg

SEQ ID NO: 882 - RNA, 24 nt, Homo sapiens
ggaggccgggguggggcggggcgg

SEQ ID NO: 883 - RNA, 25 nt, Homo sapiens
cuuccagacgcuccgccccacgucg

SEQ ID NO: 884 - RNA, 25 nt, Homo sapiens
agcggggaggaagugggcgcugcuu

SEQ ID NO: 885 - RNA, 25 nt, Homo sapiens
caugcugaccucccuccugccccag

SEQ ID NO: 886 - RNA, 25 nt, Homo sapiens
aggggugguguugggacagcuccgu

SEQ ID NO: 887 - RNA, 25 nt, Homo sapiens
ugccccaucugugcccuggguagga

SEQ ID NO: 888 - RNA, 25 nt, Homo sapiens
aagggaggaggagcggaggggcccu

SEQ ID NO: 889 - RNA, 26 nt, Homo sapiens
cacaggacugacuccucaccccagug

SEQ ID NO: 890 - RNA, 26 nt, Homo sapiens
gugagggcaugcaggccuggaugggg

SEQ ID NO: 891 - RNA, 26 nt, Homo sapiens
cccagggcuuggaguggggcaagguu

SEQ ID NO: 892 - RNA, 17 nt, Homo sapiens
gugggggagaggcuguc

SEQ ID NO: 893 - RNA, 17 nt, Homo sapiens
ggugggggcuguuguuu

SEQ ID NO: 894 - RNA, 17 nt, Homo sapiens
ggcgggugcgggggugg

SEQ ID NO: 895 - RNA, 18 nt, Homo sapiens
cgggcguggugguggggg

SEQ ID NO: 896 - RNA, 18 nt, Homo sapiens
agggcauguccagggggu

SEQ ID NO: 897 - RNA, 18 nt, Homo sapiens
cagcagucccucccccug

SEQ ID NO: 898 - RNA, 18 nt, Homo sapiens
cuaggggguuugcccuug

SEQ ID NO: 899 - RNA, 18 nt, Homo sapiens
agggggcgggcuccggcg

SEQ ID NO: 900 - RNA, 19 nt, Homo sapiens
ggcuugcaugggggacugg

SEQ ID NO: 901 - RNA, 19 nt, Homo sapiens
gggggaagaaaaggugggg

SEQ ID NO: 902 - RNA, 19 nt, Homo sapiens
aaggggcugggggagcaca

SEQ ID NO: 903 - RNA, 20 nt, Homo sapiens
uaggaugggggugagaggug

SEQ ID NO: 904 - RNA, 20 nt, Homo sapiens
ccguguuucccccacgcuuu

SEQ ID NO: 905 - RNA, 20 nt, Homo sapiens
acucaaacugugggggcacu

SEQ ID NO: 906 - RNA, 20 nt, Homo sapiens
ggcggcggcggaggcggggg

SEQ ID NO: 907 - RNA, 20 nt, Homo sapiens
cgccccuccugcccccacag

SEQ ID NO: 908 - RNA, 20 nt, Homo sapiens
gggaaaaggaagggggagga

SEQ ID NO: 909 - RNA, 20 nt, Homo sapiens
agcccccuggccccaaaccc

SEQ ID NO: 910 - RNA, 20 nt, Homo sapiens
ugggaauggggguaagggcc

SEQ ID NO: 911 - RNA, 21 nt, Homo sapiens
gugccagcugcagugggggag

SEQ ID NO: 912 - RNA, 21 nt, Homo sapiens
uccuucugcuccgucccccag

SEQ ID NO: 913 - RNA, 21 nt, Homo sapiens
uggcuguuggagggggcaggc

SEQ ID NO: 914 - RNA, 21 nt, Homo sapiens
auccaguucucugagggggcu

SEQ ID NO: 915 - RNA, 21 nt, Homo sapiens
agggggaaaguucuauagucc

SEQ ID NO: 916 - RNA, 21 nt, Homo sapiens
cuccgugcacacccccgcgug

SEQ ID NO: 917 - RNA, 21 nt, Homo sapiens
cagugcaauuaaaagggggaa

SEQ ID NO: 918 - RNA, 21 nt, Homo sapiens
gugggcgggggcaggugugug

SEQ ID NO: 919 - RNA, 21 nt, Homo sapiens
acaggagugggggugggacau

SEQ ID NO: 920 - RNA, 21 nt, Homo sapiens
gggcaucugcugacauggggg

SEQ ID NO: 921 - RNA, 21 nt, Homo sapiens
agggagggacgggggcugugc

SEQ ID NO: 922 - RNA, 21 nt, Homo sapiens
gggcuagggccugcugccccc

SEQ ID NO: 923 - RNA, 21 nt, Homo sapiens
aaggcagggcccccgcucccc

SEQ ID NO: 924 - RNA, 22 nt, Homo sapiens
cugguacaggccugggggacag

SEQ ID NO: 925 - RNA, 22 nt, Homo sapiens
ucugcccccuccgcugcugcca

SEQ ID NO: 926 - RNA, 22 nt, Homo sapiens
uuuaacauggggguaccugcug

SEQ ID NO: 927 - RNA, 22 nt, Homo sapiens
caucucuaaggaacucccccaa

SEQ ID NO: 928 - RNA, 22 nt, Homo sapiens
acucaaaaugggggcgcuuucc

SEQ ID NO: 929 - RNA, 22 nt, Homo sapiens
uugaggagacauggugggggcc

SEQ ID NO: 930 - RNA, 22 nt, Homo sapiens
agggggcgcagucacugacgug

SEQ ID NO: 931 - RNA, 22 nt, Homo sapiens
aagggggaaggaaacauggaga

SEQ ID NO: 932 - RNA, 22 nt, Homo sapiens
uccauguuuccuucccccuucu

SEQ ID NO: 933 - RNA, 22 nt, Homo sapiens
cacacaaguggcccccaacacu

SEQ ID NO: 934 - RNA, 22 nt, Homo sapiens
ugcugggggccacaugagugug

SEQ ID NO: 935 - RNA, 22 nt, Homo sapiens
agggggauggcagagcaaaauu

SEQ ID NO: 936 - RNA, 22 nt, Homo sapiens
ugggggagugcagugauugugg

SEQ ID NO: 937 - RNA, 22 nt, Homo sapiens
gacuauagaacuuucccccuca

SEQ ID NO: 938 - RNA, 22 nt, Homo sapiens
  misc_feature: “n” is abasic or substituted base
acucaaacuaugggggcacuuu

SEQ ID NO: 939 - RNA, 22 nt, Homo sapiens
acucaaacugggggcucuuuug

SEQ ID NO: 940 - RNA, 22 nt, Homo sapiens
ugagccccugugccgcccccag

SEQ ID NO: 941 - RNA, 22 nt, Homo sapiens
cacccccuguuuccuggcccac

SEQ ID NO: 942 - RNA, 22 nt, Homo sapiens
gcggggguggcggcggcauccc

SEQ ID NO: 943 - RNA, 22 nt, Homo sapiens
ggggguccccggugcucggauc

SEQ ID NO: 944 - RNA, 22 nt, Homo sapiens
ccugacccacccccucccgcag

SEQ ID NO: 945 - RNA, 23 nt, Homo sapiens
ucccccaggugugauucugauuu

SEQ ID NO: 946 - RNA, 23 nt, Homo sapiens
acaggcggcuguagcaauggggg

SEQ ID NO: 947 - RNA, 23 nt, Homo sapiens
uagcccccaggcuucacuuggcg

SEQ ID NO: 948 - RNA, 23 nt, Homo sapiens
cugggggacgcgugagcgcgagc

SEQ ID NO: 949 - RNA, 23 nt, Homo sapiens
uuucaagccagggggcguuuuuc

SEQ ID NO: 950 - RNA, 24 nt, Homo sapiens
ugggggagccaugagauaagagca

SEQ ID NO: 951 - RNA, 24 nt, Homo sapiens
acugggggcuuucgggcucugcgu

SEQ ID NO: 952 - RNA, 24 nt, Homo sapiens
uggggagcugaggcucugggggug

SEQ ID NO: 953 - RNA, 25 nt, Homo sapiens
cccccacaaccgcgcuugacuagcu

SEQ ID NO: 954 - RNA, 26 nt, Homo sapiens
cucggccgcggcgcguagcccccgcc

SEQ ID NO: 955 - RNA, 18 nt, Homo sapiens
ggugggggguguuguuuu

SEQ ID NO: 956 - RNA, 18 nt, Homo sapiens
gggucccggggagggggg

SEQ ID NO: 957 - RNA, 20 nt, Homo sapiens
ucacaccugccucgcccccc

SEQ ID NO: 958 - RNA, 20 nt, Homo sapiens
ucucuucaucuaccccccag

SEQ ID NO: 959 - RNA, 21 nt, Homo sapiens
ggggggaugugcaugcugguu

SEQ ID NO: 960 - RNA, 21 nt, Homo sapiens
cgucccaccccccacuccugu

SEQ ID NO: 961 - RNA, 22 nt, Homo sapiens
cuuccccccaguaaucuucauc

SEQ ID NO: 962 - RNA, 22 nt, Homo sapiens
guggguacggcccagugggggg

SEQ ID NO: 963 - RNA, 18 nt, Homo sapiens
ugaggcgggggggcgagc

SEQ ID NO: 964 - RNA, 20 nt, Homo sapiens
agagaugaagcgggggggcg

SEQ ID NO: 965 - RNA, 21 nt, Homo sapiens
agggcccccccucaauccugu

SEQ ID NO: 966 - RNA, 22 nt, Homo sapiens
acgcccuucccccccuucuuca

SEQ ID NO: 967 - DNA, 22 bp, Artificial Sequence
  probe
tcgccctctcaacccagctttt

SEQ ID NO: 968 - DNA, 22 bp, Artificial Sequence
  probe
  misc_feature (14)..(14): “n” is abasic
tcgccctctcaacncagctttt

SEQ ID NO: 969 - DNA, 22 bp, Artificial Sequence
  probe
  misc_feature (5)..(5): “n” is abasic
tcgcnctctcaacccagctttt

SEQ ID NO: 970 - DNA, 22 bp, Artificial Sequence
  probe
  misc_feature (5)..(5): “n” is abasic
  misc_feature (14)..(14): “n” is abasic
tcgcnctctcaacncagctttt

SEQ ID NO: 971 - RNA, 22 nt, Artificial Sequence
  target sequence
aaaagcuggguugagagggcga

SEQ ID NO: 972 - RNA, 21 nt, Artificial Sequence
  non-target sequence
uggcagggaggcugggagggg

SEQ ID NO: 973 - RNA, 23 nt, Artificial Sequence
  target sequence
agcuacauugucugcuggguuuc

SEQ ID NO: 974 - RNA, 21 nt, Artificial Sequence
  non-target sequence
uggcagggaggcugggagggg

SEQ ID NO: 975 - DNA, 23 bp, Artificial Sequence
  probe
gaaacccagcagacaatgtagct

SEQ ID NO: 976 - DNA, 18 bp, Artificial Sequence
  probe
ccagcagacaatgtagct

SEQ ID NO: 977 - DNA, 17 bp, Artificial Sequence
  probe
cagcagacaatgtagct